(19) United States
(12) Patent Application Publication
(10) Pub. No.: US 2006/0003924 A1
(43) Pub. Date: Jan. 5, 2006

(54) Sulfation-independent L-selectin or E-selectin ligand (HCELL) and therapeutics thereof

(76) Inventor: Robert Sackstein, Sudbury, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/032,256

(22) Filed: Jan. 10, 2005

Related U.S. Application Data
(63) Continuation of application No. 10/949,799, filed on Sep. 24, 2004, now abandoned.
Application No. 10/949,799 is a continuation of application No. 09/619,290, filed on Jul. 19, 2000, now abandoned.
Application No. 09/619,290 is a continuation-in-part of application No. 09/358,116, filed on Jul. 21, 1999, now abandoned.
Application No. 09/358,116 is a continuation of application No. 08/741,945, filed on Oct. 31, 1996, now abandoned.
Application No. 08/741,945 is a continuation-in-part of application No. 08/321,400, filed on Oct. 11, 1994, now abandoned.

(51) Int. Cl. A61K 38/17; C07K 14/47; C12P 21/02; C12N 5/08

(52) U.S. Cl. 514/8; 435/69.1; 435/320.1; 435/325; 530/395; 536/23.5

(57) ABSTRACT

An isolated and purified glycoprotein and functional analogues thereof are disclosed. The glycoproteins are characterized by being expressed on at least primitive hematopoietic cells, and being a ligand for L-selectin. The binding activity of the ligand of the present invention to L-selectin is not sulfation-dependent and it is neither inhibited by anti-CD34 antibodies nor by MECA-79 monoclonal antibody and the ligand is resistant to O-sialoglycoprotein endopeptidase activity. Further, the present invention provides a method of performing an overlay adherence assay by using isolated cells or cell lines as a substrate. The cells are prepared as the substrate for the assay using a cytocentrifuge with a modified sample chamber allowing placement of the cytocentrifuged cell pellet to any selected location on the slide as required by the shear conditions employed for any given assay.

This application is a continuation of U.S. patent application Ser. No. 10/949,799, filed Sep. 24, 2004, which is a continuation of U.S. patent application Ser. No. 09/619,290, filed Jul. 19, 2000, which is a continuation-in-part of U.S. patent application Ser. No. 09/358,116, filed Jul. 21,1999, which is a continuation of U.S. patent application Ser. No. 08/741,945, filed Oct. 31, 1996, which is a continuation-in-part of U.S. patent application Ser. No. 08/321,400 filed Oct. 11,1994, all of which are incorporated herein by reference.

### BACKGROUND OF THE INVENTION

1. Technical Field

The present invention involves the development of compounds which can regulate and control the function of adhesion molecules as well as methods and apparatus for testing for adhesion molecules.

2. Background Art

Adhesion molecules are involved in the fundamental control of cell-cell interaction and cellular migration. Adhesion molecules regulate diverse processes in inflammation, hematopoiesis and tumor metastasis. (Woodruff, et al., 1987; Springer, et al., 1987; Sharon and Lis, 1993; Sackstein, 1993) For general reviews on adhesion molecules see Carlos and Harlan, 1994 and Chin et al., 1991. It would be useful to develop reagents which can control and regulate the adhesion proteins, particularly within the selectin family.

The peripheral lymph node “homing receptor”, L-selectin (CD62L), is a −75 kDa glycoprotein which mediates attachment of lymphocytes to lymph node (LN) high endothelial venules (HEV), an adhesive interaction which is the first step in the migration of lymphocytes from blood into lymphoid tissues (Gowans and Knight, 1964; Marchesi and Gowans, 1964). This trafficking of lymphocytes from blood into lymph nodes is markedly nonrandom and is initiated by specific adherence of the lymphocytes to HEV. L-selectin is the principal lymphocyte membrane glycoprotein mediating this attachment (see Shimizu and Shaw, 1993 and also U.S. Pat. No. 5,489,578 columns 3-4 for a review).

The L-selectin protein is recognized by a variety of monoclonal antibodies (mAbs) in humans (Gatenby et al., 1982 (Leu-8); Reinherz et al., 1982 (TQ-1); Tedder et al., 1990 (LAM)) and is a member of the selectin family of adhesion molecules, which includes P-selectin (CD62P) and E-selectin (CD62E). Selectins share a common structure consisting of an amino-terminal calcium-dependent lectin domain, an epidermal growth factor domain, a variable number of repeat sequences bearing homology to complement regulatory and catalytic proteins binding C3b or C4b, a transmembrane portion, and a C-terminal cytoplasmic tail (Bevilacqua and Nelson, 1993; Rosen, 1993). The molecular weight varies among leukocytes due to differences in posttranslational glycosylation among subsets of leukocytes (Carlos and Harlan, 1994). The lectin domain of these proteins directs their adhesion to carbohydrate molecules present on the cell surface.

The adhesive interaction between lymphocytes and HEV has been extensively analyzed using an in vitro binding assay (Stamper and Woodruff, 1976). The adhesive interaction between L-selectin and its ligand(s) was first identified by this in vitro binding assay in which lymphocytes are overlaid onto frozen sections of lymph nodes (Stamper and Woodruff, 1976). This “lymphocyte-HEV adherence assay” is an in vitro approximation of physiologic adhesion mediated by L-selectin and has been the conventional approach for studying the adhesive function of L-selectin in its native state on the surface of leukocytes. Indeed, in early studies, antibodies directed against L-selectin were operationally identified by their ability to block lymphocyte-HEV adherence in the assay (Rasmussen et al., 1985; Gallatin et al., 1983), and these antibodies subsequently led to the identification of L-selectin at a molecular level. This assay is performed under shear at 4° C., whereby binding mediated by L-selectin is maximized and effects of other adhesion molecules are minimized (Shaw et al, 1986; Spertini et al., 1991). Under these conditions, lymphocytes will adhere specifically to lymph node HEV via interactions between L-selectin and its corresponding ligand(s).

The interaction of L-selectin with its corresponding ligand(s) on HEV is calcium-dependent (Woodruff et al., 1977) and requires the presence of sialic acid (Rosen et al., 1985; True et al., 1990) and sulfate (Imai et al., 1993) on the ligand(s) (see U.S. Pat. No. 5,489,578 column 4 for a review). L-selectin behaves as a lectin and recognizes sialylated, high mannose residues on its corresponding endothelial ligands which are identified by the monoclonal antibody MECA-79 (Streeter, 1988; ATCC Accession number HB-9479; U.S. Pat. No. 5,403,919).

Most of what is known about L-selectin ligands has been derived from studies of molecules recognized by MECA-79 (Streeter et al, 1988; ATCC Accession Number HB-9479; U.S. Pat. No. 5,403,919), a rat anti-murine lymph node monoclonal antibody which identifies HEV by attaching to L-selectin ligands. In murine lymph nodes, MECA-79 and a murine L-selectin-Ig chimeric construct each immunoprecipitate several glycoproteins (Watson et al., 1990), two of which have been well-characterized: GLYCAM-1 (Imai et al., 1991), a secreted protein of 50,000 m.w., and CD34, an integral membrane protein of ˜90,000 m.w. (Baumhueter et al., 1993). In vitro adherence of lymphocytes via L-selectin can be inhibited by carbohydrates such as mannose-6-phosphate (man-6-P), PPME (Phosphomannan monoester core from Hansenula hostii, a phosphomannosyl-rich polysaccharide), and fucoidin (a sulfated, fucose-rich polysaccharide) (Stoolman and Rosen, 1983). In addition, the MECA-79 determinant is found on an HEV glycoprotein of 200,000 m.w. (Hemmerich et al., 1994) and on a selectively modified subset of the mucosal vascular addressin (MAdCAM-1) which supports L-selectin-mediated lymphocyte adhesion under shear (Berg et al., 1993). The HEV ligands recognized by MECA-79 are all heavily glycosylated mucin-like proteins (Shimizu and Shaw, 1993) rich in O-linked glycosylations bearing sialic acid. Treatment of endothelial L-selectin ligands with neuraminidase (Hemmerich et al., 1994; True et al., 1990) and with O-sialoglycoprotease abrogates the binding of L-selectin (Puri et al., 1995).

The molecular determinant recognized by both MECA-79 and L-selectin chimera is sulfation-dependent as demonstrated by markedly diminished ligand recognition following metabolic inhibition of sulfation by chlorate (Hemmerich et al., 1994). These results have highlighted the critical role of sulfation in ligand activity (Hemmerich et al., 1994; Imai et al., 1993), a conclusion supported by the fact that naturally-occurring unsulfated GLYCAM-1 is not capable of binding L-selectin (Dowbenko et al., 1993). Moreover, sulfation is characteristic of purified glycolipids (Suzuki et al., 1993), oligosaccharides (Greene et al., 1995) and glycosaminoglycans (Stoolman and Rosen, 1983) which bind to L-selectin, and the degree of sulfation correlates with binding affinity (Stoolman and Rosen, 1983).

Although present on endothelial cells in most tissues (Beschorner et al., 1985), CD34 is best known for its expression on the earliest multilineage colony-forming hematopoietic stem cells (Civin et al., 1984). Hematopoietic progenitor cells characteristically express both L-selectin and CD34 (Terstappen et al., 1992), and there is growing evidence that L-selectin plays a role in hematopoiesis (Terstappen et al., 1993; Kobayashi et al., 1994; Dercksen et al, 1995). The characterization of L-selectin and its ligands among progenitor cells is of considerable interest as adhesion proteins regulate cell-cell and cell-stromal interactions fundamental to hematopoiesis.

It would be useful to have strategies which would allow regulation of hematopoiesis since it is regulated by cell-cell and cell-stromal interactions. For example, Terstappen et al (1993) have shown that activation of L-selectin increases the clonogenic capacity of stem cells. Moreover, Dercksen et al (1995) have demonstrated that hematologic recovery following hematopoietic stem cell transplantation in humans correlates with the number of CD34+/L-selectin+ cells infused.

During recovery of immune function following bone marrow transplantation, pathologic changes have been observed following transplantation which interfere with lymphocyte migration and HEV integrity. Further, in addition to changes in lymph node structure, alterations in lymphocyte migration can occur secondary to the effect of pharmacologic agents used in posttransplant therapy such as corticosteroids (Sackstein, 1993). It would be useful to have an agent which can assist in reestablishing lymphocyte trafficking and so immune function following bone marrow transplantation.

The crucial role of adhesion molecules in controlling and directing the inflammatory process indicates that a reagent which interferes with the process, i.e., an anti-adhesive (see for example U.S. Pat. No. 5,489,578; 5,512,442; 5,304,640), could have anti-inflammatory properties.

Further, cell adhesion molecules are involved in metastasis, therefore it would be useful to develop an anti-adhesive which has anti-metastatic properties. In particular, with the identification of L-selectin on hematopoietic cells, it would be useful to have an anti-adhesive that affects L-selectin in leukemia to decrease the growth and spread of malignant hematopoietic cells throughout the body.

Further, it would be useful to have additional cell markers and monoclonal antibodies directed against these cell markers to allow for cell targeting.

In general, assays for determining the adhesion between cells based on shear require the use of frozen sections of the substrate or target cell (Stamper and Woodruff, 1976; Sackstein et al, 1988) as described hereinabove and a cell suspension overlaying the substrate. This conventional overlay adherence/binding assay has previously been used only with frozen tissue sections, with lymph nodes as a principal example, as the substrate. The frozen section has the advantage of being a predictable monolayer. A monolayer is required since the overlay adherence assay mimics the blood flow over a vessel wall.

It would be useful to have available isolated cells or cell lines as the substrate in the assay. This would enable the use of cell suspensions rather than tissue blocks, giving rise to more reproducible results as well as increasing the types of cells available as a target and reducing the need for surgical procedures for tissue removal.

Initially, applicants attempted to grow the cell lines directly on the glass slides. However, the assay did not produce reproducible results because a predictable, consistent monolayer of cells was not generated. If there is no consistent monolayer, i.e. if there is an uneven surface due to clumping etc. of the cells, then adherence artifacts are found.

Initial attempts to use preparations of single cell suspensions deposited on a glass slide utilizing a Cytospin 3 Cytocentrifuge (Shandon Lipshaw, Pittsburgh, Pa.) were also generally unsuccessful. Procedures using a cytocentrifuge placed a single layer cell pellet/substrate in the center of the slide (FIG. 11, 13, 15). When these slides were used in the assay on a rotating platform (e.g. 80 rpm rotation) in the adherence assay at 4° C. for 30 minutes the rotational shear forces placed the overlay cell suspension utilized (typically lymphocytes) at a variety of positions on the slide depending on the platformn itself, the platform angle and the rotation speed as well as other factors. In general, the overlay cell suspension was not localized over the center of the slide above the cytocentrifuged placed cell substrate. It would therefore be useful to be able to modify the placement of the cytocentrifuged cell pellet (to any selected location on the slide) so that it can be used as a substrate in an adherence assay dependent on the shear conditions employed for any given assay.

### SUMMARY OF THE INVENTION

According to the present invention, an isolated and purified glycoprotein and functional analogues thereof are disclosed. The glycoproteins are characterized by being expressed on at least primitive hematopoietic cells, and being a ligand for L-selectin. The binding of ligand to L-selectin is sulfation-independent and it is neither inhibited by anti-CD34 antibodies nor by MECA-79 monoclonal antibody and is resistant to O-sialoglycoprotein endopeptidase activity.

Further, the present invention provides a method of performing an overlay adherence assay by using isolated cells or cell lines as a substrate. The cells are prepared as the substrate for the assay using a cytocentrifuge with a modified sample chamber allowing placement of the cytocentrifuged cell pellet to any selected location on the slide determined by the shear conditions employed for any given assay.

### BRIEF DESCRIPTION OF THE DRAWINGS

Other advantages of the present invention will be readily appreciated as the same becomes better understood by reference to the following detailed description when considered in connection with the accompanying drawings wherein:

FIGS. 1A-B are photomicrographs of cytocentrifuge preparations of KG1a cells demonstrating adherence of lymphocytes (small dark dots), (A) Lymphocytes adhere to KG1a in the presence of CD45 or isotype control antibodies (Abs), (B) Lymphocyte binding assay in the presence of LAM1-3 antibody (anti-L-selectin);

FIGS. 2A-C are FACS profiles of lymphocytes used in the binding assay after incubation with (A) isotope-matched IgG control, (B) LAM1-3, or (C) anti-CD45 antibodies, followed by GAM-FITC, results shown are representative of three independent experiments;

FIGS. 3A-D are FACS histogram profiles of KG1a cells sorted by FACS prior to the binding assay into CD34+ (A,B) and CD34− (C,D) fractions using mAb HPCA-2PE, control sorted cell fraction (A,C) were restained with a FITC-conjugated isotype matched monoclonal antibody, experimental sorted cell fractions were restained for CD34 using mAb QBEND10-FITC with (B) positive (>90%) and (D) negative (<10%) for CD34, results shown are representative of three independent experiments;

FIG. 4 is a photomicrograph showing the results of lymphocyte adherence assay performed on the sorted cells, and no differences in lymphocyte adherence were evident among the CD34+ and CD34− populations, adherence to the CD34 negative fraction is shown;

FIGS. 5A-F are FACS profiles of COS-7 cells transfected with either CD34-pCDM8 (E,F) or pCDM8 (mock, C,D), then analyzed by FACS and compared to KG1a (A,B) for CD34 expression, antibodies used were isotype-matched IgG₁ control (ACE) and anti-CD34 mAb QBEND10 (BDF), lymphocytes did not adhere to CD34-transfected COS-7 cells, despite higher levels of CD34 expression as compared to KG1a cells;

FIGS. 6A-C wherein: (A-B) are FACS profiles of KG1a cells stained with IgM control antibody (A, top) or MECA-79 (B, bottom) followed by fluorescein isothiocyanate (FITC)-conjugated goat anti-rat IgM and FIG. 6 (C) is a photograph of a gel showing KG1a and murine lymph node (LN) metabolically radiolabeled with ³⁵SNa₂SO₄ and immunoprecipitated with MECA-79, murine anti-human CD43 as a positive control or respective isotype control antibodies;

FIGS. 7A-C are photographs of representative results of adherence assays using suspensions of rat thoracic duct lymphocytes overlaid onto cytocentrifuge preparations of KG1a cells of light microscopy (250′) of lymphocytes (dark circles) adhering to cytocentrifuge preparations of KG1a cells following incubations with RPMI containing buffer alone (A, top), immediately following neuraminidase treatment (B, middle), and neuraminidase treatment followed by twenty-four hour incubation in 10 mM sodium chlorate (C, bottom), note the absence of lymphocyte adherence immediately following neuraminidase treatment;

FIGS. 8A-C are graphs showing representative results of flow cytometric analysis of sialic acid-dependent epitope of CD43 demonstrating recovery of surface CD43 despite inhibition of sulfation by indirect immunofluorescence, wherein (A) KG1a control levels, (B) KG1a immediately following neuraminidase treatment, and (C) KG1a 24 hours following neuraminidase treatment, cells were cultured in the presence of 10 mM sodium chlorate, monoclonal antibody L60 was used as primary antibody and the secondary antibody was PE-conjugated;

FIGS. 9 A-B are photographs of gels wherein (A) shows the inhibition of sulfation by sodium chlorate demonstrated by immunoprecipitation/SDS PAGE analysis of ³⁵S—S0₄ incorporation into CD43 and (B) is an autoradiograph of SDS-PAGE analysis of total 35S—SO₄— radiolabelled and ³⁵S-methionine/cysteine-d protein in the presence (+) and absence (−) of chlorate, lanes contain equivalent amounts of lysate material obtained from identical numbers of cells cultured in the presence of respective radiolabels for the terminal eight hours of the 24 hour culture period, marked inhibition of sulfation is demonstrated among all sulfated proteins in the presence of chlorate, without significant effects on protein synthesis as shown by equivalent profiles of ³⁵S-methionine/cysteine-radiolabelled proteins, molecular weight markers in kDa are shown at the left of the Figure;

FIG. 10 is an exploded view of a holder assembly showing a modified sample chamber of the present invention;

FIG. 11 is a front view of an assembled holder assembly of the prior art;

FIG. 12 is a front view of an assembled holder assembly of the present invention showing the lateral and vertical offset of the modified sample chamber;

FIG. 13 is a top plan view of the assembled holder assembly as shown in FIG. 11;

FIG. 14 is a top plan view of the assembled holder assembly as shown in FIG. 12;

FIG. 15 is a side view of the assembled holder assembly as shown in FIG. 11;

FIG. 16 is a side view of the assembled holder assembly as shown in FIG. 12;

FIG. 17 is a front view of a slide marked for use in the present invention;

FIG. 18 is a front view of a holder assembly with a sample chamber of the present invention partially cut away to show cellular deposition at the position indicated on the slide in FIG. 17;

FIG. 19 is a flow cytometric analysis of P-selectin-Ig binding to PSGL-1 on KG1a cells following mocarhagin digestion;

FIG. 20 is a flow cytometric analysis of PSGL-1 levels on KG1a cells and KL60 cells;

FIG. 21 is a graph showing the L-selectin mediated lymphocytic adherance to PSGL-1 deficient KG1a cells;

FIG. 22 is a scheme of N-Glycan biosynthesis showing the assembly and processing;

FIGS. 23 A-C is a graph and photos showing the identification of HECA-452-reactive membrane proteins and enhanced E-selectin ligand activity on the human hematopoietic cell line, KG1a;

FIGS. 24 A-B are graphs showing the resolution of E-selectin ligands expressed on the cell line KG1a;

FIGS. 25 A-C are photos and graphs showing the HECA-452-reactive CD44 expressed on KG1a cells function as an E-selectin ligand;

FIGS. 26 A-B are photos showing that HECA-452-reactive CD44 from freshly isolated acute myelogenous leukemia cells of subtype M5 which function as an E-selectin ligand;

FIGS. 27 A-B are photos showing the differential expression and E-selectin ligand activity of HECA-452-reactive CD44; and

FIG. 28 is a photo showing the expression of α1,3 fucosyltransferase IV, α1,3 fucosyltransferase, and α2,3 sialyltransferase on chemotopoietic progenitor cell lines.

### DETAILED DESCRIPTION OF THE PREFERRED EMBODIMENT

The present invention provides an isolated and purified glycoprotein that is a hematopoietic cell E-selectin L-selectin ligand (HCELL) and functional analogues thereof.

The term analogue as used herein is defined as a HCELL variant (alternatively the terms alteration, amino acid sequence alteration, amino acid sequence variant, glycosylation variant can be used) with some differences in their amino acid sequences or glycosylation pattern as compared to the native sequence of the HCELL. Ordinarily, an analogue will be generally at least 70% homologous with the native HCELL over any portion that is functionally relevant. In more preferred embodiments the homology will be at least 80% and can approach 95% homology to the amino acid sequence of the protein segment of HCELL. The homology will extend over a region of contiguous amino acids. The amino acid sequence of an analogue may differ from that of the glycoprotein of the present invention when at least one residue is deleted, inserted or substituted.

Differences in glycosylation may be present between the analogue and the present invention (see U.S. Pat. No. 5,489,578 column 1, lines 34-67 for a review of carbohydrates in physiologically relevant recognition/adhesion). For glycoproteins, the post-translational sugar modification are, in some cases, the functional determinants of the molecule. The core protein sequence may be important in the spatial display of the sugars but may not contribute to the activity directly. Ordinarily, an analogue will be generally at least 70% homologous with the native HCELL over any portion that is functionally relevant. In more preferred embodiments the homology will be at least 80% and can approach 95% homology to the glycosylation pattern of the carbohydrate portion of HCELL. The molecular weight of the glycoprotein may vary between the analogue and the present invention due to carbohydrate differences.

Functionally relevant refers to the biological property of the molecule and in this context means an in vivo effector or antigenic function or activity that is directly or indirectly performed by a naturally occurring (native) HCELL. Effector functions include but are not limited to include receptor binding, any enzymatic activity or enzyme modulatory activity, any carrier binding activity, any hormonal activity, any activity in promoting or inhibiting adhesion of cells to extracellular matrix or cell surface molecules, or any structural role. The antigenic functions essentially mean the possession of an epitope or antigenic site that is capable of cross-reacting with antibodies raised against a naturally occurring HCELL. Biologically active HCELL analogues share an effector function of the native HCELL which may, but need not, in addition possess an antigenic function.

The glycoprotein HCELL has the following functional characteristics. It is expressed on at least primitive hematopoietic cells. The glycoprotein is a ligand for both E-selectin and L-selectin. The ligand binding to L-selectin is not inhibited by anti-CD34 antibodies and is not recognized by the MECA-79 monoclonal antibody. The glycoprotein exhibits sulfation-independent function. The glycoprotein maintains L-selectin ligand function following treatment with O-sialoglycoprotein endopeptidase. The glycoprotein is designated hereinafter as HCELL.

Further, HCELL, is a membrane associated glycoprotein and functions as an adhesion protein ligand. The glycoprotein mediates attachment of L-selectin expressing cells such as lymphocytes to hematopoietic cells including primitive hematopoietic cells.

HCELL was initially identified on KG1a and is similar to endothelial L-selectin ligands in that its activity is calcium-dependent, requires the presence of sialic acid for L-selectin binding, and it is a glycoprotein. However, HCELL differs in two fundamental ways from its endothelial counterparts as currently characterized, and represents a novel L-selectin ligand glycoprotein. Firstly, although both mouse and human lymphocytes bind to KG1a through an L-selectin-medicated interaction (Rosen, 1994), neither a murine L-selectin-Ig chimera (LEC-IgG) nor a human L-selectin-IgG chimera attach to KG1a cells, though these molecules recognize endothelial L-selectin ligands (Majdic, 1994).

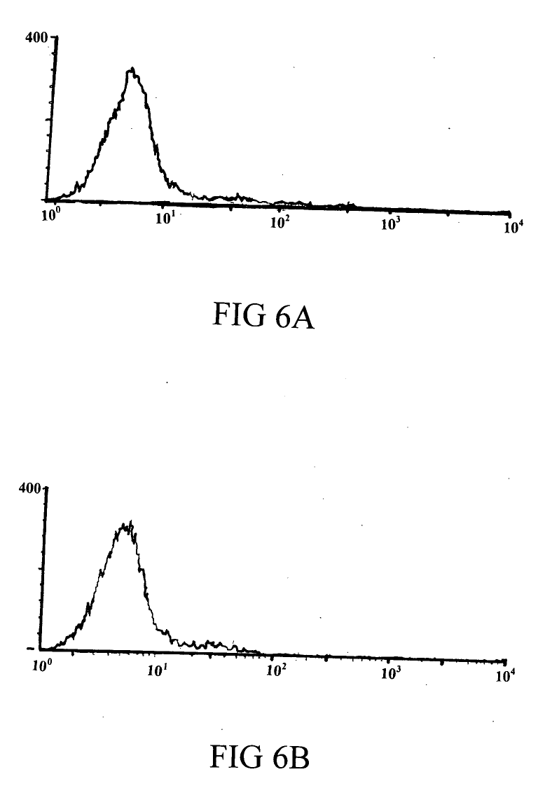

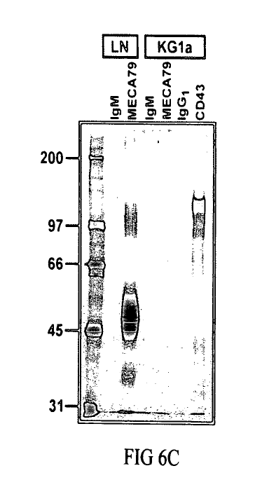

Secondly, the KG1a L-selectin ligand, HCELL, does not contain MECA-79-recognized epitopes. Both flow cytometric analysis of KG1a (HCELL) and immunoprecipitation studies of radiolabeled KG1a membrane proteins show no evidence of MECA-79 antigens on the surface of KG1a cells (FIGS. 6A, 6B), indicating that this epitope is not involved in the binding determinants of the KG1a L-selectin ligand (HCELL). Enzymatic cleavage studies and membrane stripping studies under a variety of pH and salt conditions indicate that the L-selectin ligand expressed on KG1a is a non-GPI-linked integral membrane glycoprotein.

Applicant asked whether the ligand of the present invention has the central structural features of other naturally-expressed L-selectin ligands, all of which have been characterized on endothelial cells. The role of sulfation in the binding activity of the KG1a L-selection ligand is at the core of this question, since it was shown in two previous “landmark” reports (Imai et al., 1993; Hemmerich et al., 1994) that the function of endothelial L-selectin ligands is sulfation-dependent. The Hemmerich et al. article in particular describing an L-selectin ligand, emphasized the functional role of sulfation for all endothelial L-selectin ligands, whereas the Imai et al. article focused only on the sulfation-dependent function of the secreted endothelial L-selectin ligand known as GlyCAM-1. Applicant's new data clearly distinguishes the L-selectin ligand of the present invention in two important ways: (1) it is not recognized by monoclonal antibody MECA-79, an antibody which recognizes the binding domain of all previously identified membrane L-selectin ligands, and (2) it does not require sulfation for binding to L-selectin. Thus, Applicant has unequivocally established that this ligand is novel—not only in the fact that it is expressed on a hematopoietic cell line but that it is structurally unique from all previously identified endothelial ligands.

Further, there is an independent relationship between P-selectin glycoprotein ligand-1 (PSGL-1) and the HCELL ligand activity showing that they are distinct molecules (Example 7).

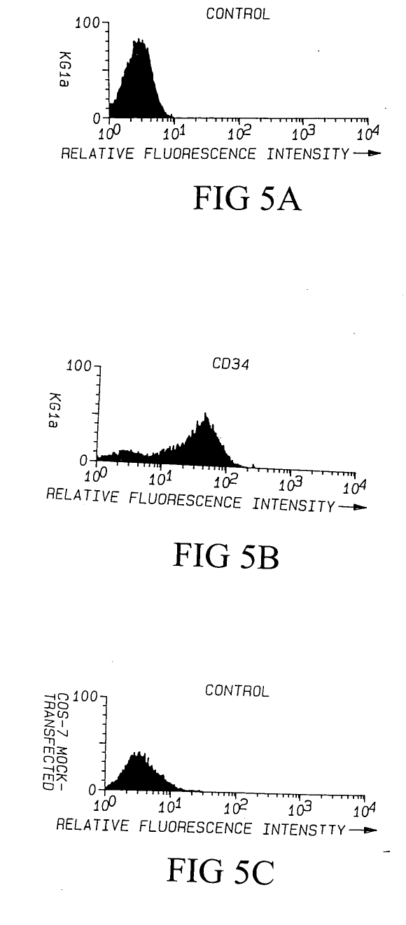

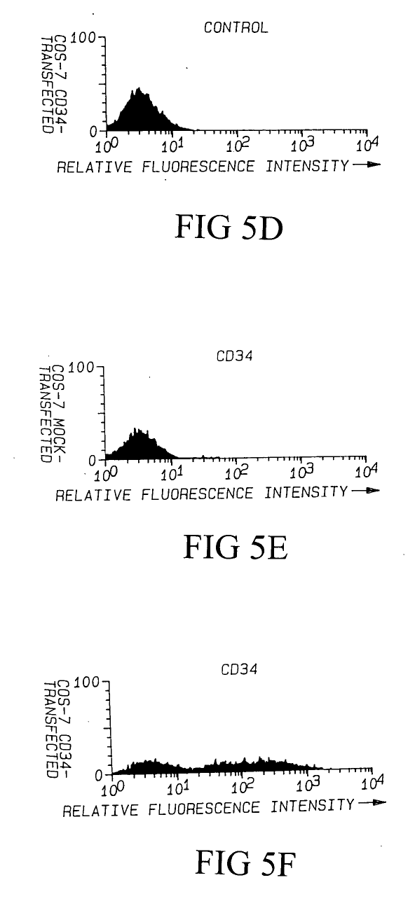

Since CD44 appears on multiple cell types including endothelial cells, the investigations were extended to evaluate the ability of CD44 expressed on human bone marrow endothelial cells (BMEC)(16) to engage in E-selectin ligand activity. As illustrated in FIG. 5B, immunoprecipitated BMEC CD44 was detectable by Hermes-1 moAb, however, BMEC CD44 was not HECA-452-reactive and did not possess any E-selectin ligand activity (FIG. 5A). These results confirmed that the expression of CD44, per se, did not confer E-selectin activity. CD44 functions as a scaffold for the appropriate post-translational glycosylations conferring E-selectin binding activity. Indeed, in an earlier study, it was noted that immunoprecipitated CD44 from human tonsils by Hermes-1 moAb did not exhibit E-selectin ligand activity (2). This study, along with these findings, emphasized the importance of appropriate post-translational modification for creating a bioactive E-selectin ligand.

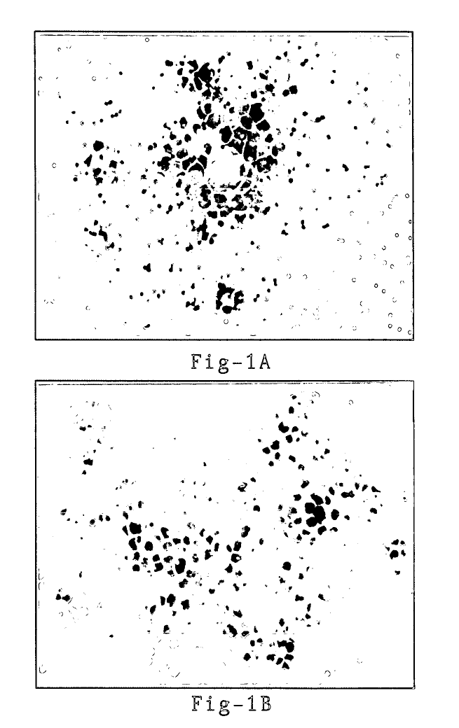

To analyze the role of glycosyltransferases required for HECA-452 antigen decoration, the inherent level of gene expression of α1,3 fucosyltransferases (FucTIV/FucTVII) and α2,3 sialyltransferases (ST2Gal III) was measured by RT-PCR in the human hematopoietic cell lines, K562, RPMI 8402, HL-60 and KG1a. Since the α1,3 fucosylation and α2,3 sialylation of carbohydrate binding determinants by FucTIV and FucTVII and ST3Gal III are critical for E-selectin ligand activity and synthesis of HECA-452 antigen (8, 25-29), it was suggested that these particular enzymes would be differentially expressed in the cell lines that were competent or devoid of E-selectin ligand activity. Moreover, since previous data demonstrated that the expression of CD44 on the leukemia cell lines, KG1a, HL-60 and RPMI-8402, was equivalent (>95% positive cell training by flow cytometry) (30) and E-selectin ligand activity was significantly higher on KG1a than on HL-60 cells or RPMI 8402 cells (FIG. 1C), this strongly suggested that post-translational modifications were crucial for HCEL activity of CD44. FucTIV expression was relatively similar in all cell lines, but the FucTVII expression was highest in HL-60 and KG1a cells (Lane 1, FucTIV, and Lane 2, FucTVII; FIG. 6A). ST3Gal III was expressed at a high level in KG1a cells and was expressed at a very low level in other cell lines (Lane 1; FIG. 6B). This observation highlighted the importance of α2,3 sialylation is a prerequisite for the biosynthesis of sialyl Lewis antigens that are thought to be related to the E-selectin carbohydrate binding determinants.

There have been many descriptions of human glycoconjugates that can serve as E-selectin ligands due to their affinity to human recombinant E-selectin chimeras (31-36), but, with exception of PSGL-1 and myloglycan (37), the identity of these molecules is unknown and their relevance as naturally expressed on the cell surface is not well understood. An E-selectin binding function of CD44 expressed on the human hematopoietic cell line, KG1a, and on freshly isolated de novo leukemias is described herein. This CD44 E-selectin ligand, designated as hematopoietic cell E-selectin ligand or HCEL, is dependent on glycosylations mediated by α1,3 fucosyl- and α2,3 sialyltransferases.

Just as the high expression of HECA-452 epitopes on PSGL-1 (CLA) on skin homing T-cells correlates with the capacity of PSGL-1 to function as an E-selectin ligand and the ability of these cells to traffic to the skin, the expression of HECA-452-reactive CD44 glycoform also correlates with its ability to function as E-selectin ligand. Of note, cutaneous infiltration of leukemia cells from a patient with an AML M5, which was analyzed in this study was observed grossly and by dermatopathologic examination of skin biopsies. The expression of HCEL on leukemia cells, therefore, offers a plausible explanation to the molecular pathophysiology of leukemia cutis. Similarly, since E-selectin is constitutively expressed on BMEC, HCEL this can facilitate normal or leukemic hematopoietic cell re-entry into the bone marrow, having broad implications as a biological surrogate for selection of HCEL+ and CD34+ hematopoietic cell populations for improving engraftment potential of an allogeniec bone marrow transplant and as a biological target for leukemotherapeutic exploitation.

The present invention also provides for an antibody directed against HCELL. The antibodies may be either monoclonal or polyclonal. Murine monoclonal antibodies are initially raised against KG1a cells. The monoclonals that are generated are then screened for the ability to block lymphocyte binding to KG1a. (See also U.S. Pat. No. 5,130,144 which discloses a method in which a monoclonal antibody is raised against KG1a cells.)

Utilizing these monoclonal antibodies, the HCELL is isolated by immunoprecipitation of KG1a membrane lysates as is standard in the art and used for the production of further antibodies as needed. Such methods can be found described Sambrook et al, Molecular Cloning: A Laboratory Manual, Cold Springs Harbor, N.Y., 1989, as well as additional methods of isolation and purification as are known in the art.

Additionally, the antibodies may be prepared against a synthetic peptide based on the sequence, or prepared recombinantly by cloning techniques or the natural gene product and/or portions thereof may be isolated and used as the immunogen. Such proteins or peptides can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering-A Practical Guide, W.H. Freeman and Co., 1992. Antibody fragments may also be prepared from the antibodies and include Fab, F(ab′)₂, and Fv by methods known to those skilled in the art.

For producing polyclonal antibodies a host, such as a rabbit or goat, is immunized with the protein or peptide fragment of the protein, generally with an adjuvant and, if necessary, coupled to a carrier; antibodies to the protein are collected from the sera.

For producing monoclonal antibodies the technique involves hyperimmunization of an appropriate donor with the protein or peptide fragment, generally a mouse, and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

The antibody or antibody fragment can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982.) The binding of antibodies to a solid support substrate is also well known in the art. (see for a general discussion Harlow & Lane Antibodies: A Laboratory Manual, Cold Spring Harbor Laboratory Publications, New York, 1988) The detectable moieties contemplated with the present invention can include, but are not limited to, fluorescent, metallic, enzymatic and radioactive markers such as biotin, gold, magnetic particles, ferritin, alkaline phosphatase, b-galactosidase, peroxidase, urease, fluorescein, rhodamine, tritium, ¹⁴C and iodination.

The method of targeting cells includes the steps of preparing antibodies directed against HCELL as described above and coupling the antibodies to the appropriate agent whether for cell killing, cell selection or cell identification. For cell killing, toxins such as ricin A chain, pseudomonas exotoxin A, diphtheria toxin, other plant and bacterial toxins as well as chemotherapeutic compounds can be coupled in the present invention forming an immunotoxin. For a general review of the antibody-toxin art see Ramakrishnan, 1990.

Cell targeting requires exposing a population of cells to the immunotoxin. A toxin bound antibody can be administered to the appropriate patient and targeted cells killed in vivo. The immunotoxin is administered and dosed in accordance with good medical practice, taking into account the clinical condition of the individual patient, the site and method of administration, scheduling of administration, and other factors known to medical practitioners. The “effective amount” for purposes herein is thus determined by such considerations as are known in the art. The amount must be effective to achieve improvement including but not limited to improved survival rate, more rapid recovery, or improvement or elimination of symptoms.

A pharmacological formulation of the antibody can be administered to the patient in an injectable formulation containing any compatible carrier, such as various vehicle, adjuvants, additives, and diluents; or the compounds utilized in the present invention can be administered parenterally to the patient in the form of slow-release subcutaneous implants of the targeted monoclonal antibodies, or iontophoretic, polymer matrices, liposomes, and microspheres can be employed. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Alternatively, cells can be removed from the patient and treated ex vivo selectively. For example, cells expressing HCELL can be removed through complement-mediated lysis from the ex vivo population and the remaining cells returned to the patient. Additional cell removal can be undertaken utilizing cell sorting, “panning” and magnetic bead separation. Alternatively, utilizing cell sorting, “panning”, magnetic bead separation and the like cell populations can be enriched for HCELL bearing cells and this enriched cell population returned to the patient if needed.

The targeted cells to be removed are cells expressing HCELL and can be selected from the group consisting of leukemic cells, malignant hemopoietic progenitor cells, or any malignant cell expressing the marker.

The present invention also provides a method of regulating hematopoiesis, particularly in reconstitution of the immune system following bone marrow transplantation. The present invention includes the steps of selecting those cells with high(+) or low(−) expression of HCELL depending on the growth characteristics associated with the marker density needed by the patient. The selection procedure utilizes ex vivo methods as described herein. After selection, the selected cell type is cultured in vitro, if needed, to expand the population using standard methods known in the art. The patient is then infused with the expanded, enriched HCELL+ or HCELL− population as needed.

The present invention further provides a method of regulating inflammatory response by interrupting cellular migration into lymph nodes and sites of both acute and chronic inflammation including the step of administering to the patient either functional analogues or antibody directed against HCELL thereby disturbing cellular migration mediated by HCELL by blocking the L-selectin-mediated cell attachment site and can be injected directly at the inflamed site if needed. The regulation of the inflammatory response would be useful in autoimmune disorders, post-ischemic tissue injury and sepsis (Carlos and Harlan, 1994). Administration and effective dose are as described for immunotoxins hereinabove.

See also U.S. Pat. Nos. 5,484,891; 5,489,578; 5,464,778; 5,304,640; 5,538,724 and 5,403,919 for further methods and discussion of treatments with selectin ligands, all incorporated by reference in their entirety.

As described herein the adhesive interaction between cells is analyzed using a modified in vitro binding assay (Stamper and Woodruff, 1976). The in vitro binding assay requires that a cellular suspension be overlaid onto frozen tissue sections (Stamper and Woodruff, 1976). The assay requires that the substrate be positioned such that the forces of the rotating platform will deposit the overlay cell suspension on top of the substrate.

The present invention provides for the use of isolated cells instead of frozen tissue sections as the substrate of the binding assay. The cells may be established cell lines or isolated cells from any source, mature or immature, including peripheral blood samples, from bone marrow aspirates, and from tissues donated through organ harvests or other sources as appropriate.

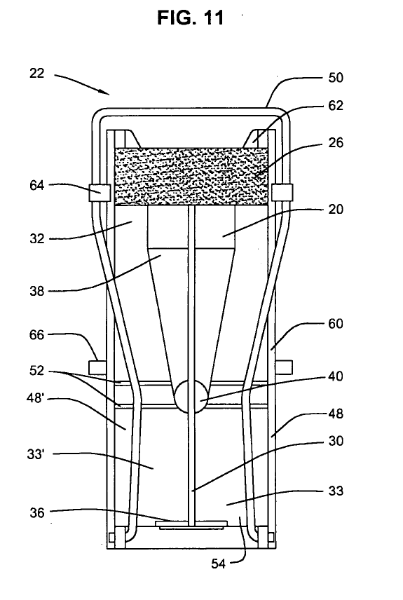

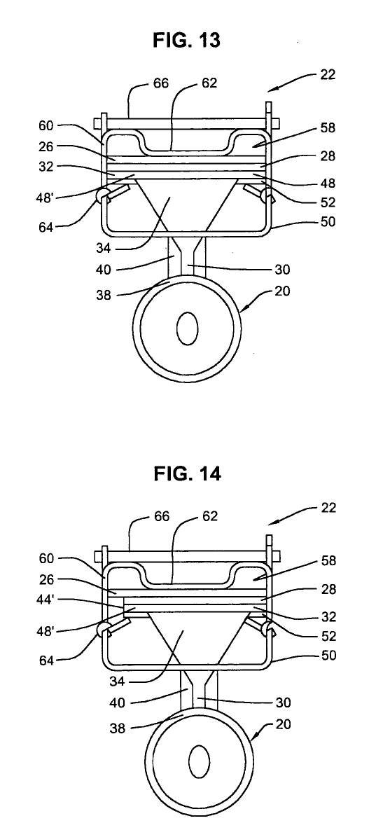

To prepare the cell substrate a protocol is used as further described in Example 1, which requires a modification of an existing sample chamber for use in a cytocentrifuge. When the prior art sample chamber as shown in FIGS. 11, 13 and 15 is used the cell substrate pellet from the cytocentrifuge is deposited in the middle of the slide to be used as the substrate. As described herein above this is not the preferred location for an overlay adherence assay. The preferred location must be determined for each rotation table and the substrate deposited at that site.

U.S. Pat. Nos. 4,391,710, 4,678,579 and 4,729,778 disclose the cytocentrifuge and holder assembly of the prior art. The design and structure of a cytocentrifuge and the general design and construction of the holder assembly and sample chamber are disclosed in these patents and are incorporated in their entirety by reference. The terms used in describing a preferred embodiment of a modified sample chamber of the present invention are the same as in these patents where possible.

The present invention allows the use of isolated cells or cell lines as a substrate, the cells being able to be deposited on a selected location of a slide which must be determined for the shear conditions employed for any given adherence assay as described herein above. The present invention provides a sample chamber assembly comprising a sample chamber including cell substrate receiving means for receiving a cell substrate and depositing means for placing the cell substrate on a slide surface during cytocentrifugation, connecting means for connecting together a slide and sample chamber and fixing the depositing means at one of a plurality of positions relative to the connecting means. The present invention further provides a method of making a sample chamber assembly by connecting together a slide and a sample chamber, the sample chamber including a cell substrate depositing port and fixing the cell substrate depositing port at one of a plurality of positions relative to the slide.

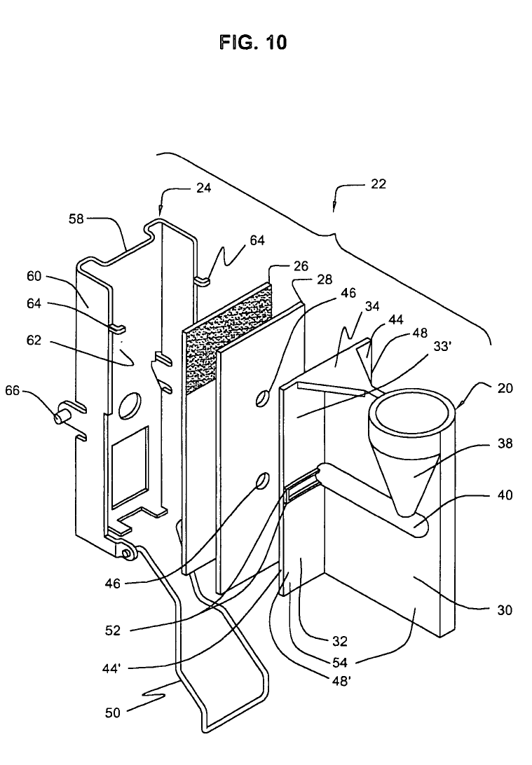

In an embodiment, a sample chamber, generally shown at 20, is shown in FIG. 10 which is an exploded view of a sample chamber assembly, generally shown at 22. The sample chamber assembly 22 includes a holding member generally shown at 24, a slide 26, a filter card 28 and the sample chamber 20. The holding member 24 is also referred to as a slide clip.

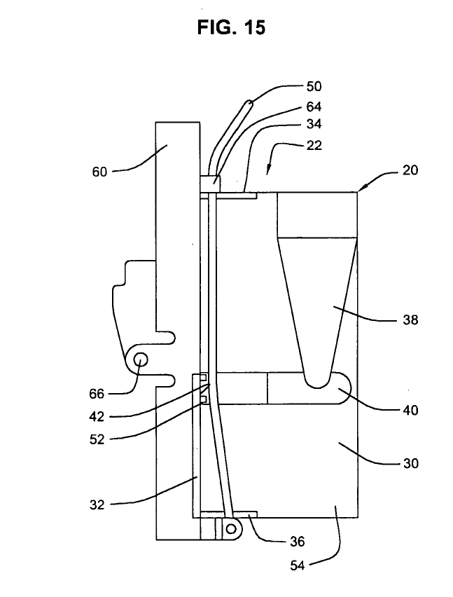

The sample chamber 20 generally comprises a flat, generally rectangular plate 30 having a rectangular end flange 32 disposed normal to and along an edge of the plate 30 such that the intersection of the rectangular plate divides the end flange into two side areas, 33,33′. The rectangular end flange 32 and plate 30 are reinforced and interconnected by a generally triangular upper end piece 34 as best seen in FIG. 10. In the prior art as shown in FIGS. 11 and 15 there is a lower end piece 36 opposite and spaced from the upper end piece 34, which is optionally present in the present invention. A vertical directed funnel 38 is contained within the plate 30 and interconnects with a horizontally extending discharge port 40 which extends from the funnel 38 within the plate 30 to the end flange 32. The discharge port 40 terminates at an opening 42 in the end flange 32. In the prior art sample chamber the distance between the opening 42 of the discharge port 40 and each of opposite long sides 44, 44′ of the rectangular end flange 32 is equidistant. In other words in the prior art the two side areas 33,33′ are generally the same size.

A filter card 28 is disposed between the end flange 32 and the slide 26. The filter card 28 is dimensioned to be coextensive with the end flange 32 and is provided with an opening 46 that is coextensive with the opening 42 of the discharge port 40. In an embodiment the filter card 28 may have two openings 42 positioned such that the filter card 28 may be disposed between the end flange 32 and slide 26 in either vertical orientation such that the filter card opening 46 will be coextensive with the opening 42 of the discharge port 40. During operation the filter card 28 will absorb the liquid content of the cell sample and the cells will be deposited on the slide 26. The filter card 28 can be separate as shown in FIG. 10 or can be formed as part of the sample chamber 20 as described in U.S. Pat. No. 4,678,579.

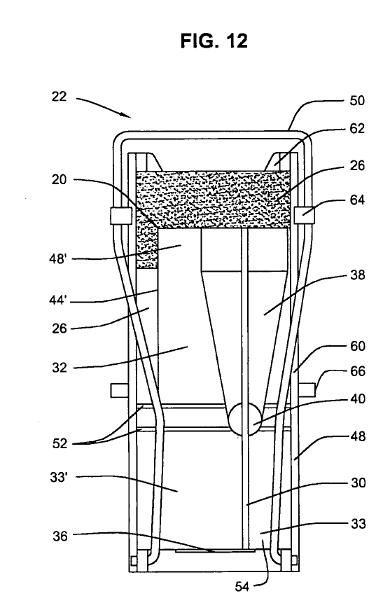

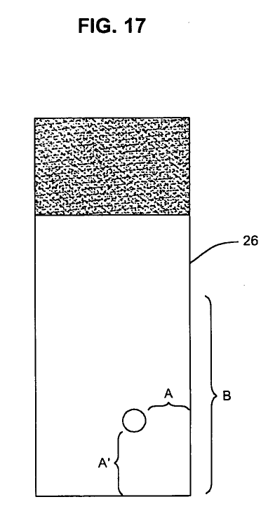

In the present invention the end flange 32 includes opposite lateral edge regions 48, 48′. The lateral edge regions 48, 48′ are disposed along each of the long sides 44, 44′ of the rectangular end flange 32. In the present invention one of the lateral edge regions 48 is removed as best shown in FIGS. 12, 14 and 18. The removal of one of the lateral edge regions 48 allows the lateral displacement or offset of the sample chamber 20 in the sample chamber assembly 22 as best shown in FIG. 12, 14 and 18. To allow removal of one of the lateral edge region 48 the lower end piece 36 may have to be removed. The lateral displacement is in the direction of the removed lateral edge region 48 removed. Further the amount of one of the lateral edge region 48 removed is generally such that the distance from the opening 42 of the discharge port 40 in the end flange 32 to the resulting long side 44 of the end flange 32 after removal of the lateral edge region 48 generally corresponds to A as shown in FIG. 17 as will be described and is no longer equidistant to the opposite long side 44′ of the rectangular end flange 32. In other words the two side areas 33,33′ are no longer the same size, and in this embodiment 33 is smaller than 33′. Alternatively, the lateral edge region 48′ can be removed. The filter card 28 is modified to be co-extensive with the end flange 32 after one of the lateral edge regions 48, 48′ is removed.

The holding member 24 includes a generally U-shaped clip 50 which pivots over the slide 26, filter card 28 and sample chamber 20 when they are positioned in the holding member 24 to hold them in position in the holding member 24. The front surface of the end flange 32 is provided with horizontally directed, slotted ledges 52 on opposite sides of the discharge port 40. The clip 50 when locked into position to hold the sample chamber assembly 22 together is disposed on the ledges 52 in the prior art as best. shown in FIGS. 11 and 15. In the present invention when the sample chamber 20 is moved laterally, the clip must be disposed on both sides of the discharge port 40 and generally on the ledges 52 as best shown in FIG. 12.

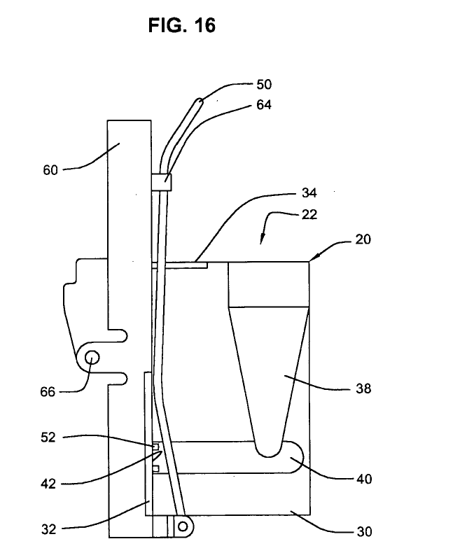

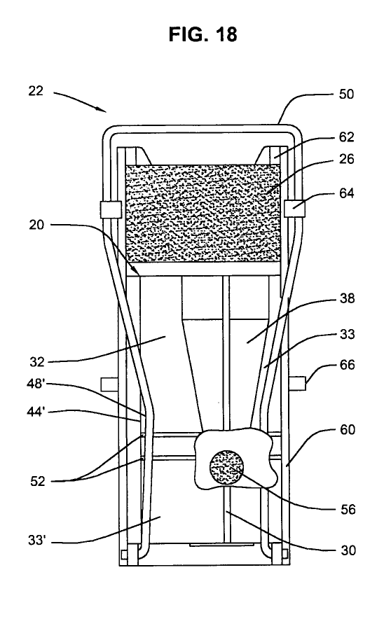

In the present invention the end flange 32 and rectangular plate 30 include a coextensive lower edge region 54. The lower edge region 54 is disposed along the lower short side of the rectangular end flange 32 and disposed along the lower edge of the rectangular plate 30 and spaced and parallel to the discharge port 40. In an embodiment of the present invention the lower edge region 54 is removed as best shown in FIGS. 16 and 18. The removal of the lower edge region 54 allows the vertical displacement or offset of the sample chamber 20 in the sample chamber assembly 22 as best shown in FIG. 16. Further the amount of the lower edge region 54 removed is generally such that the distance from the center of the discharge port opening 42 to the resulting lower edge after removal of one of the lower edge region 54 generally corresponds to A′ as shown in FIG. 17 as will be described.

In the present invention when the sample chamber 20 is displaced vertically, the clip 50 while disposed on both sides of the discharge port 40 will generally not be disposed on the ledges 52 as best shown in FIG. 16 but directly on the front surface of the end flange 32.

In an embodiment one of a lateral edge region 48, 48′ and a lower edge region 54 are removed allowing a combined vertical and lateral displacement of the sample chamber 20 as best shown for one embodiment in FIG. 18.

The modified sample chamber 20 can be made either by adapting existing prior art sample chambers formed in a plastic or other material which can be cut or shaved to size or the modified sample chamber 20 can be formed utilizing the methods disclosed in U.S. Pat. No. 4,391,710, 4,678,579 and 4,729,778 and methods known to those skilled in the art of plastic extrusion or forming in other materials. A series of modified sample chambers can be prepared having incremental changes in the amount of lateral edge regions 48, 48′, which opposite lateral edge region 48, 48′ is elected for modification and/or lower edge region 54 removed allowing for different A and A′ distances and prepared as a kit. The incrementally modified chambers are then available for testing to determine which incremental change will deposit the cell substrate at the proper position on the slide.

Alternatively, the holding member 24 can be adapted to allow displacement of the slide 26. The holding member 24 is comprised of a channel 58, which can be U-shaped or M-shaped, having opposite side plates 60, a common web 62 interconnecting the opposite side plates 60 and a pivot rod 66 for interconnecting to the centrifuge. A retaining hook 64 is disposed on each side plate 60 to retain the clip 50 in the locked position. To adapt the holding member 24, one of the side plates 60 can be bent outwardly such that the slide 26 can be displaced laterally while the sample chamber 20 is either not moved or is moved oppositely laterally and/or vertically. This allows for maximum freedom of positioning of the cell pellet 56 on the slide 26. In adapting the side plate 60, care must be taken to insure that the retaining hook 64 can still be used.

The depositing means include the discharge port 40 and the discharge port opening 42 through which the cell substrate pellet 46 is deposited on the slide 26 at one of a plurality of positions. The depositing means can further include the filter card 28 and filter card opening 46 which is coextensive with the discharge port opening 42.

The receiving means include the funnel 38 interconnected with the discharge port 40 such that under cytocentrifugation a cell substrate 56 disposed in the funnel 38 enters the discharge port 40.

The connecting means comprise the holding member 24, end flange 32 and rectangular plate 30 as well as the lateral edge regions 48,48′, side areas 33,33′ of the end flange 32 and side plates 60 of the holding member 24.

The modified sample chamber 20 (and/or holding member 24) allows for the deposition of a cell substrate 56 at a specified reproducible location on a slide. The location is chosen such that the cell pellet 56 deposited by the modified sample chamber 20 can function as a substrate in an overlay assay.

The determination of the point on the slide on which to deposit the cell substrate is determined empirically as it will differ slightly for each individual rotating platform (rotating table, for example a Labline Orbit Shaker) due to mechanical differences as well as for the cell types, cell density, slide angle, rotational speed, temperature and other factors known to those skilled in the art. The determination can be made by depositing a test cell suspension on a slide that is on the rotating platform. The slide is then observed under the adherence assay test conditions and the area where the cells aggregate is identified. The distances A and A′ and the distance B from the center of the slide is determined for the area of cell aggregation as shown in FIG. 17. A sample chamber 20 is then modified as described herein above to deposit the cytocentrifuge cell pellet substrate 56 at the calculated position as shown in FIG. 18. Alternatively, a series of incrementally modified sample chambers 20 can be used to establish test slides and the modified sample chamber 20 that provides a cytocentrifuge cell pellet substrate 56 with the most appropriate positioning under the shear conditions established for the given adherence assay can be used. In Example 1 a protocol for an assay to test L-selectin ligand adhesion interactions is described, however other adherence assays requiring shear forces can also be undertaken with the present invention.

Selectins are involved in a variety of adhesive interactions, with biological roles ranging from acute inflammation (regulation of leukocyte trafficking) to immune responses (activation of T cells and regulation of lymphocyte migration to lymphoid tissues) to hematopoiesis. Studies by applicant on the interaction of L-selectin and ligands on hematopoietic cells which function as an adhesive receptor-ligand pair, i.e. in vitro binding studies of lymphocytes to KG1a, a primitive CD34-positive human cell line derived from an acute myeloid leukemia (Civin et al., 1994; Koeffler et al., 1980) lead to the present invention. These studies surprisingly revealed highly specific adherence of lymphocytes to KG1a cells mediated by L-selectin on the lymphocyte, but unexpectedly not involving CD34 as the corresponding ligand as had been previously reported (Baumhueter et al, 1993). The results indicated the presence of a ligand, designated HCELL (the ligand has also been referred to as hematopoietic cell L-selectin ligand, HLL), for L-selectin on the surface of this hematopoietic progenitor cell line and provide the first evidence of sulfation-independent L-selectin-mediated adhesion between lymphocytes and a non-endothelial cell type.

For these studies, the lymphocyte-HEV adherence assay was used which is an in vitro approximation of physiologic adhesion mediated by L-selectin was used and as discussed herein above is the standard assay for determining adhesion. This assay required the functional interaction of respective membrane structures localized within natural cell ligand bilayers. It has been a fundamental tool in studying the function of L-selectin in its native state on the surface of lymphocytes. This binding assay was novelly adapted as described herein (FIGS. 10-18) to examine lymphocyte-hematopoietic cell adhesion, and the results provide the unexpected results of L-selectin-dependent adhesive interactions between lymphocytes and non-endothelial, hematopoietic cells.

Blood cell formation depends critically upon discrete cell-cell and cell-matrix adhesive receptor/ligand interactions which create specialized bone marrow microenvironments wherein cells proliferate and mature. L-selectin is characteristically expressed on a subpopulation of bone marrow cells which includes the earliest multilineage hematopoietic stem cells. Evidence that L-selectin may function in hematopoiesis has been obtained from both in vitro clonogenic assays (Koenig et al., 1994) and from clinical engraftment data following bone marrow transplantation (Dercksen et al., 1995).

L-selectin ligands have been recognized heretofore only on endothelial cells and the function of all have been shown to be sulfation dependent (see U.S. Pat. No. 5,489,578). The detection of a sulfation-independent L-selectin ligand on a non-endothelial cell (Example 6) expands the physiologic implications of L-selectin function beyond its well-characterized role in regulating leukocyte trafficking.

The adaptation of the assay allowed for the first time the use of cell lines in a lymphocyte-HEV adherence assay. In this assay, slides were prepared utilizing a modified cytocentrifugation apparatus (FIGS. 10-18) of KG1a cell suspensions which were used in place of slides of frozen lymph node sections as taught by the prior art. The KG1a cells were placed on the slides by using cytocentrifugation as further described hereinbelow in Example 1.

Several independent lines of evidence indicate that lymphocyte binding to KG1a is mediated primarily, if not solely, by L-selectin. First, an anti-L-selectin mAb (LAM1-3) previously shown to block L-selectin-mediated adherence to LN HEV (Spertini et al., 1991), completely inhibited PBL from binding to KG1a or LN HEV, whereas anti-CD45 and isotype control antibodies did not block lymphocyte binding. Second, L-selectin-mediated binding is a calcium-dependent event, and lymphocytes were unable to bind to KG1a in the presence of the calcium chelator EDTA. Third, carbohydrates such as man-6-P, PPME, and fucoidin inhibited lymphocyte adherence to KG1a. These compounds are all known to bind to L-selectin and to inhibit lymphocyte binding to HEV in the in vitro assay (Stoolman and Rosen, 1983; Stoolman et al., 1984). Lastly, it is known that PMA treatment of lymphocytes causes shedding of membrane L-selectin via a protein kinase C activation pathway, and corresponds to the loss of lymphocyte binding to LN HEV in the in vitro assay (Tedder et al., 1990). In these studies, PMA-treated PBL were no longer able to bind to KG1a.

These results revealed that KG1a possess a high affinity L-selectin ligand, however, a significant role for CD34 in this ligand activity was excluded by the following observations: (1) treatment of KG1a cells with a variety of anti-CD34 monoclonal antibodies, singly and in combination, had no effect on binding; (2) the CD34-positive human hematopoietic cell lines RMPI 8402 and MO7e did not support lymphocyte adherence despite higher levels of CD34 expression than that of KG1a; and (3) KG1a cells separated into subpopulations expressing high and low levels of CD34 showed equivalent binding (Oxley and Sackstein, 1994b). Treatment of KG1a with the enzymes neuraminidase, chymotrypsin, and bromelain abrogated lymphocyte binding to the cells, indicating that the ligand is a glycoprotein and that expression of ligand sialic acid is critical for function (Oxley and Sackstein, 1 994b).

The nature of the ligand was investigated by determining the effects of various enzyme treatments of KG1a on the binding capacity. Previous studies have shown that ligand expression of sialic acid is essential for L-selectin-mediated binding of lymphocytes to LN HEV (Rosen et al., 1985). In the present examples, neuraminidase-treated KG1a showed a complete loss of lymphocyte binding, indicating that sialic acid residues are also a necessary component on the KG1a L-selectin ligand; as such, lymphocyte adherence to KG1a involves carbohydrate motifs and is not based strictly on protein-protein interactions. This finding, combined with the results of protease experiments, indicates that the KG1a ligand is a glycoprotein.

To examine whether O-linked glycosylations on the ligand play a central role in the adhesive interaction, KG1a were digested with the enzyme O-sialoglycoprotein endopeptidase which specifically cleaves proteins at sites of O-linked sialo-glycosylation (Abdullah et al., 1992) and which has been shown to differentially cleave epitopes of the CD34 molecule (Sutherland et al., 1992). The data revealed that treatment of KG1a in suspension with the enzyme actively destroyed CD34 epitopes, yet had no effect on lymphocyte adherence. These results suggest that ligand sialic acid residues critical to binding are present on N-linked rather than on O-linked glycosylations.

The functional dependence on intact sialic acid moieties is consistent with results from endothelial L-selectin ligands (True et al., 1990) and suggested that epitopes critical for binding might be structurally conserved among these ligands. However, treatment of KG1a with O-sialoglycoprotease resulted in characteristic loss of enzyme-sensitive epitopes from CD34 (Sutherland et al., 1992) but did not affect adherence (Oxley and Sackstein, 1994b). Since the ligand activity of endothelial CD34 is eliminated by this treatment (Puri et al., 1995), this result provided further evidence that the KG1a ligand activity was not attributable to CD34, and, more generally, that the binding determinant of the ligand differs structurally from proteins recognized by MECA-79.

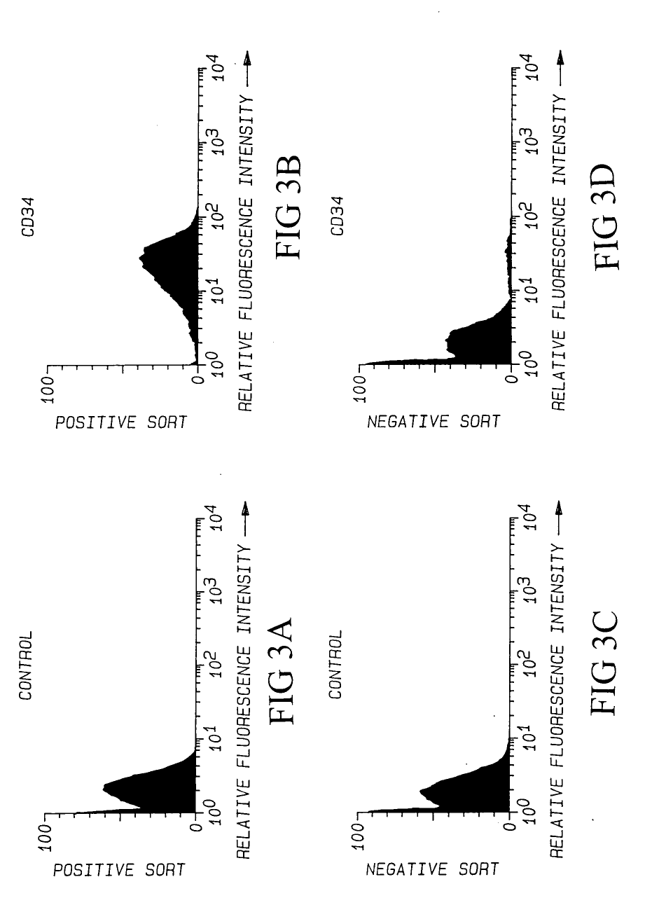

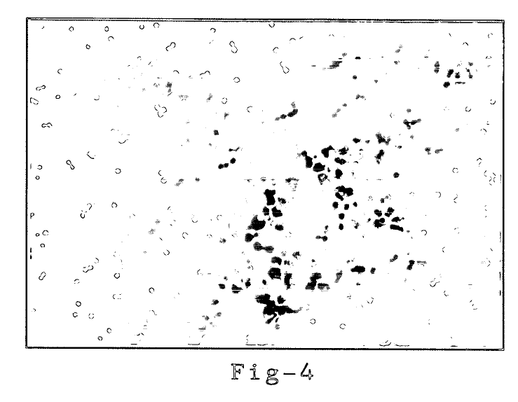

CD34 has been reported to be a ligand for L-selectin based on the finding that a murine L-selectin-IgG chimera molecule precipitated CD34 from a murine lymph node lysate (Baumhueter et al., 1993). The results as set forth in the examples indicate that CD34 as expressed on KG1a is not a functional ligand for lymphocyte L-selectin, as no difference in lymphocyte binding to sorted CD34− and CD34+ KG1a cells was observed (see FIGS. 3 and 4). Titration studies using varying proportions of KG1a and HL60 have demonstrated that the amount of lymphocyte adherence is directly proportional to the percentage of input KG1a cells, indicating that differences in lymphocyte binding to the positive and negative sorted fractions would have been evident if CD34 were the ligand. It is unlikely that a particular binding epitope of CD34 as selected, as this experiment was done using two different anti-CD34 mAbs to sort the KG1a. Two forms of CD34 on KG1a have been reported (“truncated” and “full length”) (Krause et al., 1993); however, these differences do not account for the data herein as sorting was also performed using QBEND10, which recognizes both forms.

In addition to sorting experiments, evidence that CD34 is not the L-selectin ligand on KG1a is derived from mAb blocking studies and adherence assays using other CD34 positive cells. None of the anti-CD34 mAbs tested, or any combination thereof, was able to block lymphocyte binding to KG1a. Furthermore, lymphocytes did not adhere to another primitive CD34+ cell line, RPMI 8402, and transfection of CD34 into COS-7 cells did not confer lymphocyte binding capacity. While potential glycosylation differences of the CD34 molecule expressed by these cell types could affect their ability to support lymphocyte adherence, this explanation is unlikely in light of equivalent adherence observed among the sorted CD34+ and CD34− KG1a cells. Taken together, the data presented here indicate that the CD34 glycoform present on hematopoietic cells is not a ligand for L-selectin. Moreover, flow cytometric analysis of the various cell lines utilized in the binding assay provides evidence that membrane structures such as LFA-1, VLA-4, CD44, Sialyl Le^{x} and CD43 do not play a primary role in lymphocyte adherence to KG1a since each of these molecules were also present on at least one other cell line tested that did not demonstrate lymphocyte binding.

HCELL is not recognized by MECA-79 monoclonal antibody (Example 5) which identifies L-selectin ligands on lymph node HEV. Immunofluorescence analysis of KG1a using MECA-79 shows no evidence of the protein identified by MECA-79. HCELL is shown to be unique from L-selectin ligands thus far identified.

The relationship of the KG1a ligand to proteins identified by MECA-79 were evaluated by performing adherence studies in the presence of MECA-79, flow cytometric analysis of MECA-79 antigens on KG1a, and immunoprecipitation studies of metabolically radiolabeled KG1a and lymph node organ cultures. Incubation of KG1a with MECA-79 did not interfere with lymphocyte adherence in the binding assay and flow cytometric studies showed no evidence of MECA-79 antigen on KG1a (FIG. 6A-B). Moreover, no MECA-79-immunoprecipitable proteins were detected from lysates of metabolically radiolabeled KG1a cells using either ³⁵SO₄ (FIG. 6C) or ³⁵S-methionine/cysteine, though characteristic bands were obtained from respectively radiolabeled murine lymph nodes (FIG. 6C). The absence of MECA-79-precipitable protein within KG1a lysates indicates that there are no intracellular accumulations of MECA-79 antigen, and, furthermore, that the MECA-79 antigen is not synthesized by the KG1a cells.

Direct cell-cell interactions were utilized to detect the presence of an L-selectin ligand on a hematopoietic cell. Other studies directed at identifying L-selectin ligands have relied on molecular approaches utilizing a murine L-selectin-IgG chimera molecule, synthesized in a human embryonal kidney cell line, as a probe (Watson et al., 1990). Of note, studies utilizing this chimera have failed to demonstrate binding of the molecule to KG1a cells (Majdic et al., 1994). In general, tissue- and species-specific patterns of glycosylations are well described, (Yamashita et al., 1983; Cullen et al., 1981; Yamashita et al., 1985) and such differences can affect the biological activity of proteins expressed in different cells (Cowing 1983; Huff et al., 1983). As it is known that glycosylation of L-selectin varies among different cells expressing the protein (Lewinsohn et al., 1987, Ord et al., 1990; Griffin et al., 1990), such differences may account for the observation here that native L-selectin, expressed on lymphocyte membranes, selectively binds to a corresponding ligand on KG1a cells while the chimera apparently does not. Similarly, differences in glycosylation of CD34 among endothelial cells and hematopoietic cells may account for the differential capacity of this protein to participate in L-selectin interactions among these cell types.

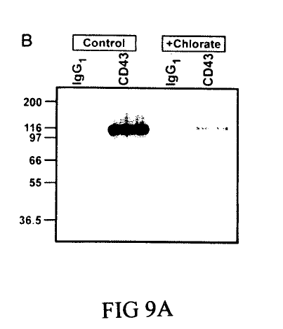

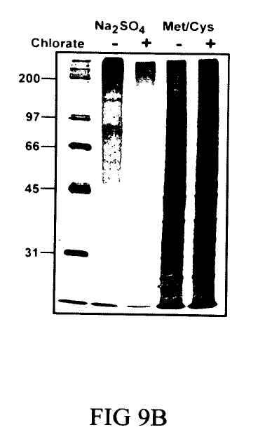

Recognition of endothelial ligands by MECA-79 is sulfation-dependent, however, absence of MECA-79 reactivity does not in itself indicate that sulfation does not participate in the binding determinant of the KG1a ligand of the present invention. Applicant directly examined this by measuring ligand activity following incubation with chlorate (Example 6), a highly efficient metabolic inhibitor of both saccharide and peptide sulfation (Baeuerle and Huttner, 1986). The results of chlorate incubation on L-selectin ligand activity of KG1a was evaluated and the results are shown herein below (Table 3 and FIG. 9). Incubation of KG1a in chlorate-containing media was performed following complete desialylation of the cells by neuraminidase treatment. Initial studies of the kinetics of recovery of binding activity following either neuraminidase treatment or protease digestion of membrane protein showed absence of ligand activity for twelve hours; thereafter, ligand activity increased in a linear fashion with return to baseline levels within twenty-four hours. Return of binding activity following desialylation or protease treatment was blocked by metabolic inhibition of N-linked glycosylation by tunicamycin treatment, suggesting that re-expression of activity results predominantly from synthesis/processing of nascent ligand and not from transport to membrane of stored mature protein (Table 3).

Accordingly, KGI a were incubated in chlorate for twenty-four hours to inhibit sulfation throughout the duration of ligand reexpression. ³⁵SO₄-pulse radiolabeling studies at eight hour time intervals within the twenty-four hours of chlorate incubation indicated that sulfation was inhibited throughout the entire incubation period, as demonstrated by trichloroacetic acid-precipitable radiolabeled protein counts (chlorate-incubated counts consistently <10% that of non-chlorate treated controls) and SDS-PAGE analysis of radiolabeled protein of cell lysates. As shown in Table 3, the binding activity of the KG1a ligand was unaffected by chlorate incubation, though inhibition of sulfation was confirmed, even for the terminal six hours of the incubation period, by a marked reduction of ³⁵SO₄ incorporation into CD43, a sulfated sialomucin (Wilson and Rider, 1992) expressed on KG1a (Oxley and Sackstein, 1994b) (see FIG. 9). The data shows that sulfation, regardless of whether it may be present on protein or sugar, is not critical to the functional interaction with L-selectin. These data, together with the lack of MECA-79 reactivity, indicate that sulfation does not contribute significantly to the function of the KG1a ligand.

The data presented herein expands the understanding of the structure of selectin ligands and provides the first evidence of a functional, membrane glycoprotein L-selectin or E-selectin ligand whose binding activity is not sulfate-dependent. Multiple studies have emphasized the importance of sulfation in the binding determinant of ligands for both L-selectin (Hemmerich et al., 1994; Imai et al., 1993; Suzuki et al., 1993; Green et al., 1995; Stoolman and Rosen, 1983; Green et al., 1992; Hemmerich and Rosen, 1994b) and P-selectin (Aruffo et al., 1991; Sako et al., 1995; Pouyani et al., 1995), and all previously identified naturally-occurring membrane ligands for these selectins bear sulfate-dependent activity. The contribution of sulfate modifications may relate to the localization of negative ions, on either sugar (Hemmerich and Rosen, 1994b) or protein (Sako et al., 1995; Pouyani, 1995; Niehrs et al., 1992) determinants. Such a role for charge is supported by the finding that unsulfated anionic polysaccharides such as polymers of phosphated mannose can bind to L-selectin (Yednock et al., 1987). Within the KG1a ligand, it is possible that glycosidic and/or amino acid modifications such as phosphorylation, or the molecular composition of the discrete sugars or amino acids comprising the binding domain, create a relevant anionic milieu. Although the precise structural features which direct binding activity for this and other L-selectin ligands remain to be determined, that data indicate that cell membrane binding determinants conferring high affinity recognition of L-selectin are not strictly conserved: while sulfated glycoprotein ligands may be characteristic of endothelial cells from lymphoid tissues, non-sulfated ligand forms may direct L-selectin- or E-selectin-specific cell-cell recognition and adhesion events in other cell types, be it non-endothelial cells or non-lymphoid endothelium.

Also provided by-the present invention is a method of determining a pharmaceutical use by modifying an L-selectin or E-selectin activity of a cell line and then applying pharmaceuticals to the cell line which effect the modified cell line selectin activity. These pharmaceuticals can include any pharmaceuticals known to those of skill in the art. Additionally, by “affect” it is meant that the pharmaceutical can upregulate or suppress selectin activity. In the preferred embodiment, the selectin which is being affected is the HCELL, which is disclosed above.

The above discussion provides a factual basis for the characterization and use of HCELL and the method and apparatus for performing adherence assays. The methods used with and the utility of the present invention can be shown by the following examples.

### EXAMPLES

General Methods:

Cell Lines. Cell lines used in these studies were obtained from the following sources: KG1a and Nalm 16, gift of Dr. William E. Janssen; HL60, K562, and Raji, gift of Dr. Lynn Moscinski; COS-7, gift of Dr. Kenneth Zukerman (all from H. Lee Moffitt Cancer Center, Tampa, Fla.); RPMI 8402, gift of Dr. Daniel G. Tenen (Harvard Medical School, Boston, Mass.). All cells were cultured in RPMI 1640 (Gibco-BRL, Gaithersburg, Md.) supplemented with 10% heat-inactivated fetal bovine serum (FBS) in a humidified chamber at 37° C. with 5% CO₂ in air.

MECA-79 antibody. MECA-79 antibody was a gift from Dr. Phillip Streeter, Searle Research Laboratories/Monsanto Co., St. Louis, Mo.). A MECA-79 hybridoma is available from American Type Culture Collection (ATCC), 12301 Parklawn Drive, Rockville, Md. 20852 as accession number HB-9479. The production of the MECA-79 hybridoma is described in U.S. Pat. No. 5,403,919.

Preparation of Lymphocytes. Human peripheral blood lymphocytes (PBL) were isolated by Ficoll density gradient from blood drawn in sodium citrate. To obtain rat thoracic duct lymphocytes (TDL), thoracic ducts of rats were cannulated as described by Bollman et al.(1948). Lymph was collected in phosphate buffered saline (PBS) with 0.1% penicillin/streptomycin and 5 U/ml heparin. PBL or TDL were washed three times in RPMI 1640 medium without bicarbonate (Gibco-BRL), pH 7.4, and suspended at 1′ 10⁷ cells/ml in above medium with 5% FBS and kept on ice until use in the adherence assay.

Lymphocyte Adherence Assay. The procedure for the in vitro binding of human or rat lymphocytes to KG1a was adapted from the rat lymphocyte-lymph node binding assay which has been described by Stamper and Woodruff (1976) and Sackstein et al. (1988) and described in Example 1. Cytocentrifuge preparations of KG1a or other cell lines were made on a Cytospin 3 Cytocentrifuge (Shandon Lipshaw, Pittsburgh, Pa.). Frozen rat LN sections 8 mm thick were mounted on slides, and lymphocyte binding to LN HEV served as a positive control in all experiments. Slides were air dried, fixed in 3% glutaraldehyde (Electron Microscopy Sciences, Fort Washington, Pa.) in PBS, rinsed with PBS, incubated in 0.2M L-lysine (Sigma Chemical Company, St. Louis, Mo.) to block unreacted glutaraldehyde, then rinsed and held in RPMI 1640 with 1% FBS at 4° C. until use in experiments.

Lymphocyte suspensions (200 ml) were overlaid onto cytocentrifuge or LN sections in duplicate and placed on a rotating platform (80 rpm) at 4° C. for 30 minutes. Slides were then rinsed in cold PBS to remove non-adherent lymphocytes, fixed in 3% glutaraldehyde, and stained with methyl green-thionin. Slides were examined under the light microscope for adherence of lymphocytes to KG1a or LN HEV.

Number of lymphocytes adherent to confluent area of KG1a were counted by light microscopy using an ocular grid under 250′ magnification. Quantitation was performed by examining two fields per slide, minimum of two slides per experiment, three separate experiments. Results are presented as % binding compared to corresponding untreated control sections.

Treatment of Lymphocytes with Potential Inhibitors. Lymphocytes in RPMI 1640 medium with 5% FBS were pre-incubated (30 minutes on ice) and the assay performed in the presence of the following inhibitors: 1 mM EDTA (no pre-incubation period); 10 mM D-mannose-6-phosphate (Sigma); 10 mg/ml PPME (kindly provided by Dr. M. E. Slodki, USDA, Peoria, Ill.); and 5 mg/ml fucoidin (Sigma).

Antibody Blocking Experiments. Lymphocytes (1′ 10⁷ cells/ml) were pre-incubated on ice for twenty minutes with mAbs at 1.0 mg/mi and used in the binding assay without further washing. The following mAbs were used: LAM1-3 (anti-L-selectin) (kind gift of Dr. Thomas Tedder, Duke University, Durham, N.C., and also obtained from Coulter Corp., Hialeah, Fla.); anti-CD45 (leukocyte Common Antigen) (Becton Dickinson, San Jose, Calif.); and IgG₁ (isotype control) (Coulter). In some experiments, prepared KG1a slides were incubated with 0.2 mg of anti-CD34 antibodies {HCPAa-1 (clone My10) and HPCA-2 (clone 8G12) (Becton Dickinson), QBEND10 (AMAC) and 12.8 (kindly provided by Dr. Pat Roth, Coulter Corp.)} in RPMI 1640 with 5% FBS for 30 minutes prior to the binding assay.

PMA Treatment of Lymphocytes. Lymphocytes were suspended at 1′ 10⁷ cells/ml in cell culture medium and incubated 1 hour at 37° C. with or without 10 ng/ml PMA (Gibco-BRL). Cells were then washed twice in PBS and used in either the lymphocyte binding assay or analyzed for surface antigens by flow cytometry (see below).

Enzyme Treatment of KG1a or LN. Cytocentrifuge preparations of KG1a or LN frozen sections were glutaraldehyde-fixed, then treated with various enzymes prior to the binding assay. For treatment with neuraminidase (sialidase), slides were rinsed twice with enzyme buffer (50 mM NaAc, 154 mM NaCl, 9 mM CaCl₂, pH 5.5), then incubated 30 minutes at 37° C. with 50 ml of buffer (control) or undiluted neuraminidase (1.2 U/ml, Boehringer Mannheim, Indianapolis, Ind.). In protease studies, slides were incubated with RPMI 1640 alone or RPMI 1640 containing enzymes: 100 U/ml chymotrypsin (Sigma) (115 minutes at 37° C.), or 0.1% bromelain (Sigma) (30 minutes at 37° C.); to assess specificity, the protease inhibitors PMSF (1.0 mg/ml, Sigma) and chymostatin (900 mg/ml, Boehringer Mannheim) were coincubated with chymotrypsin (100 U/ml) for 15 minutes at 37° C. Following enzyme treatments, slides were washed three times with RPMI 1640 and placed in RPMI 1640 with 1 % FBS until use in the binding assay.

KG1a cells in suspension (4′ 10⁷ cells/ml) were incubated with O-sialoglycoprotein endopeptidase (Accurate Chemical and Scientific Corp., Westbury, N.Y.) (0.24 mg/ml, 37° C., 30 minutes), washed three times with 2% FBS in PBS, and cytocentrifuge preparations were made for use in the binding assay. To verify the activity of the enzyme, cells were tested for the cleavage of CD34 by flow cytometry using QBEND10 mAb.

Antigen Expression by Flow Cytometry. Flow cytometric analysis was performed using the following commercially-available mAbs together with isotype-matched controls: TQ1 (anti-L-selectin), LAM1-3 (Anti-L-selectin), 4B4 (anti-VLA-4) (all from Coulter Corp.); QBEND10 (anti-CD34) (AMAC, Westbrook, Me.); anti-CD44, LFA-1-b (anti-CD18), LFA-1-a (anti-CD11a), HPCAS-2 (anti-CD34), anti-CD45, Leukosialin (anti-CD43), anti-Sialyl-Le^{x} (all from Becton Dickinson). Cells (1′ 10⁶) in 100 ml of PBS with 2% FBS were incubated on ice for 25 minutes with antibody as per manufacturer's recommendations, washed ′3 and analyzed on a FACStar^{PLUS} (Becton Dickinson).

Fluorescence Activated Cell Sorting of KG1a cells. KG1a cells were stained with anti-CD34 mAbs (QBEND10-FITC in two experiments, HPCA-2-PE in one experiment) and positive and negative expressing cells were sorted on a FACstar^{PLUS} flow cytometer equipped with an argon laser tuned at 488 nm (Becton Dickinson). Sorted cell populations were restained with anti-CD34 antibody directed at epitopes not used for sorting and were analyzed to determine the efficiency of the sort. Cytocentrifuge preparations were made of the positive and negative sorted fractions and were used in the lymphocyte binding assay.

Transfection of COS-7 with CD34 cDNA. COS-7 cells were transiently transfected with human full-length CD34 cDNA in pCDM8 plasmid (a gift from Dr. Daniel Tenen, Boston, Mass.) using a DEAE Dextran transfection method (Selden, 1992). Briefly, COS-7 cells were incubated for 4 hours at 37° C. with 10 ml of transfection solution containing 20-40 mg of plasmid DNA, 10% Nu Serum (Collaborative Biomedical Products, Bedford, Mass.), 400 mg/ml DEAE Dextran (Sigma), and 100 m chloroquine (Sigma) in Dulbecco's Modified Eagles Medium (Gibcon-BRL). Cells were then rinsed and treated with 10% DMSO (Sigma) in PBS for two minutes at room temperature, rinsed in PBS, and incubated in tissue culture media for three days. In one set of experiments, trypsinization was avoided by growing transfected cells directly on glass slides for subsequent use in the binding assay or for analysis of CD34 expression by fluorescence microscopy. In other experiments, COS-7 cells grown on 10 cm plates were removed with trypsin/EDTA (0.25%/1 mM, Gibco-BRL), then analyzed for CD34 expression by flow cytometry. These trypsinized cells were then placed on slides by cytocentrifuge for use in the lymphocyte binding assay.

Flow Cytometric Analysis of KG la and Immunoprecipitation of ³⁵S-metabolically radiolabeled KG1a. Indirect immunofluorescence staining was performed by incubating 1′ 10⁶ KGla with 1 mg MECA-79 or IgM control antibody (both gifts from Dr. Phillip Streeter, Searle Research Laboratories/Monsanto Co., St. Louis, Mo.) in 2% FBS in PBS for 30 minutes on ice. After washing, the cells were treated with 5 mg FITC-conjugated secondary antibody for 30 minutes on ice. Flow cytometric analysis was performed on a Facscan flow cytometer (Becton-Dickinson). KG1a were metabolically radiolabeled by incubating 100′ 10⁶ cells (20′ 10⁶ cells/ml) for five hours in CRCM30 sulfate-free medium (Sigma Chemical Co., St. Louis, Mo.) supplemented with 150 mCi/ml ³⁵S—Na₂SO₄(Dupont/New England Nuclear, Boston, Mass.). Mesenteric lymph nodes from three mice (˜60 mg of tissue) were minced and radiolabeled in parallel with the KG1a for five hours in CRCM30 sulfate-free medium supplemented with 1 mCi/ml ³⁵S—Na₂SO₄. KG1a and murine lymph nodes were lysed in 2% Triton X-100 in Tris-buffered saline (TBS) containing 1 mM PMSF and 1 mg/ml each of aprotinin, leupeptin and pepstatin A overnight at 4° C. The lysates were clarified by centrifugation at 10,000 g for fifteen minutes and the samples precleared with protein G-agarose for four hours. Immunoprecipitation was performed using equivalent amounts of TCA-precipitable counts from lymph node and KG1a lysates, using 2 mg IgM control antibody or MECA79 followed by 6 mg goat anti-rat IgM secondary antibody (Jackson ImmunoResearch Labs Inc.), or 1 mg anti-CD43 or Ig₁, followed by protein G agarose overnight incubation at 4° C. Samples were electrophoresed (7.5% SDS-PAGE) under reduced conditions followed by autoradiography as described previously (Sackstein et al., 1995).

Chlorate Inhibition of Sulfation. The KG1a adherence assay was performed and quantitated as described previously (Oxley and Sackstein, 1994b). The anti-CD43 antibody L60 (Becton-Dickinson) recognizes a sialylated epitope, thus allowing an independent assessment of the efficiency of neuraminidase treatment of KG1a. KG1a were treated with 0.1 U/ml neuraminidase (V. cholerae, Boehringer Mannheim, Indianapolis, Ind.) for one hour in RPMI 1640 without bicarbonate (Gibco/BRL, Gaithersburg, MD) for sixty minutes at 37° C. Control cells were incubated in RPMI 1640 without bicarbonate with an equivalent volume of neuraminidase storage buffer. The cells were washed in RPMI 1640 twice, tested by the adherence assay to verify complete loss ligand activity, and by flow cytometry to verify loss of the L60 epitope. The neuraminidase-treated KG1a were recultured in RPMI 1640 containing 10% FBS (2′ 10⁶ cells/ml) in the presence or absence of 10 mM sodium chlorate (Sigma Chemical Co.) for twenty-four hours. To verify chlorate inhibition of sulfation, CD43 was immunoprecipitated from equivalent numbers of untreated and chlorate-treated KG1a that were ³⁵S—Na₂SO₄ radiolabeled for the last six hours of the twenty-four hour chlorate treatment.

### Example 1

The procedure for the in vitro binding of human or rat lymphocytes to KG1a cells was adapted from the rat lymphocyte—lymph node (frozen section) binding assay which has been described by Stamper and Woodruff (1976) and Sackstein et al. (1988) as described herein above. Cytocentrifuge preparations of KG1a or other cell lines were made on a Cytospin 3 Cytocentrifuge (Shandon Lipshaw, Pittsburgh, Pa.) following the manufacturer's instruction (Cytospin®3 Cell Preparation System Operator Guide) with modifications of the sample chamber to provide appropriate placement of the cytocentrifuge cell pellet onto slides for use in an adherence assay as described herein above.

Cell Separation: Cell separation procedures for blood cells as are generally known to those skilled in the art are used to separate lymphocytes and granulocytes from peripheral blood samples, from bone marrow aspirates, and from vertebral bodies donated through organ harvests. More specifically:

Peripheral Blood

(1) Sodium citrated blue top tubes are used, spun approximately 30 seconds at 100 g to pull all cells away from the top of the tubes. The blood is removed, 25 ml or less, and placed into a 50 ml Falcon® tube. Using sterile 1M PBS, add equal volume to the volume of blood for a 1:1 final dilution.

(2) Place 10 ml of FICOLL lymphocyte separation medium into a fresh 50 ml tube. Layer the blood from step 1 onto the top of the FICOLL medium slowly and carefully to insure the interface is tight. Place no more than 35 ml of diluted blood on top of the FICOLL layer so that the total volume inside the tube does not exceed 45 ml. Spin at 400 g for 30 minutes.

(3) Remove the upper clear layer containing the plasma and platelets; remove the next layer (cloudy, lymphocyte layer, PBL: peripheral blood lymphocytes), and place in a 50 ml tube. Suspend the PBL to 45 ml in 1′ PBS. Spin them again for 10 minutes at slightly less than 200 g (any more force than this will cause platelet contamination). Count cells, wash and spin at 1000 rpm twice and resuspend at 3 to 4 million cells per ml which is the volume necessary to achieve correct cell density in the cytocentrifuge procedure.

(4) Remove the clear FICOLL layer from the tubes from step (3) and discard leaving only the granulocytes and monocytes in the red cell pellet. Dilute the pellet 1:1 by volume with 1′ Hank's Balanced Salt Solution (HBSS). Dilute this volume 1:1 with 3% Dextran (made by dissolving 1.5 g of Dextran powder into 50 ml of sterile 1′ PBS) and shake gently to mix. Allow to stand for 25 minutes so that the red blood cells pellet out under gravity, leaving the granulocytes and monocytes in the supernatant. After 25 minutes, remove the supernatant and dilute it to 45 ml in 1′ PBS. Discard the red cell pellet. Spin the granulocytes and monocytes again for 10 minutes at 200 g. To remove remaining red cells treat pellet with a salt gradient. Count the cells and wash one more time at 200 g for 10 minutes before resuspending to 3 to 4 million cells per ml. Both groups of cells (PBL and granulocytes) can be held on ice until ready for cytocentrifuge.

Bone Marrow Aspirates: Bone marrow aspirates should be requested in liquid-heparinized tubes. The tube should be well shaken to make sure any cells adhering to bone and tissue are released into the liquid portion of the sample. The tube should then be placed into a 15 ml Falcon® tube and spun for 10 minutes at 1200 rpm. Remove the top layer of bone and tissue from-the surface and discard. The remaining aspirate is diluted in 1′ PBS and treated as if it were a peripheral blood sample in the above procedure.

Though the aspirate material is not supposed to contain red blood cells, FICOLL preparation is still useful for removing bone and tissue which was not removed in the first wash. The cells removed from above the FICOLL layer should be labeled “bone marrow cells” and should be prepared for cytospin in the same manner and to the same concentration as were the PBLSs.

Vertebral Bodies: When working with the vertebral bodies, it is extremely important to remember the danger associated with working with patient blood. Due to the nature of the procedure used to retrieve the desired cell populations, special precautions must be taken to prevent contact with the fluids and tissues of the sample by the researcher. A full frontal covering must be worn along with gloves and a face guard.

(1) Cut the vertebral body in half and remove the marrow and placed into a solution of heparin. It is better to remove many very small pieces of marrow instead of just a few large chunks. This allows for more surface area of marrow to be exposed, and therefore, more cells can be collected from the bone marrow matrix.

(2) Once all of the marrow has been removed, shake the tube containing the heparinized marrow vigorously, in order to free any cells attached to the matrix. Let the tube stand for a few seconds to allow all of the dense marrow material to sink to the bottom of the tube. Remove the supernatant solution and place it into a clean tube. Resuspend the chunks of marrow in heparin again and shake vigorously to free more cells. Again, remove the supernatant. Repeat several times. Once the marrow is cleansed of most of the adherent cells, spin the supernatant materials in the centrifuge for 10 minutes at 200 g. Resuspend the pellet in RPMI medium and pass the suspension through a sterile nylon mesh filter into a clean 50 ml tube. This will remove any remaining marrow pieces. The remaining cells should be counted and washed two more times before being suspended to proper concentration (3 to 4 million per ml) in RPMI medium for cytocentrifuge.

After each of the cell populations has been separated, washed, and suspended to proper concentration, the following procedure is used to place the cells onto slides in order to test the binding capacity of each population. The cytocentrifuge will pull cells out of small volumes of solution and place them one cell layer thick onto slides. If the concentration of cells in solution is too high, cells will be stacked on top of one another and the experiment will not produce useful results. If the concentration is too low, the actual numerical data collected from the experiment will not be statistically valuable. Cell suspensions should have a concentration of only 3 to 4 million cells.

Cytocentrifuge Procedure

(1) As described herein above, place a cell suspension on a slide and place on the rotational table under the experimental conditions of the adherence assay. Observe where cells aggregate and prepare a model slide (FIG. 17) showing this location by which to mark all other slides.

(2) After marking the proper number of slides (i.e. typically one slide/2.5′ 10⁵ cells), take the same number of clamps, funnels, and filter cards (paper guides) (FIG. 10) as slides needed. Assemble the holder assembly and load in cytocentrifuge as per the instructions in the cytocentrifuge manual. Once the holder assemblies are loaded, place 100 ml of the proper cell suspension in each funnel. Set speed at 570 and time 5 minutes for this particular assay. As per instruction in manual remove slides from holding member after spin. Do not smear or wipe the delicately placed cells from the slide. Allow slides to dry for one minute.

(3) When dry, place the slides into a 3% glutaraldehyde solution (3.6 ml of 25% glutaraldehyde to 26.4 ml of 1′ PBS per 12 slides) for ten minutes. After the 10 minutes of fixing, rinse the slides twice, gently, in 1′ PBS. Place the slides into lysine solution (0.2M) for 10 minutes. This solution will prevent nonspecific binding in the adherence assay. After lysine treatment, rinse slides twice in RPMI and then place them into 1% FBS in RPMI at 4° C. until ready to perform the adherence assay.

### Example 2

Lymphocytes Bind to KG1a. Lymphocytes (both PBL and TDL) adhered specifically and reproducibly to KG1a, but not to RPMI 8402, HL60, Nalm 16, K562, or Raji cell lines in the in vitro binding assay (Table 1). All experiments were performed in parallel with LN frozen sections as positive controls. Lymphocyte binding to KG1a was observed under conditions identical to those whereby L-selectin mediates binding of lymphocytes to LN HEV.

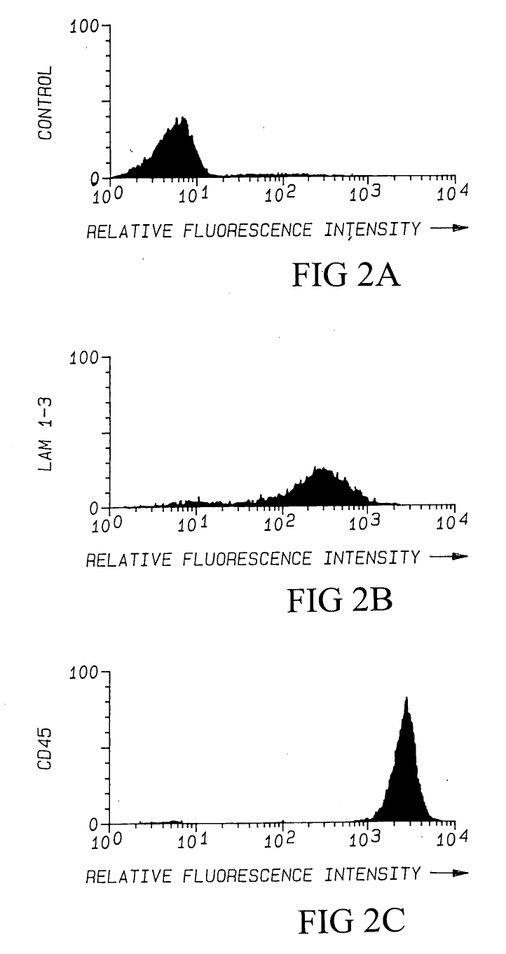

Lymphocyte Binding to KG1a is Mediated by L-selectin. To directly examine whether lymphocyte attachment was mediated by L-selectin, PBL were pre-incubated with the anti-L-selectin mAb LAM1-3, anti-CD45, or IgG₁ isotype control antibodies. The LAM1-3 antibody completely inhibited lymphocyte binding to KG1a and LN control, while CD45 and isotype control mAbs did not affect binding (FIG. 1A & 1B). In order to quantify the relative amounts of antibody attachment to lymphocytes, antibody-treated lymphocytes were incubated with goat-anti-mouse FITC-conjugated secondary antibody and analyzed by flow cytometry. Although the amount of anti-CD45 antibody on lymphocytes was significantly greater than that of LAM1-3 as indicated by mean channel fluorescence (FIG. 2A-C), LAM1-3 alone blocked lymphocyte adherence to KG1a and LN HEV, indicating that this effect was specific and not secondary to charge or stearic alterations of the lymphocyte membrane.

The Effect of Enzyme Treatment of KG1a on Lymphocyte Binding. Pretreatment of both KG la and LN control sections with neuraminidase (60 mU), chymotrypsin (100 U/ml) or bromelain (0.1%) prior to the binding assay abrogated binding of lymphocytes, while treatment with buffer or medium alone did not alter binding capacity. In addition, the effects of chymotrypsin were confirmed by coincubation with the protease inhibitors chymostatin and PMSF, which prevented chymotrypsin effects on lymphocyte binding. However, pretreatment of KG1a with O-sialoglycoprotein endopeptidase had no effect on lymphocyte binding despite complete enzymatic removal of the CD34 epitope recognized by QBEND10 mAb (see Table 2).

Lymphocyte Binding to KG1a is Calcium Dependent. Lymphocyte binding to KG1a and to LN control sections was completely inhibited by the presence of EDTA, indicating a calcium requirement for lymphocyte-KG1a binding.

Mannose-6-Phosphate, PPME, and Fucoidin Inhibit Lymphocyte Binding to KG1a. The specificity of lymphocyte-KG1a binding was investigated by treating PBL or TDL with carbohydrate inhibitors of L-selectin-HEV interactions prior to the adherence assay. Man-6-P (10 mM), PPME (10 mg/ml), and fucoidin (5 mg/ml) all inhibited lymphocyte binding to both KG1a and LN control sections (see Table 2).

PMA Treatment of Lymphocytes Results in the Loss of Binding to KG1a. PBL were incubated for 1 hour at 37° C. with 10 ng/ml PMA, then used in the lymphocyte binding assay. PMA-treated PBL were unable to bind to either KG1a or LN HEV, while control PBL demonstrated high amounts of binding (see Table 2).

Loss of surface L-selectin was assessed by flow cytometric analysis of TQ1 levels in control and PMA-treated PBL. PMA-treated lymphocytes showed a dramatic decrease in TQ1 mean channel fluorescence (to levels less than 10% of that of untreated cells) in three separate experiments. PMA-treated PBL were also analyzed for expression of CD44, LFA-1 (both a and b chains), and VLA-4, and expression of these adhesion molecules following PMA exposure was identical to expression on control PBL.

### Example 3

Pretreatment of KG1a with Anti-CD34 Antibodies Did Not Inhibit Adherence of Lymphocytes. Cytocentrifuge preparations of KG1a were preincubated with anti-CD34 antibodies and the binding assay was performed in the presence of the antibodies (Table 2). Monoclonal antibodies to four different CD34 epitopes were used alone or in combination, including the clones My10, QBEND10, 8g12, and 12.8, in amounts ranging from 0.2 to 17 mg/slide. Anti-CD45 (irrelevant control) and IgG₁ (isotype control) antibodies were also tested. None of the anti-CD34 antibodies inhibited lymphocyte binding to KG1a, despite immunohistochemical evidence of extensive antibody binding to the glutaraldehyde-fixed KG1a sections.

Other Surface Antigens on KG1a do not Appear to Mediate Binding. The surface expression of several antigens on KG1a, RPMI 8402, HL60, Nalm 16, K562, and Raji was analyzed by flow cytometry (Table 1). LFA-1, FLA-4, CD44, Sialyl Le^{x}, and CD43 were all expressed by KG1a and at least one other cell line that did not support lymphocyte adherence. Of note, although RPMI 8402 cells express CD34 at levels comparable to KG1a, there was no adherence of lymphocytes to these cells in the binding assay.

CD34 Positive and Negative KG1a Cells Supported Equivalent Amounts of Lymphocyte Binding. CD34+ and CD34− KG1a cells were separated by fluorescence activated cell sorting and cytocentrifuge preparations of each population were made. The in vitro adherence of lymphocytes was identical in the CD34+ and CD34− populations despite an enrichment of >90% and <10% CD34+ cells in the respective populations (FIGS. 3A-D and 4; Table 2).

### Example 4

CD34-Transfected COS-7 Cells Did Not Support Lymphocyte Adherence. COS-7 cells were transfected with CD34 and tested in the in vitro binding assay, and both trypsinized and intact transfected COS-7 cells failed to support lymphocyte adherence. By flow cytometric analysis, transfected cells were approximately 60% positive for CD34 expression, and the mean channel fluorescence was greater than that of KG1a control cells (FIG. 5A-F). Intact, untrypsinized COS-7 cells transfected with CD34 also strongly expressed CD34 (>>90% positive as estimated by fluorescence microscopy).

### Example 5

Flow cytometric analysis of KG1a and immunoprecipitation of ³⁵S-metabolically radiolabeled KG1a demonstrate an absence of MECA 79 antigen.

In functional studies, incubation of KG1a cytocentrifuge layers with MECA 79 or isotype-control (IgM) rat monoclonal did not inhibit lymphocyte attachment in the adherence assay (e.g., for studies using MECA 79 at a concentration of 50 mg/ml, binding was 98.2%±3.38% (mean±SEM) that of control assays without antibody addition). However, as is characteristic of MECA 79, lymphocyte attachment to murine lymph node HEV was blocked using MECA 79 at concentrations as low as 2 mg/ml. To investigate surface expression of MECA 79 antigen on KG1a cells, flow cytometric analysis of MECA-79 antigen was performed on KG1a cells and compared with isotype matched control antibody (FIGS. 6A and 6B). Although lymph node HEV were clearly and characteristically identified by staining with MECA 79 as evident by fluorescence microscopy. There was no MECA 79 staining evident by flow cytometric analysis of KG1a cells by fluorescence microscopy.

To further evaluate for expression of MECA 79 antigens by KG1a, the cells were incubated with either ³⁵S—Na₂SO₄ or ³⁵S-methionine/cysteine and resultant radiolabeled protein was immunoprecipitated using MECA 79 antibody (FIG. 6C). As a positive control, murine lymph nodes were radiolabeled, lysed, and immunoprecipitated in parallel with KG1a. To adjust for potential differences in efficiency of radioactive label incorporation between KG1a and murine lymph nodes, the relative amounts of lysate immunoprecipitated from each source were equalized for TCA-precipitable counts. A representative immunoprecipitation of MECA 79 from ³⁵S—Na₂SO₄-labeled KGLa and lymph node is shown in FIG. 6C. The characteristic 50,000 and 90,000 mw bands are immunoprecipitated by MECA 79 in lymph node lysate but no MECA 79 bands are detected in the KGla lysate; similar results were obtained with MECA 79 immunoprecipitation of ³⁵S-methionine/cysteine labeled KG1a lysates. In the figure, the faint band at ˜90,000 mw shown on both MECA 79 and rat IgM-control antibody immunoprecipitates of KG1a cells is non-specific as it can be removed by extensive preclearing of lysates with protein-G agarose alone; a similar faint 90,000 mw band could also be visualized on IgG1 control immunoprecipitation of ³⁵SO₄-labeled KG1a cells. Of note, the sulfated glycoprotein CD43 (˜110 kDa) was readily immunoprecipitated from KG1a lysates, verifying that ³⁵SO₄ was incorporated into newly synthesized products and that immunoprecipitation conditions were appropriate for detection of specific radiolabeled KG1a proteins.

### Example 6

Chlorate inhibition of sulfation does not block lymphocyte binding to KG1a. As shown herein above, the KG1a L-selectin ligand, like the endothelial ligands, requires sialylation for function, as treatment of KG1a with neuraminidase completely abolishes L-selectin-mediated lymphocyte binding. Applicant, therefore, measured the kinetics of recovery of ligand activity following neuraminidase treatment, and chose experimental conditions such to maintain chlorate inhibition of sulfation throughout the period when the KG1a ligand is being re-expressed on the cell membrane. Cell viability following neuraminidase digestion was typically >99% by trypan exclusion.

Initial studies of the kinetics of recovery of binding activity following neuraminidase treatment showed absence of ligand activity for 10-12 hours; thereafter, ligand activity increased steadily with return to baseline levels within 24 hours. Return of binding activity following desialylation was blocked by metabolic inhibition of N-linked glycosylation by tunicamycin and by inhibition of protein synthesis by cycloheximide (Table 3). Of interest, the results of tunicamycin and cycloheximide experiments indicate that the membrane turnover of the L-selectin ligand is relatively slow in resting cells, as culture of KG1a in the presence of these reagents for up to 20 hours did not alter L-selectin ligand activity (Table 3). However, following neuraminidase treatment of KG1a, both reagents inhibited return of ligand activity without affecting cell viability (trypan blue exclusion >98%), suggesting that re-expression of ligand activity results from de novo synthesis and post-translational processing of ligand and not from transport of pre-formed ligand from intracellular compartments to the membrane surface.

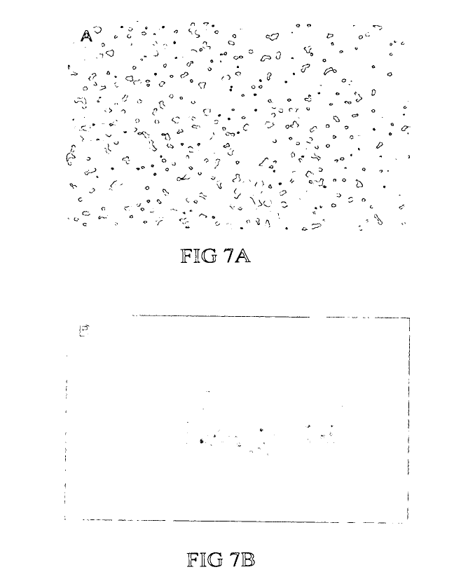

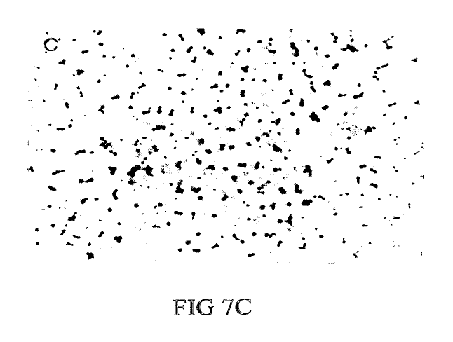

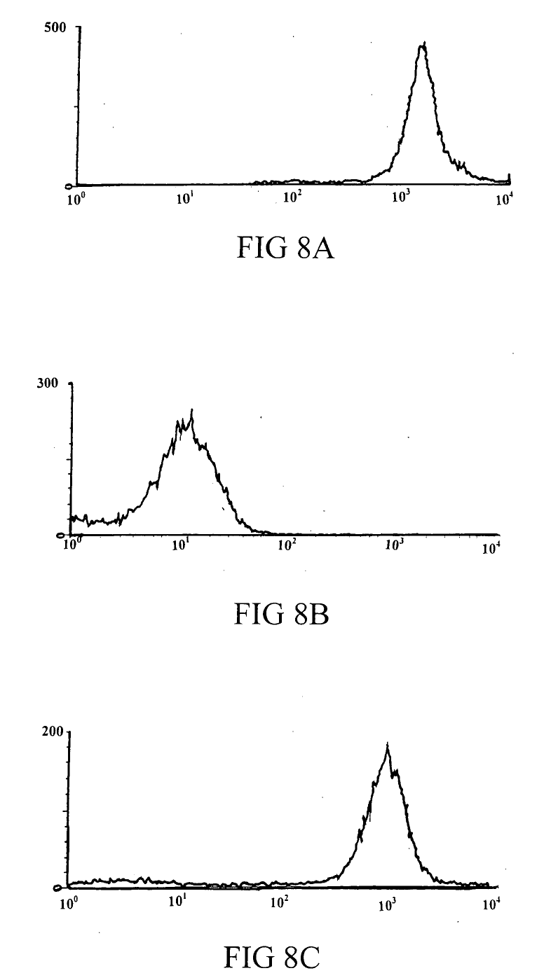

The cleavage of sensitive sialic acid epitopes following neuraminidase treatment of KG1a was confirmed by testing for loss of ligand activity in the adherence assay and by measuring the expression of the sialic acid-dependent L60 epitope of CD43 by flow cytometry (Table 3; FIGS. 7 and 8). L60-negative/ligand activity-negative neuraminidase-treated KG1a were incubated without chlorate (control) or in presence of chlorate for 24 hours to inhibit sulfation throughout the duration of ligand reexpression. Cell viability was >95% in both chlorate-treated and control cell populations (trypan blue exclusion). Twenty-four hours of chlorate treatment (FIG. 7C) did not alter the re-expression of ligand activity; indeed, quantitation of adherence assays performed on KG1a 24 hours after neuraminidase treatment revealed that L-selectin ligand activity in chlorate-incubated KG1a cells was slightly higher than that of non-incubated control cells (Table 3). Quantitated lymphocyte adherence to chlorate-incubated KG1a cells was 107%±6% (mean±SEM) that of neuraminidase-treated KG1a cells not incubated in chlorate. As with control KG1a cells, lymphocyte adherence to sulfate-treated KG1a was L-selectin-specific: it was Ca++-dependent, was completely inhibited by anti-L-selectin monoclonal antibodies known to block L-selectin adhesive function and by carbohydrate molecules known to bind L-selectin (e.g., fucoidin), and eliminated by phorbol myristate acetate (PMA) treatment of lymphocytes to induce L-selectin shedding.

Sequential ³⁵S—SO₄-pulse radiolabelling studies at eight hour time intervals -within the 24 hours of chlorate incubation indicated that sulfation was inhibited throughout the entire incubation period, as demonstrated by diminished quantities of TCA-precipitable radiolabeled protein counts (chlorate-incubated counts consistently <10% that of non-chlorate treated controls) and of total ³⁵SO₄-radiolabelled proteins observed by SDS-PAGE/autoradiography of cell lysates (FIG. 9A,B). However, chlorate did not inhibit total protein synthesis as ³⁵S-methionine/cysteine incorporated TCA-precipitable counts were not significantly different in chlorate and control groups, ³⁵S—SO₄-radiolabelling of CD43 was markedly diminished by chlorate treatment, even in the terminal eight hours of the 24 hour incubation. Similar to the results of recovery of ligand activity, chlorate treatment did not block re-expression of CD43 to baseline levels (FIG. 8C) despite evident sulfation-deficiency (FIG. 9). Monitoring of membrane CD43 recovery was performed using a sialylated epitope of the protein, indicating that sialylation of CD43 (like sialylation of the L-selectin ligand) was not affected by chlorate treatment.

Flow cytometric analysis of CD43 shown in FIG. 8 demonstrated a loss of a sialylation-specific epitope following neuraminidase treatment (FIG. 8B, bottom) relative to control levels (FIG. 8A, top).

Inhibition of sulfation by sodium chlorate is shown in FIG. 9 demonstrated by immunoprecipitation/SDS-PAGE analysis of ³⁵S—SO₄ incorporation into CD43. Numbers at the left of the figure are molecular weights in kDa.

### Example 7

### PSGL-1 Analysis

P-selectin glycoprotein ligand-1 (PSGL-1) is a cell surface mucin-like glycoprotein which serves as a ligand for both P- and E-selectin. This protein is expressed on a variety of myeloid cell lines, including HL60 cells. In order to determine whether the L-selectin ligand activity of KG1a is related to PSGL-1, Applicants performed flow cytometric analysis of PSGL-1 expression on KG1a, blocking studies utilizing both a P-selectin-Ig chimera and an anti-PSGL-1 monoclonal antibody, and enzymatic cleavage and metabolic studies of PSGL-1 and L-selectin ligand activity. These studies reveal that KG1a cells express immunoreactive and functional PSGL-1, however, expression of PSGL-1 is separable from L-selectin ligand activity: (1) blocking of PSGL-1 function using P-selectin-Ig chimera and anti-PSGL-1 monoclonal antibody had no effect on ligand activity of KG1a cells; (2) cleavage with the proteases mocarhagin and O-sialoglycoprotease each abrogated PSGL-1 recognition by P-selectin-Ig chimera and L-selectin activity of PSGL-1, but had no effect on L-selectin ligand activity; (3) metabolic inhibition of sulfation by chlorate eliminated P-selectin ligand activity of PSGL-1 but did not alter L-selectin ligand activity; and (4) KGla sorted for high-level (>95%) and low-level (>5%) expression of PSGL-1 had equivalently high L-selectin ligand activity, and, moreover, HL60 cells were completely devoid of L-selectin ligand activity despite high PSGL-1 expression. These studies indicate that although PSGL-1 is capable of serving as a ligand for E- and P-selectin, it does not itself, display L-selectin ligand activity. The independent relationship between PSGL-1 and L-selectin ligand activity indicates these functions are subserved by distinct molecules.

### Example 8

### Methods

Hematopoietic Cell Lines and Isolation of Leukemia Cells from Patients. All hematopoietic cell lines were propagated in RPMI1640/10%FBS/1% penicillin-streptomycin (Life Technologies, Inc., Grand Island, N.Y.).

To analyze the L-selectin ligand activity of CD44 from AMLs, leukemic cells were isolated by Ficoll-Hypaque (1.077-1.0800 g/ml) (ICN Biomedicals, Inc., Aurora, Ohio) density gradient centrifugation of whole blood from patients with leukemias; leukemia blasts represented >80% of all circulating leukocytes. Cells were cultured short-term and maintained in RPMI1640/20%FBS/1%penicillin-streptomycin.

Antibodies, Enzymes and Metabolic Inhibitors. Anti-CD44 mouse monoclonal antibody, A3D8, was purchased from Sigma-Aldrich Chemical Co., St. Louis, Mo. Rat IgG monoclonal Abs against CD44 (Hermes-1 and IM7) were obtained from Dr. Brenda Sandmaier of the Fred Hutchinson Cancer Research Center. Mouse IgG anti-CD34 monoclonal Ab QBEND-10 was purchased from Coulter-Immunotech, Miami, Fla. Rat IgM monoclonal Ab, HECA-452 was purchased from Pharmingen, San Diego, Calif. All fluorochrome- and alkaline phosphatase (AP)-conjugated secondary Abs and isotype controls were obtained from Zymed, San Francisco, Calif.

O-sialoglycoprotein endopeptidase (OSGE) was purchased from Accurate Chemicals, Westbury, N.Y. Vibrio cholera neuraminidase, which digests α2,3, α2,6 or α2,8-linked sialic acids, and N-glycosidase F (PNG-F), which cleaves all types of N-glycans, were purchased from Roche Molecular Biochemicals, Indianapolis, Ind.

Sodium chlorate, a metabolic inhibitor of sulfation, and tunicamycin, a metabolic inhibitor of N-glycosylation, were purchased from Sigma.

Radiolabeling of KG1a cellular proteins. KG1a cultures were incubated with 200 μCi/ml 2-[3H]-D-Mannose (NEN Life Science Products, Boston Mass.) for six hours in glucose-free DMEM growth medium (Gibco), and membrane preparations were prepared as described below. KG1a cells were also labeled for eight hours with [³⁵S]—Na₂SO₄ (150 μCi/ml) and [³⁵S]-EasyTag™-L-Methionine (150 μCi/ml) with or without sodium chlorate (10 mM), and membrane proteins were prepared as discussed below.

Preparation of Cellular Membrane Proteins for SDS-PAGE and Immunoblot Analysis of HECA-452-Reactive Proteins and CD44. Membrane preparations of human leukemia cell lines, KG1a, HL-60, K562, and RPMI-8402, or of leukemia cells from patients were isolated in the following manner. Cell suspensions (20-40×10⁷/ml), in Complete™ protease inhibitor cocktail buffer (Roche Molecular Biochemicals), were placed in a French Pressure Cell in which the pressure was elevated to 400 psi, and cell membranes were harvested by a sudden release to atmospheric pressure. Cell lysates were subjected to differential centrifugation to separate cellular contents and to isolate membrane fractions. Protein concentrations were assessed using Bradford Reagent (Sigma Chemicals Co., St. Louis, Mo.), and membrane preparations were stored in aliquots at −20° C. For SDS-PAGE and Western blotting, membrane preparations (30 μg/sample) were diluted in reducing sample buffer and separated on 6 or 9% SDS-PAGE gels. Resolved membrane proteins were transferred to Sequi-blot™ PVDF membrane (Bio-Rad, Inc., Hercules, Calif.) and, prior to immunoblotting with primary Ab, blocked with PBS/Tween-20/20%FBS from one hour at 4° C. Blots were incubated with HECA-452 (1.2 μg/ml PBS), Hermes-1 (2.0 μg/ml PBS) or A3D8 for one hour at RT. Isotype control immunoblots using either rat IgM, mouse IgG or rat IgG were performed in parallel to rule out non-specific reactive proteins. After washing three times for 15 minutes with PBS/0.1%Tween-20, blots were incubated with either AP-conjugated rabbit anti-rat IgM Abs (1:400), AP-conjugated goat anti-rat IgG (1:200) or AP-conjugated goat anti-mouse IgG (1:8000) depending on the primary Ab. Alkaline phosphatase substrate, Western Blue® (Promega, Madison, Wisc.), was then added to develop the blots, and the reaction was stopped with PBS prior to significant background staining.

To determine whether HECA-452 epitopes on KG1a membrane proteins were dependent on sialic acid and/or O-linked sialoglyoproteins, membrane preparations were pretreated with either neuraminidase (0.1 U/ml for one hour at 37° C.) or O-sialoglycoprotein endopeptidase (60 μg/ml for one hour at 37° C.) prior to SDS-PAGE and Western blotting.

Alternatively, to assess whether HECA-452-reactive epitopes on KG1a membrane proteins were displayed on N-glycans, KG1a cultures were treated for one hour at 37° C. with neuraminidase (0.1 U/ml), washed the cells 2× in PBS, added tunicamycin (15 μg/ml) or DMSO (diluent control) and incubated for 24 hours at 37° C. Since neuraminidase completely abrogates HECA-452 reactivity, pretreating KG1a cells in this manner allowed for the direct assessment of de novo synthesized sialylated N-glycosylated proteins in the presence of an N-glycosylation inhibitor.

Sequential Purification of HECA-452-Reactive KG1a Proteins by Differential SDS-PAGE. Since other proteins from crude KG1a membrane preparations could have co-migrated with HECA-452-reactive proteins resulting in a false positive identification of a candidate protein, a 3-step scheme was devised for the isolation of a semi-pure KG1a protein species whose gel mobility was changed by reduction conditions and percentage of acrylamide. Step one involves KG1a membrane preparations (50 μg lane), the membrane preparations were first resolved on a nonreducing 6% SDS-PAGE gel. A gel run in parallel was prepared, and proteins were transferred to PVDF membrane and blotted with HECA-452. This HECA-452 immunoblot was placed under the gel and acted as a guide for the excision of the relevant 95 kDa band (1st Step). In step two, this gel fragment was placed into a reducing 9% gel (2-β-mercaptoethanol was added to the well) prior to running. Again, a gel was run in parallel, transferred to PVDF membrane and immunoblotted with HECA-452 and the 98 kDa band was excised. Finally, in step 3, this gel fragment was placed into another 9% gel, and the process was repeated. Following the third step, the gel was stained (Coomassie-blue), and submitted for identification by mass spectrometry.

Immunoprecipitation of CD44. Membrane proteins of hematopoietic cell lines or of leukemia cells from patients prepared as described above were solubilized in 2% Nonidet P-40 and precleared in Protein G-agarose (Gibco). Protein concentrations were also requantified as described above. Samples (100 μg) were incubated with anti-CD44 monoclonal Ab Hermes-1 (3 μg) or rat IgG (3 μg) isotype control for four hours at 4° C. The antibody-lysate mixture was added to 30 μl of Protein G-Agarose and incubated for one hour at 4° C. under constant rotation. Immunoprecipitates were washed three times with lysis buffer/2%NP-40/1%BSA, diluted in reducing sample buffer, boiled for five minutes at 100° C., subjected to 6% or 9% SDS-PAGE, transferred to PVDF membrane and immunoblotted with monoclonal Abs HECA-452, A3D8 or Hermes-1 as described above. When immunoprecipitated CD44 was prepared for analysis in the Stamper-Woodruff adherence assay²¹, immunoprecipitates were diluted in lysis buffer/2%NP-40, boiled for five minutes at 100° C., cooled on ice and spotted onto glass slides.

Hydrodynamic Flow Analysis of L-selectin Ligands on Blotting Membrane. Using a parallel-plate flow chamber, L-selectin-mediated adhesive interactions between KG1a membrane proteins immobilized on PVDF blotting membrane and rat thoracic duct lymphocytes expressing high levels of L-selectin under defined shear force conditions were studied. L-selectin-expressing lymphocytes were isolated as previously described^{18,19}, washed twice in HBSS and suspended at 2×10⁷/ml in HBSS/10 mM HEPES/2 mMCaCl₂ (H/H/Ca⁺⁺)/10% glycerol. Western blots of KG1a membrane preparations stained with HECA-452, A3D8 or Hermes-1 were rendered transparent by incubating them in H/H/Ca⁺⁺/10% glycerol. These blots were then placed under the parallel plate flow chamber, and lymphocytes were perfused into the chamber. After allowing the cells to come in contact with the blotting membrane, the flow rate was adjusted accordingly to exert a shear force of 2.3 dynes/cm² to study L-selectin-mediated adhesive interactions. The viscosity of 10% glycerol adhesion assay medium was considered in the calculation of shear force values, which were increased by 30% compared with shear forces originally calculated for H/H/Ca⁺⁺ medium without glycerol²³. The number of lymphocytes rolling on and between each immunostained banding region was quantified from five independent fields under 200× magnification on the video monitor using molecular weight markers (Kaleidoscope Molecular Weight Markers, Bio-Rad Lab.; See Blue® from Novex, Inc.) as guides to help align and visualize the apparent molecular weights of the proteins of interest. A minimum of three experiments were performed and results were expressed as the mean of cell rolling/field. Negative controls were prepared by either adding 5 mM EDTA to the lymphocyte H/H assay buffer to chelate Ca⁺⁺ required for binding, by using lymphocytes treated with PMA (50 ng/ml, which induces the cleavage of L-selectin), or by treating the lymphocytes with anti-rat L-selectin Abs (10 μg/ml; HRL-1) (Pharmingen) to verify the sole contribution of L-selectin.

Mass Spectrometry of Proteins Separated by SDS-PAGE. To identify KG1a membrane proteins exhibiting L-selectin ligand activity, gel fragments from the 3-step SDS-PAGE purification schema were submitted, which led to the exact region on the Western blot that is stained by HECA-452 and supported ligand activity. Matrix-Assisted Laser Desorption Ionization-Time of Flight (MALDI-TOF) mass spectrometry of trypsin-digested gel fragments was performed by the Molecular Biology Core Facility at Dana Farber Cancer Institute. Using the UCSF Mass Spectrometry Facility MS-Fit Program, the NCBlnr database was searched for possible peptide matches with the mass spectrometry data generated from trypsin digests.

### Example 9

Though several L-selectin ligands have been identified, the understanding of the structure, function and overall breadth of L-selectin ligands is incomplete. For all naturally expressed glycoprotein L-selectin ligands described to date, the binding determinants are present on sialylated, fucosylated lactosamines displayed on O-linked carbohydrates (14,15). On high endothelial venule cells from lymph nodes, multiple L-selectin ligands have been described (16-19), all of which are glycoproteins reactive with a monoclonal antibody known as MECA 79 (20). One of these ligands is a glycoform of CD34 (21). Although present on endothelial cells in most tissues, CD34 is an integral membrane glycoprotein best known as a marker of the multilineage colony-forming hematopoietic “stem cell” (22). The CD34 marker was originally identified on the KG1a cell line, the most primitive human hematopoietic progenitor cell line and the one which most closely resembles, phenotypically, the native human stem cell (22-24).

The finding that CD34 as expressed on lymph node endothelium served as an L-selectin ligand led to the examination, in early studies, of whether CD34 on hematopoietic cells functioned similarly. In these studies, a conventional shear-based, L-selectin-specific Stamper-Woodruff assay system (25) was used, whereby lymphocytes expressing L-selectin were overlaid onto cyto-spin preparations of hematopoietic progenitor cells and cell lines, and L-selectin-dependent adherence interactions were analyzed. These investigations not only showed that the CD34 glycoform present on hematopoietic cells does not function as an L-selectin ligand in the Stamper-Woodruff assay, but also revealed that another (non-CD34) L-selectin ligand is expressed in the hematopoietic progenitor cell line KG1a and on normal human bone marrow CD34+ hematopoietic progenitor cells (2,26), providing the first evidence of an L-selectin ligand naturally expressed on a non-endothelial cell type. Like endothelial L-selectin ligands, this hematopoietic cell L-selectin ligand (HCELL) requires sialylation for activity, however, HCELL otherwise differs in several fundamental ways from previously described endothelial L-selectin ligands, it does not contain epitopes recognized by monoclonal antibody MECA 79, it is resistant to digestion with O-sialoglycoprotease (OSGP), a peptidase which cleaves at sites of O-linked sialylated carbohydrate modifications, and its activity is sulfation-independent (27).

Prior to the present description of HCELL activity on hematopoietic progenitors, several groups reported that P-selectin glycoprotein ligand-1 (PSGL-1), a cell surface mucin-like glycoprotein which serves as a P-selectin and E-selectin ligand (28,29), can also serve as an L-selectin ligand (30-33). The location of the L-selectin binding determinant on PSGL-1 appears to be similar to that of the P-selectin binding site as both binding activities are localized to a mocarhagin digestion-sensitive N-terminal portion of the molecule (32). PSGL-1 is expressed on neutrophils and on a variety of human myeloid cell lines, including HL60 and KG1a cells (34-36). Like HCELL, the finding that PSGL-1 is also expressed on normal hematopoietic progenitor cells (36,37) highlights the potential role(s) for selectin-ligand interactions in the biology of hematopoiesis. Indeed, there is evidence that PSGL-1 may function as an inhibitor of hematopoiesis, in that binding of P-selectin to PSGL-1 expressed on human CD34+ hematopoietic progenitors inhibits their proliferation (6).

In contrast to the finding that engagement of PSGL-1 can inhibit hematopoiesis, the observations that L-selectin expression on human hematopoietic progenitors is correlated with higher clonogenic activity and faster engraftment following stem cell transplantation suggests that hematopoietic cells possess L-selectin ligands other than PSGL-1 that can promote the growth and differentiation of primitive progenitors. In this context, a recent study has shown that although all three selectins are capable of mediating discrete adhesive interactions with human adult bone marrow and fetal liver CD34+ hematopoietic progenitor cells under shear conditions (38), binding to L-selectin is most pronounced on more primitive (CD34+/CD38−) cells compared to more mature (CD38+) progenitors. Moreover, antibody blocking studies indicate that progenitor cell interactions with L-selectin are not mediated principally through either PSGL-1 or CD34 (38). These findings suggest there is lineage-specific regulation of expression of novel L-selectin ligands during hematopoietic development, and underscore the need to define and characterize the surface molecules on human hematopoietic cells which serve as L-selectin ligands. Accordingly, to further clarify the structural biology of HCELL and to analyze its relationship to PSGL-1, a variety of chemical treatments were performed, enzymatic digestions and metabolic studies of intact cells and isolated membrane preparations of KG1a cells. The results of these studies indicate that HCELL is distinct from PSGL-1. Furthermore, the data show that HCELL is a membrane-anchored glycoprotein which is structurally distinguished from all previously described native glycoprotein L-selectin ligands in that determinant(s) conferring L-selectin binding activity are presented on N-linked complex carbohydrates.

Materials and Methods

Cells, antibodies and enzymes. Cells: KG1a and HL60 cells, originally obtained from ATCC, were cultured in RPMI 1640 (Gibco-BRL, Gaithersburg, Md.) containing 10% fetal bovine serum (FBS) in a humidified chamber at 37° C. with 5% CO₂.

Antibodies: Anti-PSGL-1 antibody PL-1 (which blocks P- and L-selectin binding to PSGL-1 (30-32, 39)) was obtained from Pharmingen. Functional P-selectin-Ig fusion protein, murine monoclonal antibodies (PSL-275, 2G3, 4F9 and 4D8) and isotype control monoclonals, and rabbit polyclonal antiserum (Rb3443) directed against PSGL-1; these reagents have been previously described (28,40,41): both the polyclonal antisera and all monoclonal antibodies were raised against peptides representing the terminal 19 amino acids of mature PSGL-1, and with exception of PSL-275, all block the binding of P-selectin to PSGL-1. The binding epitope of PSL-275 is cleaved by mocarhagin (42), a protease which cleaves a ten amino acid fragment off the N-terminus of mature PSGL-1 which contains the P-selectin and L-selectin binding site of PSGL-1 (32,42). Phycoerythrin (PE)-conjugated goat anti-mouse IgG antibodies were obtained from Jackson ImmunoResearch Labs, Inc. Unconjugated anti-CD43 (leukosialin) MoAb L60 (which recognizes an O-linked sialic acid epitope (27)) and isotype control (IgG1) were from Becton-Dickinson. Hamster anti-rat L-selectin MoAb HRL-1 (a function blocking antibody) and hamster isotype control were from Pharmingen, and murine anti-human L-selectin MoAb LAM 1-3 and isotype control were from Coulter Corp.

Enzymes: O-sialoglycoprotease was obtained from Accurate Chemical. Bromelain, chymotrypsin and pronase were obtained from Sigma Chemical. Human neutrophil elastase was obtained from Athens Research. Porcine pancreas elastase was obtained from Elastin Products. Mocarhagin was obtained from Dr. Ray Camphausen (Genetics Institute). Recombinant phospholipase C (PI—PLC) was obtained from Oxford Glycosystems. V. Cholera neuraminidase, Newcastle neuraminidase, α-L-fucosidase, N-glycosidase F, Endoglycosidase H and recombinant Endoglycosidase F2 were obtained from Roche Molecular Biochemicals.

Immunofluorescence studies. Analysis of expression of PSGL-1 antigen and of PSGL-1 activity (binding of P-selectin-Ig) on KG1a and HL60 cell suspensions was performed by flow cytometry. KG1a or HL60 cell suspensions (1×10⁶ KG1a in 100 μl phosphate-buffered saline (PBS) containing 2% FBS on ice) were incubated with anti-PSGL-1 monoclonal antibodies (3 μg each) or with equivalent amounts of isotype control antibody in PBS/2% FBS for 30 minutes. After washing in PBS/2% FBS, the cells were treated with 5 μg PE-conjugated goat anti-mouse IgG secondary antibody, followed by additional washes in PBS/2% FBS. Flow cytometric analysis of PSGL-1 activity was performed by incubating cells with P-selectin-Ig or human IgG1 isotype control (each at 4 μg/10⁶ HL60 or KG1a cell) pre-complexed with protein A-FITC (Zymed) as described (41). For cell sorting, KG1a were stained with a cocktail of anti-PSGL-1 monoclonal antibodies or with P-selectin-Ig and populations were separated by fluorescence intensity into cells with highest and lowest PSGL-1 levels (top 5% and bottom 5%, respectively, of fluorescence intensity). Flow cytometry and cell sorting was performed on a FACStar^{PLUS} apparatus equipped with an argon laser tuned at 488 nm (Becton Dickinson).

Lymphocyte Adherence Assay. The Stamper-Woodruff lymphocyte adherence assay was performed on glutaraldehyde-fixed cyto-centrifuged (cytospin) preparations of cells as previously described (26), or on glutaraldehyde-fixed slides containing cell membrane protein preparations (see below). For all adherence assays, lymphocyte suspensions (10⁷/ml rat thoracic duct lymphocytes or human peripheral blood lymphocytes isolated from Ficoll density centrifugation of citrated blood) in RPMI 1640 medium were overlaid onto relevant glass slides containing cytospin preparations of KG1a or HL60 cells, containing membrane protein preparations, or containing glutaraldehyde-fixed 8-um-thick frozen sections of murine lymph node (lymph nodes consistently served as a positive control to assess the expected L-selectin-mediated lymphocyte binding to lymph node HEV). Slides were placed on a rotating platform for incubation under shear (80 rpm) at 4° C. for 30 minutes. Slides were then rinsed in PBS to remove nonadherent lymphocytes, fixed in 3% glutaraldehyde, and stained with methyl green-thionin. Slides were examined for lymphocyte adherence to cells or to spotted membrane protein material by light microscopy. In each Stamper-Woodruff assay, control assays were performed in parallel to verify L-selectin-specific lymphocyte adherence, these controls consisted of phorbol myristate acetate (PMA)-treatment of lymphocytes (which results in shedding of L-selectin), pre-incubation of lymphocytes with blocking anti-L-selectin moAb (either HRL-1 for rat or LAM1-3 for human lymphocytes) or with respective control isotype moAb, and assays performed in the presence of 10 mM EDTA, all as previously described (26).

For cytospin preparations, L-selectin ligand activity was measured by counting the number of lymphocytes adherent to a confluent area of cytocentrifuged cells, using an ocular grid under 100× magnification. For measurement of KG1a ligand activity following experimental interventions, untreated (control) cells and treated cells underwent adherence assays in parallel. Ligand activity of treated KG1a cells, expressed as the percent of lymphocyte binding compared to control cells, was assessed by counting respective adherent lymphocytes on several grid areas of KG1a cytospins, with minimum of three slides per experiment, in three separate experiments. For adherence assays to assess blocking activity of anti-PSGL-1 antibodies, KG1a cytospins were preincubated in RPMI 1640 containing either antibodies or isotype control antibody at concentrations as high as 50 μg/ml for 30 minutes at 4° C. Slides were then rinsed and cell suspensions containing the respective antibodies at concentrations equivalent to that used in the preincubation were overlaid onto the slides. For experiments using cytospin mixtures of HL60 and KG1a cells, immunohistochemistry for CD34 antigen was performed as per manufacturer's (Dako) recommendations following the adherence assay to identify KG1a cells (alkaline phosphatase, New Fuchsin chromagen; stained cells are pink).

For Stamper-Woodruff adherence assays on native or enzyme-treated preparations of cell membrane proteins, 1 μg of protein (prepared as described below) was spotted onto glass slides, allowed to dry at room temperature, then fixed in 3% glutaraldehyde in the usual manner (26). The adherence assays were performed on these slides exactly as described for cytospin preparations of cells, and the quantitation of lymphocyte binding to spotted material was performed by counting adherent lymphocytes on three fields utilizing an ocular grid under 100× magnification. For each enzyme treatment, controls consisted of buffer (alone) treatment, and lymphocytes adhering to buffer-treated and enzyme-treated preparations were quantified (minimum of three slides per experiment, three separate experiments). Ligand activity following enzyme treatments is expressed as percentage of lymphocyte adherence compared with that of buffer-treated membrane proteins.

Enzyme digestions. Cells in suspension (20×10⁶ cells/ml RPMI) were treated with proteases or with buffer alone (control) prior to cytocentrifugation. Protease concentrations for digestions were as follows: 100 U/ml chymotrypsin; 0.1% bromelain; 50 mg/ml human neutrophil elastase or porcine pancreas elastase; O-sialoglycoprotease at 0.24 mg/ml; mocarhagin at 10 μg/ml. All digestions were 30 minutes at 37° C. in RPMI 1640 medium, except for mocarhagin digestion for which cells were suspended in 0.15M NaCl, 2 mM CaCl₂, 1 mg/ml BSA, 0.01 M Tris, pH 7.4, for 20 minutes at 37° C. To assess the efficiency of O-sialoglycoprotease digestion and of mocarhagin digestion, cells were analyzed by flow cytometry for cleavage of an OSGP-sensitive epitope of CD34 (using QBEND10 MoAb, Coulter-Immunotech) and of the PSL-275 epitope of PSGL-1, respectively. Following digestions, cells were washed in RPMI 1640 medium then cytocentrifuged onto slides for adherence assays. For digestion with Newcastle virus neuraminidase, glutaraldehyde-fixed cytospins of KG1a cells were rinsed twice with enzyme buffer (50 mM NaAcetate, 154 mM NaCl, 9 mM CaCl₂₁ pH 5.5), and then incubated at 37° C. for one hour with 50 μl of buffer (control) or 10 mU/ml neuraminidase in buffer. Following the incubations, the slides were washed in RPMI 1640 with 1% FBS and the lymphocyte adherence assay was performed on the cytospins.

For α-L-fucosidase, N-glycosidase F, Endoglycosidase F2 or Endoglycosidase H digestions, KG1a membrane preparations (as described below) were denatured in 1 % SDS and boiled five minutes. Denatured membrane preparations were then diluted in buffers (as noted in parentheses) specific for each glycosidase and incubated for 24 hours at 37° C. in either 0.8 U/ml α-L-fucosidase (0.1 M NaAcetate, pH 5.5), 8 U/ml N-glycosidase F (50 mM NaPhosphate, 12.5 mM EDTA, 1% 2-mercaptoethanol, pH 7.5), 4 mU/ml Endoglycosidase F2 (0.5M NaAcetate, 1% NP-40, pH 5.5) or 50 mU/ml Endoglycosidase H (0.1M NaAcetate, 1% 2-mercaptoethanol, 0.5 mM PMSF, pH 5.5). Confirmation of enzyme activity was made by analysis of shifts in SDS-PAGE protein profiles (Coomassie staining) of enzyme-treated versus buffer-treated preparations. Following buffer treatments or enzyme digestions, all suspensions were normalized to a protein concentration of 0.2 mg/ml, and 5 μl (1 μg total protein) of each suspension was then spotted onto slides for adherence assays as described above.

Analysis of Ligand Re-expression Following Neuraminidase or Bromelain Treatment of KG1a Cells. KG1a (20×10⁶ cells/ml) were treated with 0.1 U/ml neuraminidase (V. Cholerae Neuraminidase, 1 U/mi stock, Boehringer Mannheim, Indianapolis, Ind.) in RPMI 1640 without bicarbonate (Gibco/BRL; pH 7.4) for one hour at 37° C. For controls, an equivalent volume of enzyme buffer (one-tenth volume of 50 mmol/L sodium acetate, 154 mmol/L NaCI, 9 mmol/L CaCl₂, pH 5.5) was added to KG1a in RPMI 1640 without bicarbonate (pH 7.4) under identical conditions (final pH of solution was 6.8). Bromelain digestion was performed as described above. Following neuraminidase or bromelain digestion, cells were washed three times in RPMI 1640 without bicarbonate and an aliquot, designated as t=0, was tested in the adherence assay to verify complete loss of ligand activity. The remaining cells were cultured in RPMI 1640 with 10% FBS and an aliquot of these cells removed at serial time points over 24 hours for analysis of the reappearance of ligand activity in the adherence assay.

Effects of Metabolic Inhibition of N-linked Glycosylation or Protein Synthesis. Tunicamycin (Sigma Chemical Co., St. Louis, Mo.; prepared as 5 mg/ml stock in DMSO) was added to cell cultures (15 μg/ml final concentration, 10⁷ cells/ml in RPMI 1640 with 10% FBS) following either V. Cholera neuraminidase treatment (0.1 U/ml) or respective enzyme buffer treatment of the KG1a cells for one hour as described above. As control for the effects of DMSO, parallel cultures were established in the presence of 0.3% DMSO. For metabolic studies utilizing either swainsonine (stock 0.5 mg/mi in PBS) or deoxymannojirimycin (stock 10 mg/ml in PBS), cells were treated with neuraminidase and then washed and suspended in RPMI 1640/10% FBS containing either 0.4 mg/ml deoxymannojirimycin or 40 μg/ml swainsonine. All cells were incubated in the presence of metabolic inhibitors of glycosylation for 24 hours, and cells were washed then cytocentrifuged onto glass slides for analysis of L-selectin-dependent lymphocyte adherence.

For studies on the effect of protein synthesis inhibition, cycloheximide (Sigma) (endotoxin-cleared (27)) was added to cultures of neuraminidase-digested, bromelain-digested or buffer-treated KG1a cells at 1.25 μg/ml final concentration, 10⁷ cells/ml in RPMI 1640 with 10% FBS. In each case, cells were cultured for 20 hours and the expression of L-selectin ligand activity was measured by adherence assay at the end of the culture period. For all studies utilizing metabolic inhibitors, parallel cultures were established in the presence of ³⁵S-methionine/cysteine and protein synthesis was measured by scintillation counting of trichloroacetic acid (TCA)-precipitable protein as previously described (27).

Salt and pH Treatments of KG1a Cells. KG1a cells (50×10⁶ cells) were incubated for five minutes at 37° C. with vigorous agitation in 2 ml of high salt solution (1M NaCl) under a variety of pH conditions: 0.1M Tris pH 9; 0.1M NaPhosphate pH 7; 0.1M NaAcetate pH 5; and 0.1M acetic acid, pH 3. Following the buffer treatments, cells were centrifuged and supernatants collected. Cells were then suspended in RPMI1640 medium, washed two times and the cells and supernatants were assessed for ligand activity. Supernatants were spotted onto glass slides, air-dried, and glutaraldehyde treated in the usual fashion for Stamper-Woodruff assays. L-selectin ligand activity of the cells and supernatants was compared to cells similarly treated with agitation in RPMI1640 medium alone.

Isolation of KG1a Membranes and Organic Solvent Extraction of Membranes. KG1a membranes were isolated by nitrogen cavitation followed by differential centrifugation of cellular contents to isolate membrane fractions (43). Approximately 4×10⁹ cells were washed twice in PI buffer (150 mM NaCl, 50 mM Tris-HCl pH 7.4, 1 mM EDTA, 0.02% Na Azide, containing protease inhibitors (20 μg/ml PMSF, 0.5 μg/ml Aprotinin, 0.5 μg/ml Pepstatin A, 0.5 μg/ml Leupeptin, 20 μg/ml Trypsin inhibitor)) then resuspended at 4×10⁸ cells/ml. The cell suspension was subjected to nitrogen cavitation (400 psi), and ruptured cells were then centrifuged 15 minutes at 3,600× g to pellet nuclear and mitochondrial debris. The supernatant was saved, and the pellet was washed with PI buffer and centrifuged as above. The two supernatants were then pooled and centrifuged at 22,000× g to pellet membrane material that was then suspended in 1 ml PI buffer.

For lipid extractions, membrane preparations (0.2 mg/ml protein) were mixed (1:5) with organic solvents: water-saturated butanol or chloroform: methanol (2:1) for three minutes. Appropriate solvent: PBS mixtures (1:1) were then added at 10% of the above final volume and phases were separated by centrifugation. Aqueous and organic phases were then evaporated, and resulting material was resuspended in original volume of PBS and spotted on glass slides for analysis in the Stamper-Woodruff assay.

Results

PSGL-1 is Not the Principal L-Selectin Ligand on KG1a Cells

Utilizing the Stamper-Woodruff assay, in every case where observed, lymphocyte adherence to cytospin preparations of cells or to membrane material spotted on glass slides was absolutely L-selectin specific, as verified by complete elimination of lymphocyte adherence following PMA-treatment of lymphocytes, preincubation of lymphocytes with anti-L-selectin monoclonal antibody and by performing the assay in the presence of EDTA. Incubation of KG1a cytospin monolayers with anti-PSGL-1 monoclonal antibodies singly and in combination, including PL-1, 4F9, 4D8 and 2G3 (each of which are functional blocking moAbs and attach to the PSGL-1 N-terminal binding determinants for both L- and P-selectin), did not inhibit lymphocyte attachment in the adherence assay (Table 1). These incubations were performed both before and after glutaraldehyde fixation of KG1a onto glass slides, with antibody concentrations as high as 100 μg/ml, both singly and in combination. Moreover, incubation of KG1a with undiluted anti-PSGL-1 rabbit antisera Rb3443 did not interfere with L-selectin ligand activity in the adherence assay. In each case where antibodies were utilized, KG1a cells were stained with the respective antibody and isotype (or preimmune sera) control and analyzed by flow cytometry to confirm attachment to PSGL-1. HCELL activity was also unaffected by preincubation of KG1a with P-selectin-Ig chimera (Table 1).

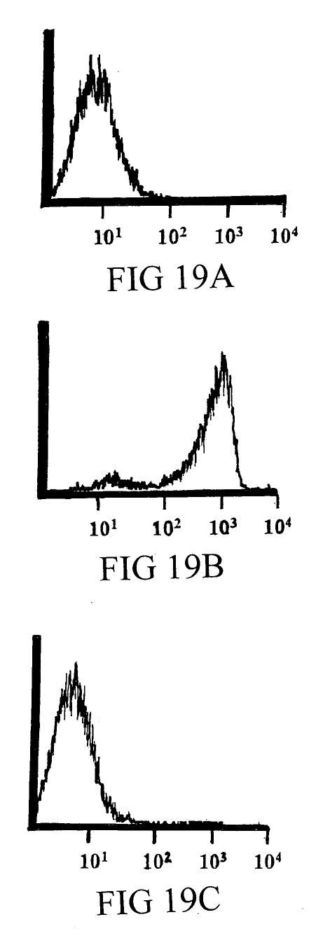

Mocarhagin digestion abrogates both P-selectin and L-selectin binding activity of PSGL-1 on neutrophils and HL60 cells (32). KG1a cells were incubated with this protease and then analyzed by flow cytometry for P-selectin binding by indirect staining utilizing P-selectin-Ig chimera and FITC-conjugated protein A. As shown in FIG. 19, mocarhagin digestion completely eliminated P-selectin binding to PSGL-1. Staining of KG1a cells with N-terminal-specific moAb PSL-275 was similarly eliminated following mocarhagin treatment, confirming the high efficiency of digestion of the N-terminal region of PSGL-1 by this protease. However, following incubation with either mocarhagin or enzyme buffer alone (control), L-selectin-specific lymphocyte adherence to KG1a was unaffected in the Stamper-Woodruff assay (Table 2). OSGP digestion of KG1a also eliminated P-selectin-Ig binding to KG1a cells, without affecting HCELL activity (Table 2).

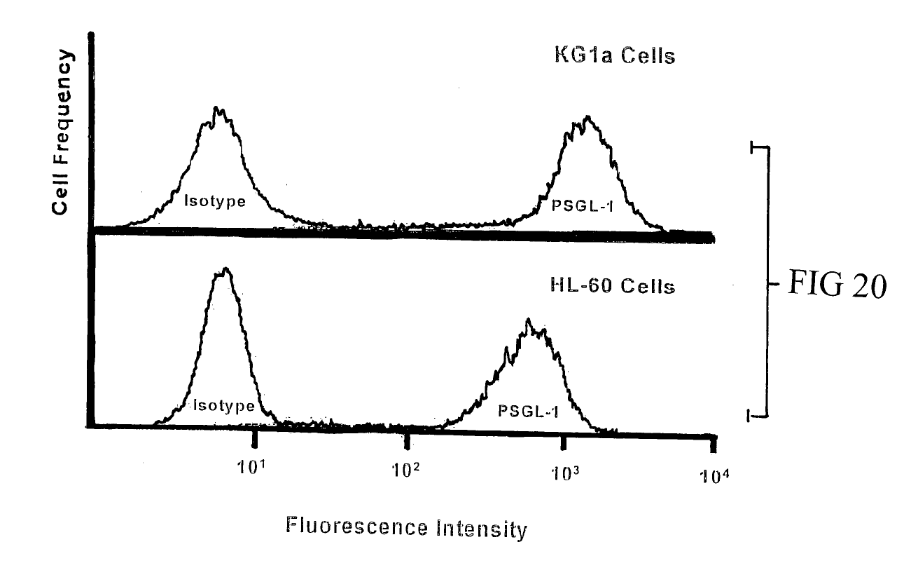

The hematopoietic cell line HL60 has been utilized extensively for studies of PSGL-1 structure and function (28,29,34,37,40,44). Indeed, PSGL-1, as expressed specifically by HL60, has been reported to be an L-selectin ligand (30,31). As measured by binding of P-selectin-Ig chimera, KG1a express functional levels of PSGL-1 comparable to HL60 cells (FIG. 20A). However, in Stamper-Woodruff assays of cytospin mixtures of KG1a and HL60 cells, lymphocytes bind only to KG1a cells (pink staining cells, FIG. 20B). The selectivity and specificity of binding to KG1a in mixing studies indicate that the absence of lymphocyte adherence to HL60 cells under Stamper-Woodruff shear assay conditions is not due to an indirect biological effect of HL60 on lymphocytes rendering them incapable of attaching to L-selectin ligands (such as activation-induced shedding of L-selection (45,46)), as any such effect would concomitantly prevent binding to KG1a in these cytospin mixtures.

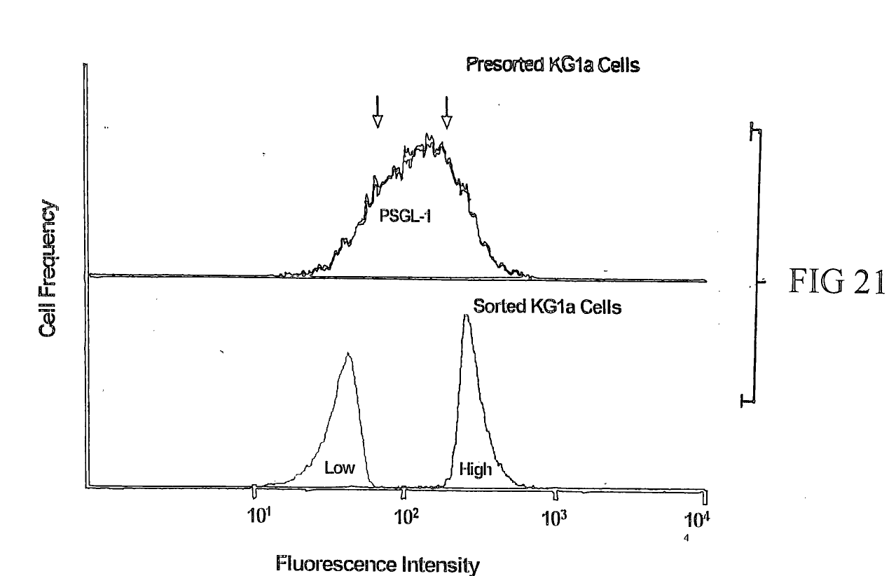

To further examine the role of PSGL-1 in the L-selectin ligand activity of KG1a cells, KG1a cells were separated into subpopulations with high- and low-level expression of PSGL-1 by fluorescence-activated cell sorting utilizing a cocktail of anti-PSGL-1 antibodies. Cytospin preparations of the separated populations were made and cells were then analyzed for L-selectin ligand activity in the Stamper-Woodruff assay. The adherence of lymphocytes was identical in the two populations (data expressed as mean (SEM) percent of binding of presorted KG1a control: “low” PSGL-1 was 100.6 (9.5) and “high” PSGL-1 was 98.6 (11.1)), despite high efficiency separation of PSGL-1 expressing cells as verified by repeat staining using anti-PSGL-1 antisera (FIG. 21).

HCELL is an Integral Membrane Protein.

As shown in Table 2, treatment of KG1a cells with proteases such as bromelain, chymotrypsin, and pronase completely abrogates L-selectin ligand activity, while digestion with pancreas and neutrophil elastase partially affects activity and digestion with mocarhagin and O-sialoglycoprotease has no effect on ligand activity. Ligand activity is also resistant to digestion with PI—PLC (Table 2). Cycloheximide treatment of cells for as long as 20 hours following protease (bromelain) digestion completely prevents ligand re-expression without significantly affecting cell numbers or viability (trypan blue exclusion >95%) (Table 3). Taken together, the results of these experiments indicate that the KG1a ligand is a non-mucin protein whose expression requires de novo protein synthesis rather than translocation of preformed, stored molecules from intracellular compartments to the membrane.

To determine whether the ligand is an integral membrane structure, KG1a cells were treated with high salt conditions over a range of pH levels, and supernatant (elution) and residual membrane fractions were then tested in the Stamper-Woodruff assay. L-selectin-specific adherence of lymphocytes was not observed on slides containing supernatant fractions, but was found on the cell membranes following all treatments, even on membrane ghosts resulting from lysis of the cells in low ionic conditions. In order to examine whether membrane lipid structures contained any ligand activity, organic solvent extractions of cells were performed, and respective organic and aqueous phases were then tested in the Stamper-Woodruff assay. In each case, ligand activity partitioned in the aqueous phase, and there was no activity within the lipid (solvent) compartment.

HCELL L-Selectin Binding Determinant(s) are Sialofucosylglycoconjugates Presented on Complex N-Linked Glycans.

Early studies showed that treatment of KG1a cells with V. Cholera neuraminidase completely abrogated HCELL binding activity (24). This enzyme has broad specificity for cleavage of terminal sialic acids in either α2-3, α2-6 or α2-8 linkage to relevant sugar residues in oligosaccharides. To further characterize the sialic acid linkage, the Newcastle disease virus neuraminidase that cleaves terminal sialic acids in α2-3 and α2-8 linkages, but not in α2-6 linkage (47,48) was utilized. As with results of V. Cholera neuraminidase digestions, treatment of KG1a cells with Newcastle virus neuraminidase prior to the Stamper-Woodruff assay abrogated HCELL binding activity (mean binding of 9.8% (SEM of 2.2%) compared with respective buffer treatment alone, p<0.001 by paired t test). These data confirm that sialylation is a critical functional modification of HCELL and, more specifically, indicate that L-selectin binding determinant(s) on HCELL consist of oligosaccharides principally bearing α2-3- and/or α2-8-linked sialic acids. To examine whether fucosylation is required for ligand activity, denatured KG1a membrane preparations were digested with α-L-fucosidase, which cleaves terminal fucose residues in α1,2-, α1,3- and α1,4-linkages. Treatment with this enzyme markedly diminished HCELL binding activity (mean binding of 11.5% (SEM of 2.9%) compared with respective buffer treatment alone, p<0.001 by paired t test). Collectively, the results of sialidase and fucosidase digestions indicate that the relevant binding determinant of HCELL is a sialylated, fucosylated glycoconjugate.

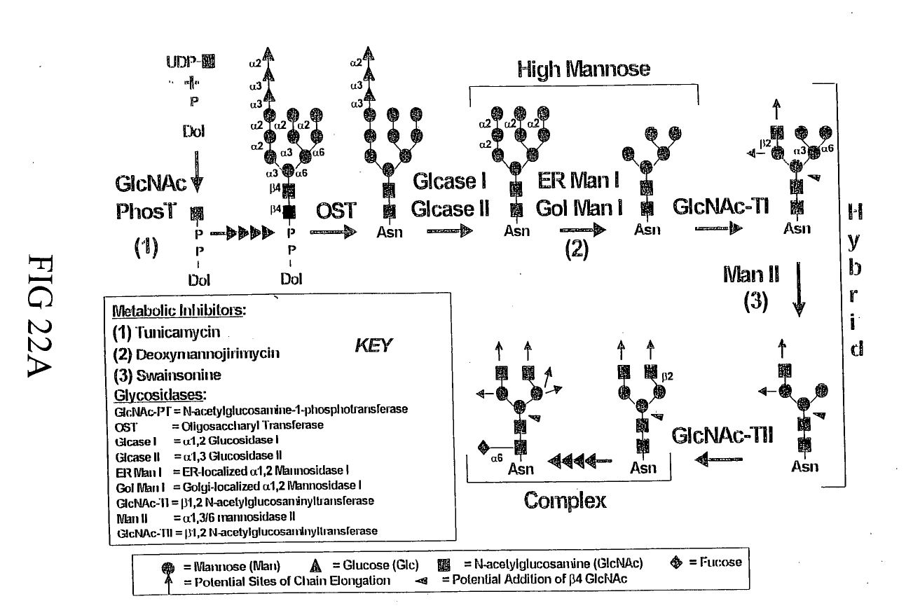

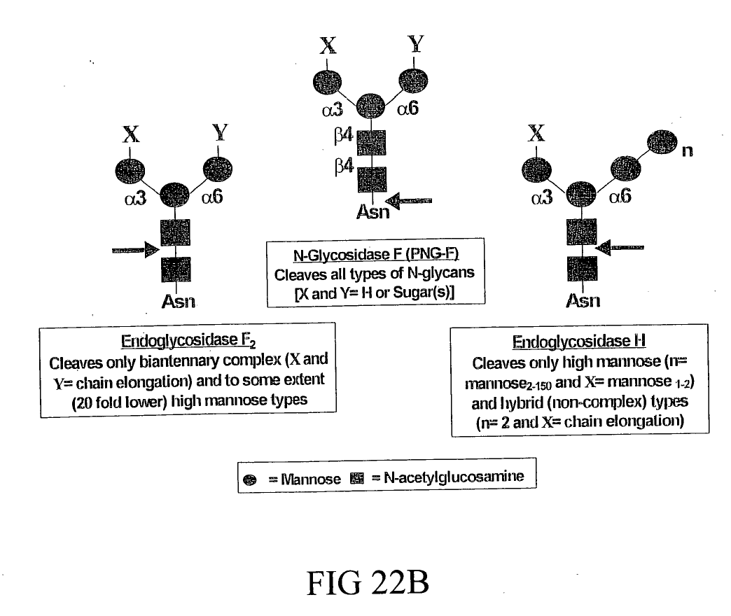

Following neuraminidase treatment of KG1a cells, HCELL binding activity reappears within twenty hours of culture (27). As noted above, inhibition of protein synthesis over a twenty-hour period following either neuraminidase or protease treatment prevents ligand re-expression (Table 3). Collectively, these data indicate that ligand re-expression following such treatments results from de novo glycoprotein synthesis and processing. Thus, incubation of the cells with metabolic inhibitors of glycosylation during the period of ligand re-expression would result in replacement of original membrane ligand glycoprotein(s) with ligand molecules affected by the relevant metabolic treatments. Accordingly, to investigate the effect of modifications in N-linked oligosaccharide processing on the activity of HCELL, KG1a cells were treated with V. Cholera neuraminidase, and these treated cells were cultured for twenty-hours in the presence of glycosylation inhibitors, tunicamycin, swainsonine and deoxymannojirimycin (see FIG. 22A for schema depicting the effects of these inhibitors on the different stages of carbohydrate processing). Before culturing the cells, the cleavage of sensitive sialic acid epitopes was confirmed in every case by testing for loss of binding activity in the Stamper-Woodruff shear assay and by measuring the expression of the sialic acid-dependent L60 epitope of CD43 by flow cytometry (27). For each inhibitor, effects on glycosylation of membrane proteins were verified by analysis of protein band shifts by SDS-PAGE of membrane lysates, however, treatment of cells with the glycosylation inhibitors did not affect cell viability (trypan blue exclusion >98% for each inhibitor).

As shown in Table 4, treatment with glycosylation inhibitors without prior neuraminidase digestion did not affect L-selectin ligand activity of KG1a cells, suggesting that the membrane turnover of fully processed L-selectin ligand is relatively slow and there is no direct toxic effect of the inhibitors on the activity of the L-selectin ligand already expressed on the membrane. However, following neuraminidase digestion, tunicamycin treatment completely prevented ligand re-expression and both swainsonine and deoxymannojirimycin markedly diminished expression of ligand activity. Return of the L60 epitope of CD43 was not inhibited by treatments with either tunicamycin, swainsonine or deoxymannojirimycin, indicating that these agents did not affect sialylation directly and, in particular, did not affect synthesis of O-linked sialylated structures. Moreover, similar to findings in previous studies (27), these metabolic agents had no effect on de novo protein synthesis as TCA-precipitable ³⁵S-methionine/cysteine radiolabeled protein counts were similar to that of cells not treated with the inhibitors.

To further analyze the structural biology of the critical glycosylations conferring ligand activity, Stamper-Woodruff assays were performed on N-glycosidase and buffer (control) treated KG1a membrane preparations. Interestingly, HCELL binding activity was maintained following SDS solubilization and 2-mercaptoethanol treatment of membrane proteins, indicating that the protein scaffold can be denatured significantly without disturbing ligand activity. FIG. 22B displays the relevant specificity for each endo-N-glycosidase. As shown in Table 5, PNG-F digestion completely abrogates HCELL activity, whereas Endo F2 digestion moderately affects activity and Endo H digestion has no effect.

Discussion

Previous studies from the laboratory have identified an L-selectin ligand naturally expressed on the human hematopoietic progenitor cell line KG1a and on normal human CD34+ hematopoietic progenitor cells. This ligand, designated HCELL, is distinguished from its endothelial counterparts by: (1) Displaying sulfation-independent binding activity; (2) Resistance to inactivation by OSGP digestion; and (3) Absence of the MECA79 antigen (26,27). The present study was undertaken to characterize the structural biology of the binding determinant directing the adherence of L-selectin to this novel ligand. The data presented here indicate that HCELL is a membrane-bound glycoprotein which is biochemically distinct from PSGL-1 and is unique among naturally-expressed L-selectin ligands in that the L-selectin binding determinant(s) are presented on N-linked complex glycans.

As a family of molecules, the selectins possess a unifying characteristic of adhering to ligands containing a common carbohydrate motif consisting of a sialylated, fucosylated epitope, the prototype of which is sialylated Lewis X (15). Whereas L-selectin and E-selectin appear to have multiple ligands (49), P-selectin recognizes a single ligand, PSGL-1, which is expressed on hematopoietic progenitor cells and on mature myeloid and lymphoid cells (34-36). PSGL-1 is a heterodimer consisting of two identical disulfide-linked subunits of 120,000 m.w., and is unique among all the selectin ligands in that it functions as a ligand for all three selectins (50). The binding determinant for P-selectin is localized to the amino terminal portion of the molecule, in a site which is sensitive to digestion by OSGP (51) and by mocarhagin (42), and P-selectin binding to PSGL-1 is dependent on sulfation of tyrosines within this region (52). The E-selectin binding determinant of PSGL-1 is physically remote from the N-terminus and is not sensitive to OSGP-digestion nor dependent on N-terminal sulfation (50,53), whereas the L-selectin binding determinant overlaps that of P-selectin. It is localized to the N-terminal portion of the molecule, it is sulfation-dependent, and OSGP and mocarhagin digestion each render PSGL-1 incapable of binding L-selectin (31-33). For binding of L-selectin to PSGL-1 and to its ligands expressed on endothelial cells, sulfation is also a critical modification, but this sulfation is required on the N-terminal tyrosines in PSGL-1 whereas it is on carbohydrates for endothelial ligands (18, 54-56).

Although data from previous studies suggest distinction of HCELL from PSGL-1 based on HCELL's sulfation-independent, OSGP-resistant L-selectin binding activity, KG1a cells express abundant levels of PSGL-1 and the previous studies did not directly address the relationship of HCELL to PSGL-1. Indeed, there is precedent for structural heterogeneity in PSGL-1 conferring selective selectin binding. E-selectin binding to PSGL-1 is known to occur on a subset of specifically decorated PSGL-1 containing the CLA epitope, a post-translational carbohydrate modification recognized by the monoclonal antibody HECA-452 (57). HCELL activity, as identified in the shear-based Stamper-Woodruff assay, was attributable to PSGL-1. The data reported here clearly demonstrate that the L-selectin binding activity of HCELL is independent of PSGL-1: (1) Mocarhagin and OSGP digestion were each completely effective in cleaving the L-selectin/P-selectin binding domain of the PSGL-1 molecule (as evidenced by complete abrogation of P-selectin-Ig binding), but had no effect on HCELL activity; (2) Experiments utilizing mixtures of KG1a cells and HL60 cells (which express PSGL-1 in levels equivalent to KG1a) reveal that only KG1a cells support L-selectin-dependent lymphocyte attachment in the Stamper-Woodruff assay; (3) Antibody blocking studies utilizing both monoclonal and polyclonal reagents directed against UP-selectin binding epitopes of PSGL-1 did not affect L-selectin-mediated lymphocyte binding of KG1a; and (4) No difference in L-selectin-mediated lymphocyte adherence was observed in KG1a cells sorted for high and low levels of PSGL-1 expression. Differences in lymphocyte binding would have been observed in the sorted populations if PSGL-1 were the molecule conferring HCELL binding activity, as the conditions of the Stamper-Woodruff assay are optimized to measure subtle differences in ligand activity. It is unlikely that a particular epitope of PSGL-1 was selected in sorting studies, because a cocktail of monoclonals and a polyclonal antiserum were each used for these experiments, and the efficiency of sorting was consistently verified by utilizing the monoclonal cocktail (for sorting based on the polyclonal antiserum) or the polyclonal antiserum (for sorting based on the monoclonals). These sorting data, in combination with the mixing studies and enzymatic digestions, present strong evidence that PSGL-1 as expressed on either KG1a or HL60 cells does not contribute to the L-selectin ligand activity observed in the experiments utilizing the conventional Stamper-Woodruff shear-based L-selectin binding assay.

Though apparently in contrast to results of previous studies (30-33), the finding that HCELL binding activity is not attributable to PSGL-1 does not negate a role for PSGL-1 as an L-selectin ligand. Rather, the present data suggest that the biophysical requirements for PSGL-1 -specific L-selectin binding on KG1a cells and HL60 cells are not met under the operative conditions of the Stamper-Woodruff assay. The appropriate engagement of L-selectin to its ligands is critically dependent on biophysical shear forces in order to both promote and maintain L-selectin/L-selectin ligand adhesive interactions (58). Many studies directed at identifying L-selectin ligands have utilized static assay conditions, and have relied heavily on molecular probes such as L-selectin-Ig chimeras, presented in non-physiologic conditions such as in high concentrations in solution or as displayed on artificial substrates. Compared to these approaches, the Stamper-Woodruff assay offers an important advantage in that it measures functional adhesive interactions under shear conditions of native L-selectin, as expressed naturally on the membrane of a physiologic cell type (the lymphocyte), with its relevant ligand(s) expressed in their native state in natural lipid bilayers of cell membranes.

A variety of selectin ligands have been described, including membrane glycoproteins and glycolipids, secreted glycoproteins and even extracellular matrix elements (15,49). The data presented here show that HCELL is a membrane glycoprotein. Taken together, the results of neuraminidase and fucosidase treatments, of protease digestions and of metabolic inhibition of both protein and glycoconjugate synthesis indicate that the relevant HCELL carbohydrate binding determinants are sialofucosylglycoconjugates expressed on a protein core. The partitioning of the activity in the aqueous phase of organic solvent extractions argues strongly against a significant component of glycolipid to HCELL activity. Moreover, this glycoprotein appears to be an integral membrane structure, as it is resistant to PI-PLC digestion and ligand activity is intact and resistant to elution following treatment of cells with high salt buffers over a wide pH range.

All previously described naturally expressed endothelial L-selectin ligands are sialomucins, and the requisite sialylated L-selectin binding determinant(s) are expressed on O-linked (i.e., glycan attached to serine or threonine residues on the protein core) structures that are sensitive to OSGP digestion (59,60). In addition, PSGL-1 is a sialomucin, and the carbohydrate determinant(s) mediating L- and P-selectin binding are expressed on O-linked structures that are also sensitive to OSGP digestion (31-33,51). The finding that HCELL binding activity is resistant to OSGP digestion (26) suggests that HCELL may not be a sialomucin, but does not provide direct information on the structure of the relevant carbohydrate-protein linkage. The results presented here provide two independent lines of evidence, obtained from separate but corroborating experimental approaches (N-glycosidase digestions and treatment of cells with metabolic inhibitors of glycosylation), which show that critical L-selectin binding determinants on HCELL are presented on N-linked (i.e., attached to asparagine residues on the protein core) glycoconjugates. The diminished re-expression of ligand activity observed here in the presence of glycosylation inhibitors (following desialylation) was not due to a generalized inhibition of sialylation since the L60 epitope of CD43 was reexpressed completely in the presence of these agents. Moreover, it was not due to a generalized inhibition of protein synthesis of the cells, as ³⁵S-methionine/cysteine incorporation into nascent protein (assessed by measurement of TCA-precipitable radioactive protein) was not significantly changed in the presence of any of the N-linked glycosylation inhibitors.

The relevant N-linked glycan modification of HCELL which contains the L-selectin binding determinant(s) is, at minimum, a biantennary complex (i.e., modified at both Man-1,6 and Man-1,3 arms) structure (see FIG. 4A). N-glycanase hydrolyzes all types of N-glycan structures in mammalian glycoproteins (61), whereas the substrate specificity of Endo H is high mannose and hybrid structures (61,62) and Endo F2 hydrolyzes only complex biantennary and high mannose structures (62,63) (see FIG. 4B). The observation that Endo F2 and N-glycanase digestions significantly affect HCELL binding activity while Endo H digestion has no effect on HCELL activity excludes unmodified high mannose and hybrid structures from consideration and, in particular, indicates that biantennary complex and multiantennary complex structures present the relevant L-selectin binding. determinant(s). Moreover, the finding that metabolic treatments with swainsonine and deoxymannojirimycin each inhibit recovery of HCELL binding activity following neuraminidase treatment of the cells is also consistent with a minimum requirement for a biantennary complex structure (FIG. 4A). Specifically, the inhibition by swainsonine suggests that modification of the Man-1,6 arm (in addition to the Man-1,3 arm) is crucial for full expression of ligand activity (see FIG. 22A). Furthermore, these data underscore the finding that HCELL is a glycoprotein, as these enzymes and metabolic agents have no effect on glycolipid structures. Of note, HCELL activity withstands protein denaturation in 1% SDS and 1 % 2-mercaptoethanol (as used in the glycosidase buffers), suggesting that the core protein functions predominantly as a scaffold for the relevant ligand-specific carbohydrate modifications and that these N-linked carbohydrates direct the conformational/structural specificity for binding activity.

The results of this study offer important new insights into the complexity and breadth of L-selectin ligands. The data presented here provide first evidence of a native L-selectin ligand with N-glycan-dependent binding activity. This L-selectin ligand is expressed among hematopoietic progenitor cells in the bone marrow (2), raising further questions on the function of selectins and their ligands in hematopoiesis. The ordered expression of L-selectin and of HCELL and other L-selectin ligands (e.g., PSGL-1) among hematopoietic progenitors within discrete marrow microenvironmental “niches” will likely influence the differentiation and proliferation of hematopoietic cells (5), and, notably, recent data show that binding of P-selectin to PSGL-1 expressed on CD34+ human hematopoietic progenitor cells suppresses hematopoiesis (6). Consistent with a role for PSGL-1 as an inhibitor of hematopoiesis, increased myelopoiesis is observed both in fucosyltransferase VII-deficient mice (64) and in E- and P-selectin double-deficient mice (65). Though it appears that L-selectin knockout mice have no deficits in hematopoiesis (66), no systematic study of the clonogenic activity of L-selectin knockout progenitors or of the histology of the bone marrow has been performed in these animals.

Studies in vitro indicate that L-selectin expression is associated with higher clonogenic activity of human hematopoietic progenitors (7,12,13). Moreover, clinical studies indicate that L-selectin expression is associated with more rapid neutrophil and platelet engraftment following hematopoietic stem cell transplantation (8,9), but it is currently unclear whether this effect is due to improved homing of progenitors into the bone marrow and/or due to L-selectin-mediated effects on proliferation and/or differentiation of progenitors within the marrow. Because of their enhanced capacity to confer shear-resistant adherence, the expression of L-selectin ligands among relevant cells within the bone marrow may impact significantly on the initial seeding and maintenance of primitive progenitors within the cytoarchitecture of the hematopoietic microenvironment following stem cell transplantation. Consistent with this, recent studies show that the rolling efficiency of normal human bone marrow and fetal liver hematopoietic cells on immobilized L-selectin in flow chamber studies is greatest in the more immature subpopulation of cells (38). These studies also show that anti-PSGL-1 mAb PL1 has no significant effect on hematopoietic progenitor cell rolling on L-selectin over a wide range of shear forces (38), pointing to a role for non-PSGL-1 L-selectin ligands (such as HCELL) in mediating the binding of primitive hematopoietic cells to L-selectin.

More specifically, FIG. 19 shows a flow cytometric analysis of P-selectin-Ig binding to GL-1 on KG1a cells following mocarhagin digestion. The staining pattern of isotype control (human IgG1) antibody followed by protein A-FITC is depicted in FIG. 19A. FIG. 19B depicts the buffer-treated cells stained with P-selectin-Ig chimera followed by protein A-FITC. FIG. 19C shows the Mocarhagin-treated cells stained with P-selectin-Ig followed by protein A-FITC. Note the shift in fluorescence intensity of P-selectin-Ig binding to PSGL-1 following mocarhagin digestion, compared with buffer-treated cells.

FIG. 20 shows the flow cytometric analysis of PSGL-1 levels on KG1a cells and HL60 cells. Data shown are results obtained using P-selectin-Ig to stain cells, and therefore are reflective of PSGL-1 functional levels. Note that PSGL-1 expression is characteristic of both HL60 and KG1a cells. FIG. 20 shows that the L-selectin-mediated adherence of lymphocytes occurs on KG1a cells but not HL60 cells. This is representative of the result of lymphocyte adherence assay performed on cyto-spin mixture of KG1a cells and HL60 cells. Immunohistochemical staining for CD34 antigen (pink stain) identifies KG1a cells. As demonstrated by arrows, note that lymphocytes (solid blue dots) adhere only to pink (KG1a) cells (background stain is methyl green-thionin, 250× magnification).

FIG. 21 shows L-selectin-mediated lymphocyte adherence to PSGL-1-deficient KG1a cells. KG1a cells were sorted by FACS into populations (separated at arrows, upper panel) representing high level PSGL-1 expression (“high”) and low level PSGL-1 expression (“low”), as shown in lower panel. Adherence assays were performed on cells from both populations. L-selectin ligand activity was equivalently high in both cell populations (see text for details).

FIG. 22 shows a scheme of N-Glycan biosynthesis including assembly, processing and the synthesis of high mannose, hybrid and complex-type N-glycans and the sites of inhibition of N-glycan synthesis and processing by Tunicamycin (1), Deoxymannojirimycin (2) and Swainsonine (3). FIG. 22 shows the N-Glycan branch specificity of Endo-N-glycosidases. The specificities of N-glycan cleavage for N-glycosidase F (N-glycanase), Endoglycosidase F2 and Endoglycosidase H are as shown by arrows.

### Example 10

Selectin-mediated interactions are critical not only for the rapid and efficient recruitment of leukocytes at a site of injury (1), but for steady state, tissue-specific homing as illustrated in: (1) immunosurveillance of human skin-homing T cells (2, 3) and (2) human hematopoietic progenitor cells entry into the bone marrow (4-6). These two paradigms represent physiologically relevant processes that, at least in part, are dependent on the cell adhesive interactions between E-selectin and its leukocyte E-selectin ligands. There is convincing evidence suggesting that endothelial E-selectin as constitutively expressed on post-capillary venules in skin and bone marrow microvasculature is a key regulator and stimulus for leukocyte trafficking (7,8). The cell specific expression of leukocyte E-selectin ligands, is, therefore, important to understanding tissue tropisms, but the understanding of the identity and structure of these molecules in humans is incomplete.

One of the few leukocyte E-selectin ligands characterized to date is the 220 kDa homodimer, P-selectin glycoprotein-1 (PSGL-1). Its structure as a highly O-sialyated homodimeric molecule with sulfated tyrosines at the N-terminus also imparts its function as an L- and P-selectin ligand (9,10). When decorated with the appropriate silylated, fucosylated complex carbohydrates (i.e. sialyl Lewis X-like structures) that are distinctively detected by the rat monoclonal antibody, HECA-452, its expression on subsets of memory T-cells confers the ability of these cells to enter cutaneous vasculature through interactions with E-selectin (11-14), thereby bestowing its designation as cutaneous lymphocyte antigen (CLA). PSGL-1 lacking HECA-452 epitope(s) is widely expressed on other non-skin homing lymphocytes and myeloid cells (11, 15); expression of CLA is conferred by the appropriate posttranslational modifications necessary for generating the carbohydrate binding determinants requisite for E-selectin binding.

Since PSGL-1/CLA expression, as detected by HECA-452, is unique among skin-homing T-cells and CLA expression is functionally-correlated with E-selectin ligand activity required for lymphocyte trafficking to cutaneous vasculature, it was hypothesized that CLA expression on hematopoietic progenitor cells (HPC) would also confer the capacity of these cells to interact with E-selectin and traffic to the bone marrow (and/or skin). In this study, the expression of CLA or HECA-452-reactive membrane proteins on human leukemia cell lines and on circulating blasts from patients with acute leukemias was investigated. To this end, the identification of (1) several non-PSGL-1 molecules expressed on HPCs that possess E-selectin ligand activity and more specifically, (2) that a specialized HECA-452-reactive species of CD44 expressed on the human hematopoietic cell line, KG1a, and on de novo acute leukemias functions as an E-selectin ligand is set forth.

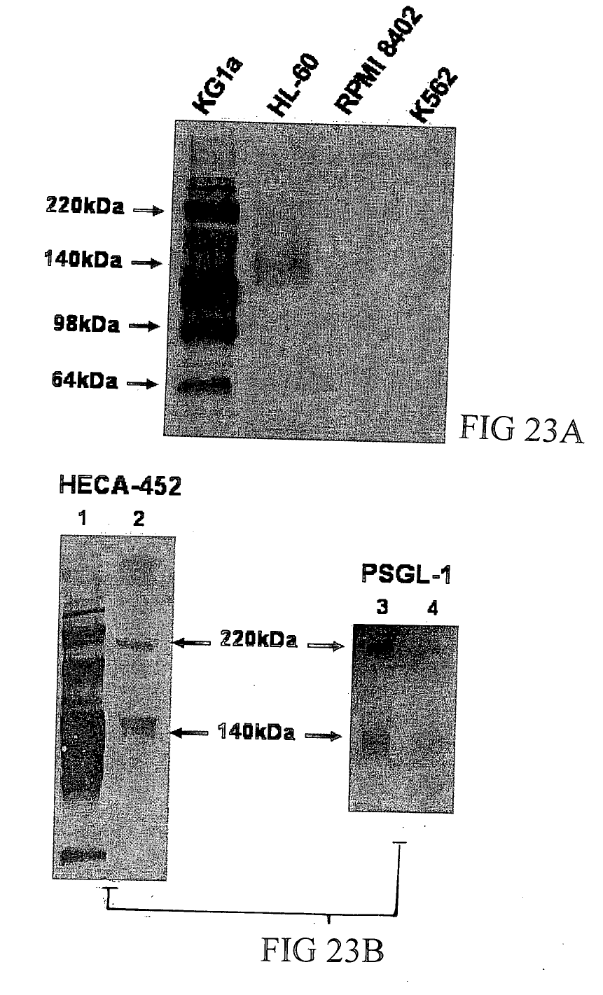

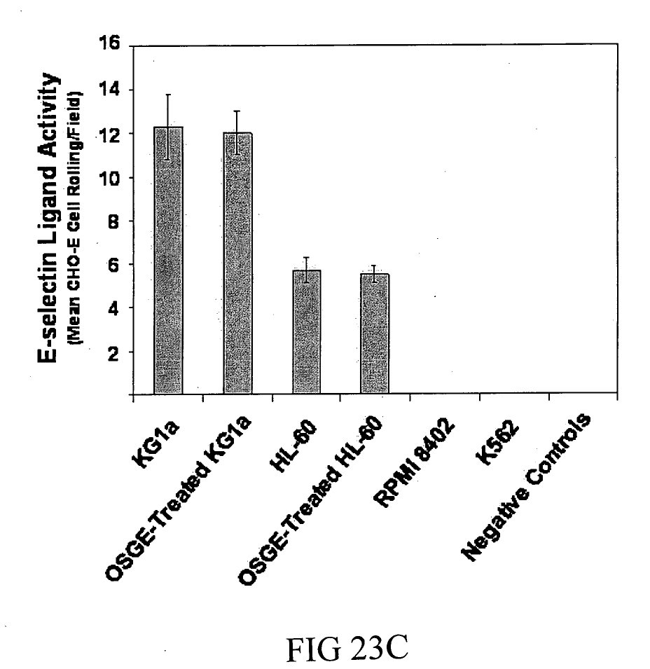

To first identify potential candidate membrane proteins, SDS-PAGE and Western blot analysis of CLA were performed on membrane preparations of the hematopoietic cell lines, KG1a (undifferentiated myelocytic leukemia), HL-60 (promyelocytic leukemia), RPMI 8402 (lymphocytic leukemia) and K562 (erythrocytic leukemia) (16). As illustrated in FIG. 1A, numerous and distinct HECA-452 reactive bands were detected from membrane protein isolated from KG1a cells, but only broad band of ˜140 kDa was detected on HL-60 cells, which most likely corresponded to the monomer species of PSGL-1 (11). There was no HECA-452-reactive membrane protein from RPMI-8402 or K562 cells even though PSGL-1 was detected on Western blots by using anti-PSGL-1 4H10 antibody (17). This suggested that these cells lacked the appropriate HECA-452 binding epitope and, at minimum, the E-selectin binding species of PSGL-1. The CLA/PSGL-1 band was distinguished from other novel HECA-452-reactive bands in KG1a membrane protein by blotting immunoaffinity purified PSGL-1 and non-purified KG1a membrane protein with either HECA-452 or anti-PSGL-1 (FIG. 23B) (18). These data clearly separate the staining pattern of the monomer and dimer forms of KG1a PSGL-1 (220 and ˜140 kDa) from other distinct HECA-452-reactive proteins (FIG. 23B).

To correlate CLA expression to E-selectin ligand activity in these cell lines, the ability of these cells to support E-selectin-mediated adhesive interactions in the parallel-plate flow chamber under shear flow conditions (19) was tested. Using Chinese hamster ovarian cells expressing high levels of E-selectin (CHO-E), there was observed E-selectin-dependent CHO-E cell adhesion to glutaraldehyde-fixed monolayers of KG1A and HL-60 cells at 2.8 dynes/cm² (FIG. 23C). Mock-transfected CHO cells (CHO-mock) displayed no rolling. CHO-E cell rolling was two-fold higher on KG1a cells than on HL-60 cells and was completely inhibited by adding 5 mM EDTA to the assay medium or by preincubating CHO-E cells with anti-E-selectin Abs (10 μg/ml). Also, there as no E-selectin ligand activity on RPMI-8402 and K562 cells, whose membrane proteins, including PSGL-1, were not detectable by HECA-452. Interestingly, E-selectin ligand activities on KG1a and HL-60 cells were not abrogated by pretreatment with O-sialoglycoprotein endopeptidase (OSGE) suggesting the relevant complex selectin binding carbohydrate determinants did not reside sialomucins.

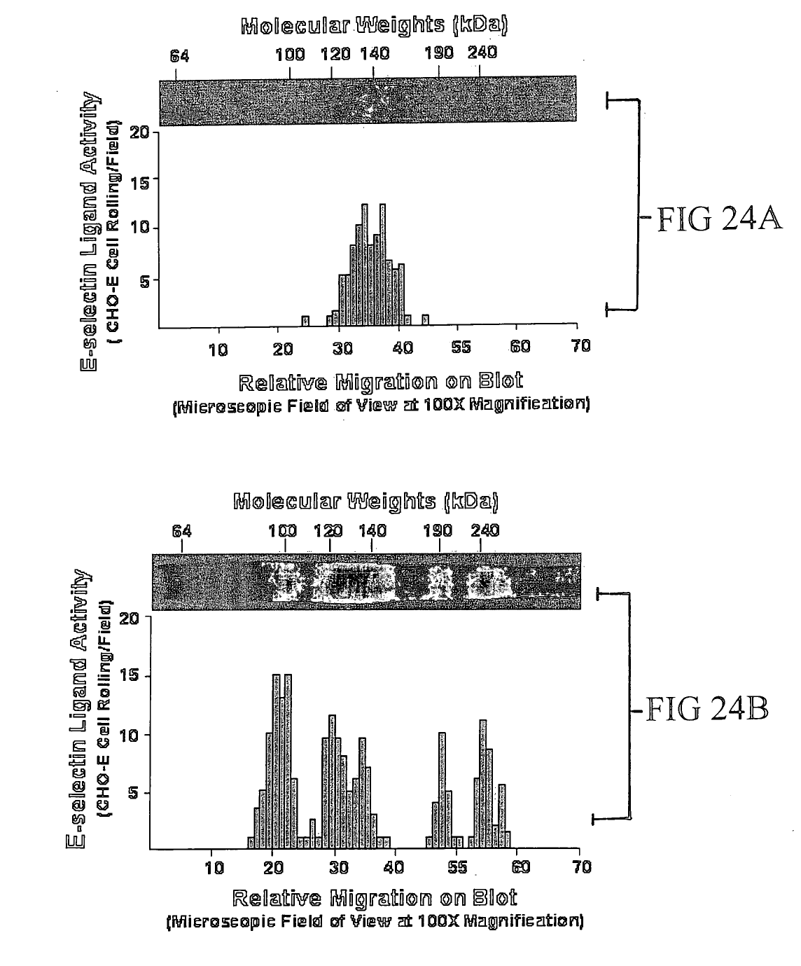

Using a new method for assessing the adhesive interactions under shear flow between selectin-expressing whole cells and proteins immobilized on Western blots (20,21), the ability of CHO-E cells to interact in a E-selectin-dependent manner with Western blots of HECA-452-stained KG1a and HL-60 membrane proteins were analyzed. Membrane proteins separated on 9% SDS-PAGE gel and transferred onto PVDF membrane were stained with HECA-452 and tested for their ability to function as E-selectin ligands. E-selectin-ligand interactions (CHO-E cell tethering and rolling) were rapidly and reproducibly identified on HECA-452 stained bands in real time under defined physiologic flow conditions (3.8 dynes/cm²) by video microscopy (FIGS. 24A and 24B). E-selectin ligand activity occurred, specifically, over the broad HECA-452-stained band (140 kDa) from HL-60 membrane protein, which represented PSGL-1 (FIG. 24A). Of the numerous HECA-stained bands from KG1a membrane protein, E-selectin ligand activity was observed on all major HECA-452-stained bands (98, 120, 130, 190 and 240 kDa) with the greatest frequency of CHO-E cell rolling occurring over the 98 kDa band (FIG. 24B). Where was no evidence of E-selectin-mediated adhesive interactions by CHO-mock cells over HECA-452 stained bands, by adding 5 mM EDTA to the assay medium of CHO-E cells or by pretreating CHO-E cells with anti-E-selectin Abs. E-selectin ligand activity of membrane proteins from both cell lines was directly correlated to HECA-452-staining. However, in all cases, HECA-452-immunostaining did not block ligand activities suggesting that the actual binding determinant for E-selectin was not HECA-452 binding epitopes.

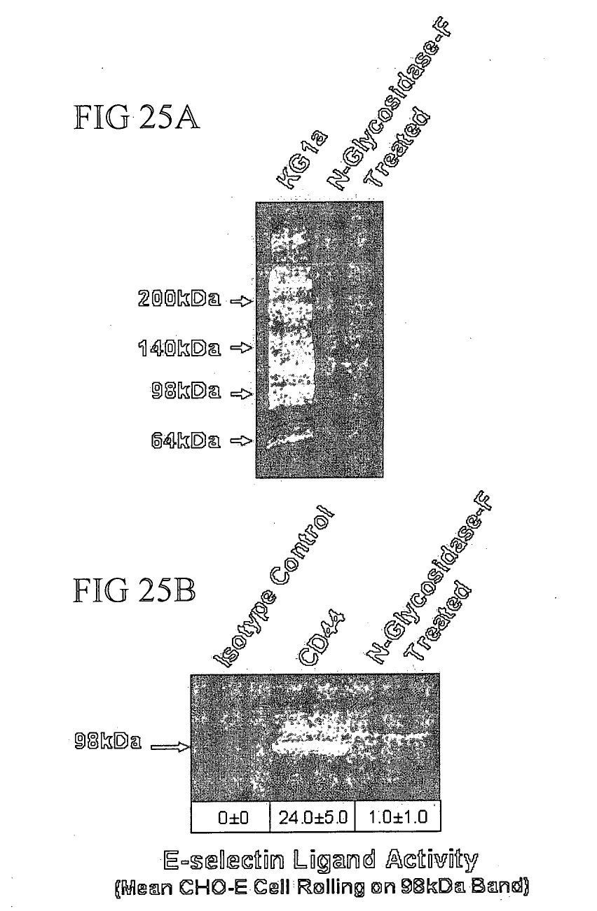

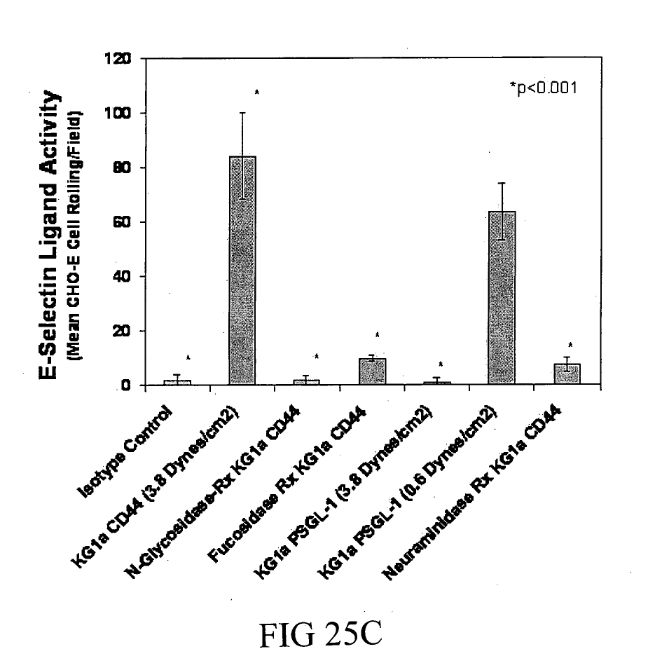

Recent data has shown that a 98 kDa, HECA-452-reactive membrane protein on KG1a cells is a glycoform of the hyaluronic acid receptor, CD44 (22) that functions as a hematopoietic cell L-selectin ligand (HCELL). The structurally active components conferring L-selectin ligand activity on CD44 are the sialylated N-linked carbohydrates (23). Based on these findings and the presence of E-selectin ligand activity at 98 kDa as shown in FIG. 24B, it was suspected that CD44 was functioning as the E-selectin ligand. To analyze whether KG1a CD44 functioned as an E-selectin ligand and was dependent on N-glycans for ligand activity, KG1a membrane proteins were first treated N-glycosidase-F prior to resolution on a 9% SDS-PAGE gel. HECA-452 reactivity of the 98 kDa protein (as well as other proteins at 190 and 240 kDa) was completely dependent on N-glycosylation (FIG. 25A). Furthermore, CD44 was immunoaffinity purified (Hermes-1) from KG1a membrane preparation that was treated with N-glycosidase-F. As shown in FIG. 25B, untreated immunoprecipitated KG1a CD44 was immunodetected with HECA-452 while immunoprecipitated CD44 that was treated with N-glycosidase-F treatment was markedly reduced in HECA-452-reactivity, particularly at 98 kDa isoform, and the ability to function as an E-selectin ligand was completely absent (FIG. 25B), even over the partially stained species detected at 100 kDa.

To further explore the E-selectin ligand activity of CD44, an alternative adhesion assay system was employed wherein Hermes-1-immunoprecipitated CD44 was spotted onto plastic dishes and incorporated into the parallel-plate flow chamber for analysis of E-selectin ligand activity (24). Significantly greater CHO-E cell rolling was observed over KG1a CD44 than on N-glycosidase-F/neuraminidase-treated CD44 or isotype Ab immunoprecipitation, and even KG1a PSGL-1 at 2.8 dynes/cm² (p<0.001) (FIG. 25C). However, at a lower shear force of 0.6 dynes/cm², CHO-E cell rolling on PSGL-1 was significantly elevated, raising the question of differential shear binding capabilities required for temporal and spatial selectin-selectin ligand adhesive interactions, in vivo. These data directly corroborated data generated in the blot adhesion assay system definitively demonstrating that KG1a CD44/HCCLL was also a hematopoietic cell E-selectin ligand (HCEL) and that its sialylated N-glycans were critical to E-selectin binding.

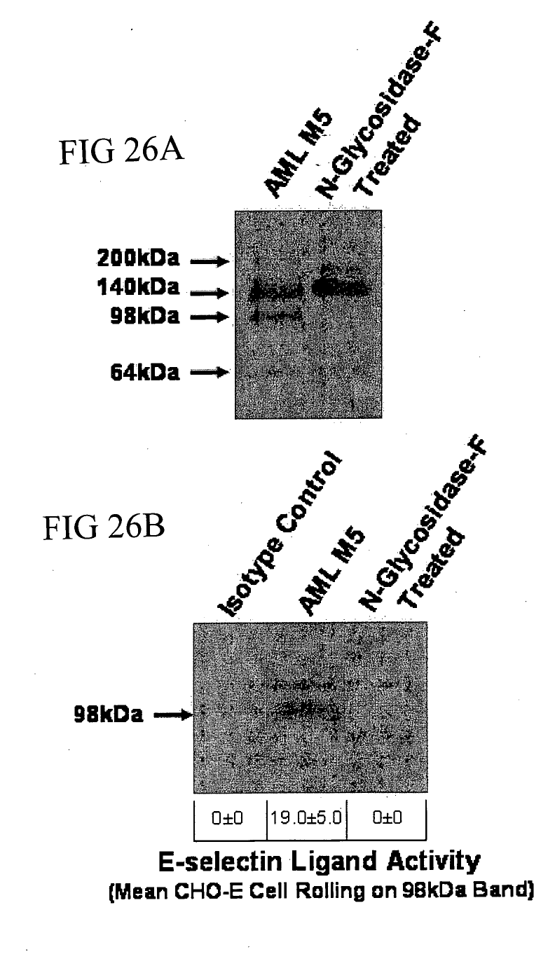

To determine whether E-selectin ligand activity of CD44 was naturally expressed, the expression of E-selectin ligand activity was investigated, specifically of CD44/HCEL, in various cases of acute and chronic leukemias (16). First evaluated was CD44/HCEL activity of an AML subtype M5 from a patient who presented with leukemia cutis. The expression of this phenotype by this leukemia implied that these cells had the propensity to migrate to the skin due to the expression of CLA. Indeed, four major HECA-452 stained bands from membrane proteins of these cells, which were identically prepared and enzymatically-treated as described for KG1a membrane preparations, were detected (FIG. 26A). HECA-452-staining was completely eliminated in the 98kDa following N-glycosidase-F treatment, while the 64 and 140 kDa bands had persistent staining remained intact and the 200 kDa band stained at an apparently reduced molecular weight (FIG. 26A). When immunoprecipitated CD44 from AML M5 membrane protein was treated with N-glycosidase-F, the HECA-452-reactivity as well as the HCEL activity was completely abolished (FIG. 26B). As shown for immunoprecipitated KG1a CD44, the AML M5 CD44 displayed a minor isoform at 100 kDa, detected by HECA-452, with the major band and biologically active protein being the 98 kDa isoform.

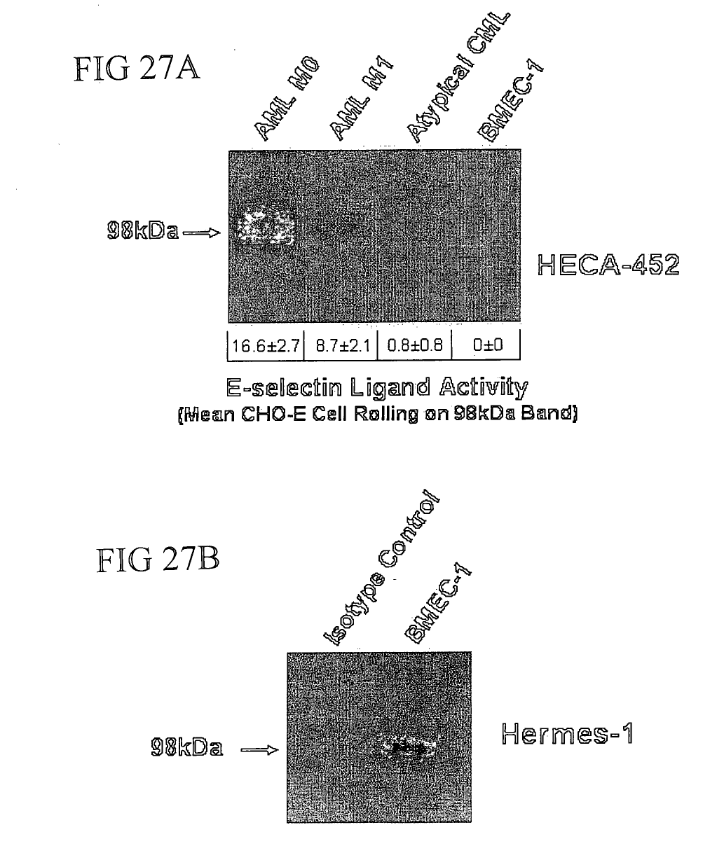

To further determine the E-selectin binding capacity of HECA-452 reactive CD44, the expression HECA-452 reactive, immunoprecipitated CD44 was assessed on an AML M0, an AML M1 and an atypical CML that was negative for brc/abl translocation. The increased expression of the HECA-452-reactive epitopes on immunoprecipitated CD44 was directly correlated with the ability to support E-selectin ligand activity (FIG. 27A). Even though the cell surface expression of CD44 (Hermes-1) on these leukemias was equivalent (>90% positive cell staining by flow cytometry, the ability to interact with E-selectin was absolutely dependent on the presence of HECA-452-reactivity.

Since CD44 appears on multiple cell types including endothelial cells, the investigations were extended to evaluate the ability of CD44 expressed on human bone marrow endothelial cells (BMEC)(16) to engage in E-selectin ligand activity. As illustrated in FIG. 27B, immunoprecipitated BMEC CD44 was detectable by Hermes-1 moAb, however, BMEC CD44 was not HECA-452-reactive and did not possess any E-selectin ligand activity (FIG. 27A). These results confirmed that the expression of CD44, per se, did not confer E-selectin activity. CD44 functions as a scaffold for the appropriate post-translational glycosylations conferring E-selectin binding activity. Indeed, in an earlier study, it was noted that immunoprecipitated CD44 from human tonsils by Hermes-1 moAb did not exhibit E-selectin ligand activity (2). This study, along with these findings, emphasized the importance of appropriate post-translational modification for creating a bioactive E-selectin ligand.

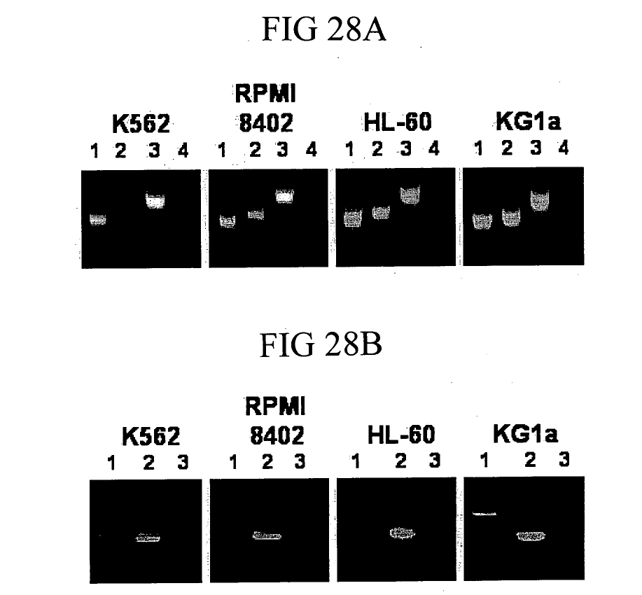

To analyze the role of glycosyltransferases required for HECA-452 antigen decoration, the inherent level of gene expression of α1,3 fucosyltransferases (FucTIV/FucTVII) and α2,3 sialyltransferases (ST2Gal III) was measured by RT-PCR in the human hematopoietic cell lines, K562, RPMI 8402, HL-60 and KG1a. Since the α1,3 fucosylation and α2,3 sialylation of carbohydrate binding determinants by FucTIV and FucTVII and ST3Gal III are critical for E-selectin ligand activity and synthesis of HECA-452 antigen (8, 25-29), it was suggested that these particular enzymes would be differentially expressed in the cell lines that were competent or devoid of E-selectin ligand activity. Moreover, since previous data demonstrated that the expression of CD44 on the leukemia cell lines, KG1a, HL-60 and RPMI-8402, was equivalent (>95% positive cell training by flow cytometry) (30) and E-selectin ligand activity was significantly higher on KG1a than on HL-60 cells or RPMI 8402 cells (FIG. 23C), this strongly suggested that post-translational modifications were crucial for HCEL activity of CD44. FucTIV expression was relatively similar in all cell lines, but the FucTVII expression was highest in HL-60 and KG1a cells (Lane 1, FucTIV, and Lane 2, FucTVII; FIG. 6A). ST3Gal III was expressed at a high level in KG1a cells and was expressed at a very low level in other cell lines (Lane 1; FIG. 28B). This observation highlighted the importance of α2,3 sialylation is a prerequisite for the biosynthesis of sialyl Lewis antigens that are thought to be related to the E-selectin carbohydrate binding determinants.

There have been many descriptions of human glycoconjugates that can serve as E-selectin ligands due to their affinity to human recombinant E-selectin chimeras (31-36), but, with exception of PSGL-1 and myloglycan (37), the identity of these molecules is unknown and their relevance as naturally expressed on the cell surface is not well understood. An E-selectin binding function of CD44 expressed on the human hematopoietic cell line, KG1a, and on freshly isolated de novo leukemias is described herein. This CD44 E-selectin ligand, designated as hematopoietic cell E-selectin ligand or HCEL, is dependent on glycosylations mediated by α1,3 fucosyl- and α2,3 sialyltransferases.

Just as the high expression of HECA-452 epitopes on PSGL-1 (CLA) on skin homing T-cells correlates with the capacity of PSGL-1 to function as an E-selectin ligand and the ability of these cells to traffic to the skin, the expression of HECA-452-reactive CD44 glycoform also correlates with its ability to function as E-selectin ligand. Of note, cutaneous infiltration of leukemia cells from a patient with an AML M5, which was analyzed in this study was observed grossly and by dermatopathologic examination of skin biopsies. The expression of HCEL on leukemia cells, therefore, offers a plausible explanation to the molecular pathophysiology of leukemia cutis. Similarly, since E-selectin is constitutively expressed on BMEC, HCEL this can facilitate normal or leukemic hematopoietic cell re-entry into the bone marrow, having broad implications as a biological surrogate for selection of HCEL+ and CD34+ hematopoietic cell populations for improving engraftment potential of an allogeniec bone marrow transplant and as a biological target for leukemotherapeutic exploitation.

More specifically, FIG. 23 shows the identification of HECA-452-reactive membrane proteins (CLAs) and enhanced E-selectin ligand activity on the human hematopoietic cell line, KG1a. In FIG. 23A there is shown the membrane preparations of human hematopoietic cell lines, KG1a (myeloid) (10 μg), HL-60 (promyeloid) (100 μg), RPMI 8402 (lymphoid) (100 μg) and K562 (erythroid) (100 μg) which were resolved on a reducing 6% SDS-PAGE gel and Western blotted with HECA-452. As depicted in this panel, there were numerous and distinct KG1a membrane proteins stained by HECA-452, while only a single broad band (140 kDa) was detected on HL-60 membrane proteins. In FIG. 23B, to separate HECA-452 stained PSGL-1 from other HECA-452-reactive KG1a membrane proteins, immunoprecipitated PSGL-1 was resolved on a reducing 6% SDS-PAGE gel and Western blotted with either HECA-452 or anti-PSGL-1 antibody (4H10). Lane 1 was loaded with 10 μg of unprecipitated KG1a membrane protein, lane 2 was loaded with immunoprecipitated PSGL-1 from 100 μg of KG1a membrane protein, lane 3 was loaded with 100 μp KG1a membrane protein and lane 4 was loaded with immunoprecipitated PSGL-1 from 100 μg KG1a membrane protein. As noted in this panel, the 220 and 140 kDa forms of PSGL-1 were clearly distinguishable from other HECA-452-reactive KG1a proteins. FIG. 23C shows the results of using the parallel-plate flow assay, which tested the ability of glutaraldehyde-fixed monolayers of hematopoietic cell lines to support E-selectin-mediated tethering and rolling of CHO-E cells at a physiologic shear force of 3.8 dynes/cm². Cellular pre-treatment with O-sialoglycoprotein endopeptidase (OSGE) (60 μg/ml for 1 hour at 37° C.) was performed to assess the contribution of sialomucins to E-selectin ligand activities. KG1a cellular E-selectin ligand activity was two-fold greater than HL-60 ligand activity and was unaffected by OSGE pretreatment. RPMI 8402 and K562 cells did not display any activity. All negative controls consisting of CHO-mock cells, anti-E-selectin Abs (10 μg/ml)-treated CHO-E cells, 5 mM EDTA-containing assay medium or Vibrio cholerae neuraminidase pre-treatment (0.1 U/ml for one hour at 37° C.) of hematopoietic cells, also displayed no activity.

FIG. 24 shows the resolution of E-selectin ligands expressed on the human hematopoietic cell line, KG1a. Western blots of HL-60 and KG1a membrane proteins were separated on a reducing 9% SDS-PAGE gels, blotted onto PVDF membrane, stained with HECA-452 and rendered transparent with 10% glycerol (21) CHO-E cells (1×10⁷/ml) were then perfused over these blots at a defined shear force of 3.8 dynes/cm². FIG. 24A depicts the E-selectin ligand activity as assayed by the number of CHO-E cells rolling in the microscopic field at a particular position on the blot which were identified specifically over the ˜140 kDa HECA-452 stained band from HL-60 membrane protein (100 μg). FIG. 24B shows several HECA-452 stained bands from KG1a membrane protein (10 μg) which correlated with E-selectin ligand activity as evidenced by the inability of weakly stained proteins or unstained regions on the blot to support E-selectin ligand activity. Neither CHO-mock transfectants nor anti-E-selectin treated CHO-E cells did not interact with these proteins, and 5 mM EDTA-containing medium prevented CHO-E rolling.

FIG. 25 shows that HECA-452-reactive CD44 expressed on KG1a cells functions as E-selectin ligand. To assess the sensitivity of HECA-452 epitopes on KG1a membrane proteins to N-glycosidase-F treatment (8 U/ml for 18 hours at 37° C.), membrane proteins were treated (10 μg) prior to separation on a reducing 9% SDS-PAGE gel and immunostained with HECA-452 (FIG. 25A). As shown, HECA-452 reactivity persisted at 120 and 140 kDa, but the 240, 190, 98 and 64 kDa bands were undetectable. To analyze whether the HECA-452 reactive band at 98 kDa was CD44, CD44 (anti-CD44, rat IgG, Hermess-1) was immunoprecipitated from KG1a membrane protein (50 μg) (FIG. 25B). Immunoprecipitated protein was then resolved on a reducing 9% SDS-PAGE gel, transferred to PVDF membrane and immunostained with HECA-452. E-selectin-mediated CHO-E cell rolling was observed at 3.8 dynes/cm² over the 98 kDa isoform of CD44, but no activity was observed over the 120 and ˜140 kDa bands representing isoforms of CD44 (38). To address the presence and contribution of N-glycans displayed on KG1a CD44 to E-selectin ligand activity, denatured KG1a membrane proteins (50 μg) were treated with N-glycosidase-F prior to immunoprecipitation and resolution by HECA-452 blotting. As illustrated, N-glycosidase-F treatment cleaved the HECA-452-reactive epitope(s) on the 98 kDa isoform of CD44 and completely eliminated CHO-E cell rolling. To account for the expected shift in molecular weight due to the de-N-glycosylation following N-glycosidase treatment, the entire 50-200 kDa molecular weight range was tested for E-selectin ligand activity, and did not possess any activity. There was no detectable immunoprecipitate with isotype control rat IgG confirming the specificity of Hermes-1 immunoaffinity precipitation of CD44. Immunoprecipitated CD44 (Hermes-1 moAb) or PSGL-1 (4H10 moAb) was spotted onto plastic dishes, fixed in 3% glutaraldehyde, treated with 0.2M lysine, blocked in 100% FBS and incorporated into the parallel-plate flow chamber for analysis of E-selectin ligand activity (23) (FIG. 25C). E-selectin-mediated CHO-E cell rolling was observed at 3.8 dynes/cm² on KG1a CD44 and no binding was observed on KG1a PSGL-1 at an equivalent shear force. However, PSGL-1-supported CHO-E rolling did occur at a lower shear force of 0.6 dynes/cm². N-glycosidase-F-treated K1a CD44 and Vibrio cholerae neuraminidase treatment markedly ablated CHO-E cell rolling (Paired t-test; p<0.001). No CHO-E cell rolling was observed on rat IgG or mouse IgG affinity purified KG1a protein or on Abs alone.

FIG. 26 shows that HECA-452-reactive CD44 from freshly isolated acute myelogenous leukemia cells of subtype M5 (AML M5) functions as an E-selectin ligand. AML M5 cells were isolated from the peripheral blood of a patient that presented with cutaneous leukemic infiltrate, and membrane proteins (50 μg) were prepared and immunostained with HECA-452 as previously described (16). Membrane proteins were also treated with N-glycosidase-F (8 U/ml for 18 hours at 37° C.) to assess the dependence of N-glycosylation for HECA-452 staining (16). Several membrane proteins were detected by HECA-452, but only the 98 kDa band was eliminated by N-glycosidase-F pretreatment, which coincided with the sensitivity of HECA-452-epitopes on he N-glycans of KG1a CD44 (FIG. 26A). To analyze the expression of HECA-452-reactive epitopes on CD44, CD44 was immunoaffinity purified from AML M5 membrane protein (50 μg), separated on a 9% SDS-PAGE gel and blotted with HECA-452 (FIG. 26B). The 98 kDa CD44 isoform supported E-selectin-mediated CHO-E cell rolling, while CHO-mock transfectants, anti-E-selectin-mediated CHO-E cell rolling or CHO-E cells containing 5 mM EDTA in their medium did not interact with the CD44, 98 kDa band. Detection of the 98 kDa isoform of CD44 was eliminated due to N-glycosidase-F treatment, and E-selectin ligand activity (CHO-E cell rolling) was absent over a 50-200 kDa molecular eight range, indicating that the absence of HECA-452 reactive epitopes on CD44 correlated with the removal of the critical binding determinants and not a consequence of a shift in molecular weight due to de-N-glycosylation.

FIG. 27 shows the differential expression and E-selectin ligand activity of HECA-452-reactive CD44 from freshly isolated leukemias and human bone marrow endothelial cells. Immunoprecipitated CD44 from membrane preparations (50 μg) of leukemia cells isolated from patients with an AML M0, AML M1 or atypical CML (brc/abl), or immunoprecipitated CD44 from membrane proteins (100 μg) of human bone marrow endothelial cells (BMEC) was prepared, separated on a 6% SDS-PAGE gel and immunostained with HECA-452 or Hermes-1 and evaluated for E-selectin ligand activity. As noted the panel (FIG. 27A), the most HECA-452-reactive CD44 molecule (AML M0) elicited the highest E-selectin ligand activity. In Panel (FIG. 27B), immunoprecipitated CD44 was detectable by anti-CD44 Hermes-1 moAb, but the lack of HECA-452-reactive epitopes on BMEC CD44 correlated with its inability to function as an E-selectin ligand.

FIG. 28 shows the expression of α1,3 fucosyltransferase IV (FucTIV), α1,3 fucosyltransferase VII (FucTVIi) and α2,3 sialyltransferase (ST3Gal III), on hematopoietic progenitor cell lines. To assess the inherent capability of KG1a, HL-60, RPMI 8404 and K562 cells to synthesize sialyl Lewis X epitopes, RT-PCR expression analysis of FucTIV was performed, FucTVII and ST3Gal III (39). FucTIV (Lane 1) expression was relatively similar in all cell lines; however, HL-60 and KG1a cells possession the highest levels of FucTVII (Lane 2) (FIG. 28A). Lanes 3 (β-actin) and 4 (ddH₂0) served as positive and negative controls, respectively. Interestingly, ST3Gal III (Lane 1) was elevated only in the KG1a cells in comparison with the other cell lines (FIG. 28B). Lane 2 (β-actin) and Lane 3 (ddH₂O) were the positive and negative controls, respectively.

Throughout this application various publications or patents are referenced by citation or number. Full citations for the publications referenced are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE 1
Expression of Surface Molecules on Cell Lines Utilized in the Lymphocyte Adherence Assay.

| | LYMPHOCYTE | | RELATIVE EXPRESSION OF MEMBRANE PROTEINS* | | | | | |
|---|---|---|---|---|---|---|---|---|
| CELL LINE | ADHERENCE | LINEAGE | CD34 | LFA-1 | VLA-4 | CD44 | Sialyl Le* | CD43 |
| KG1a | YES | Myeloid | ++++ | ++++ | ++++ | ++++ | ++++ | ++++ |
| RPMI 8402 | NO | Lymphoid | ++++ | − | ++++ | +++ | − | ++++ |
| HL60 | NO | Myeloid | − | − | ++++ | + | − | ++++ |
| Nalm 16 | NO | Lymphoid | − | − | ++++ | − | +++ | ++++ |
| K562 | NO | Erythroid | − | − | − | − | ++ | +++ |
| Raji | NO | Lymphoid | − | +++ | ++++ | − | − | + |

*Percentage of positive cells as determined by flow cytometric analysis.
− = 0-5% positive
+ = 6-35% postive
++ = 36-65% positive
+++ = 66-95% positive
++++ = >96% positive

TABLE 2
Lymphocyte Adherence to KG1a

| | Mean (SEEM) of Binding | |
|---|---|---|
| | (% of Untreated Control) | |
| LYMPHOCYTE TREATMENT | | |
| EDTA | 0.3 | (0.3) |
| Mannose-6-P | 5.7 | (1.0) |
| Fucoidin | 1.4 | (0.4) |
| PPME | 5.4 | (0.5) |
| LAM1-3 mAb | 1.9 | (0.4) |
| Anti CD45 mAb | 98.7 | (6.3) |
| IgG₁ Control mAb | 115.1 | (9.0) |
| PMA | 1.1 | (0.3) |
| KG1A TREATMENT: | | |
| Anti CD34 mABs^{†} | 116.2 | (7.7) |
| Anti CD45 mAb | 98.0 | (3.6) |
| IgG₁ Control mAb | 101.8 | (8.5) |
| CD34-Positive Sort | 102.8 | (3.5) |
| CD34-Negative Sort | 104.1 | (4.2) |
| Neuraminidase | 3.1 | (0.7) |
| Neuraminidase Buffer Control | 100.5 | (6.7) |
| O-Sialoglycoprotein Endopeptidase | 98.4 | (2.3) |
| Bromelain | 3.8 | (0.4) |
| Chymotrypsin | 6.7 | (0.7) |
| Chymotrypsin, PMSF, Chymostatin | 94.0 | (3.8) |
| Combination of HPCA-1, HPCA-2, | | |
| 12.8 and QBEND10 mAbs. | | |

TABLE 3
Lymphocyte Adherence to KG1a

| | Mean (SEM) of Binding | |
|---|---|---|
| KG1a Treatment | (% of untreated control)** | |
| Neuraminidase (t = 0, no culture) | 0.02 | (0.1) |
| Neuraminidase Buffer Control (no culture) | 98.5 | (7.5) |
| Neuraminidase, 24 hr culture | 101.3 | (6.5) |
| Neuraminidase, 24 hr culture with Chlorate | 107.1 | (6.2) |
| (10 mM) | | |
| Neuraminidase, 20 hr culture with | 0.4 | (0.1) |
| Tunicamycin (15 ug/ml) | | |
| Neuraminidase, 20 hr culture with | 105.2 | (7.5) |
| Tunicamycin Buffer alone | | |
| 20 hr culture with Tunicamycin (15 ug/ml) | 106.2 | (7.2) |
| (no neuraminidase pretreatment) | | |
| Neuraminidase, 20 hr culture with | 0.5 | (0.2) |
| Cycloheximide (1.25 ug/ml) | | |
| 20 hr culture with Cycloheximide | 91.8 | (5.8) |
| (1.25 ug/ml) | | |
| (no neuraminidase pretreatment) | | |

*Following neuraminidase treatment, all cells were washed prior to culturing as indicated. Experimental details are described in text.
**Number of lymphocytes adherent to confluent area of KG1a were counted by light microscopy using an ocular grid under 250× magnification (quantified a minimum of 2 fields/slide, 2 slides/experiment, 3 separate experiments). Results are presented as percent binding compared with corresponding untreated control KG1a cyto-spin preparations.

TABLE 1
L-selectin Ligand Activity of KG1a Cells Treated with
Anti-PSGL-1 Antibodies or P-selectin-IgG Chimera

| Blocking Treatments | % Control Binding* (SEM) |
|---|---|
| Anti-PSGL-1 monoclonal antibodies | 103.0 (7.0) |
| (20 ig/ml of each in a cocktail) | |
| P-selectin-IgG chimera, 50 ig/ml | 97.6 (7.4) |

*Number of lymphocytes adherent to confluent area of KG1a were counted by light microscopy using an ocular grid under 100× magnification (quantified a minimum of 2 fields/slide, 3 slides/experiment, 3 separate experiments). Results are presented as percent binding compared with corresponding untreated control KG1a cytospin preparations.

TABLE 2
L-selectin ligand Activity of KG1a Cells Following
Protease or Phospholipase C Treatment

| Enzymatic Treatments | % Control Binding* (SEM) | |
|---|---|---|
| O-Sialoglycoprotease | 98.4 | (2.3) |
| Mocarhagin | 98.9 | (6.3) |
| Bromelain | 3.8 | (0.4)** |
| Chymotrypsin | 6.7 | (0.7)** |
| Pronase | 9.4 | (1.3)** |
| Porcine Pancreas Elastase | 47.3 | (17.6)** |
| Human Neutrophil Elastase | 25.4 | (13.5)** |
| PI-PLC | 98.1 | (2.0) |

*Number of lymphocytes adherent to confluent area of KG1a were counted by light microscopy using an ocular grid under 100× magnification (quantified a minimum of 2 fields/slide, 3 slides/experiment, 3 separate experiments). Results are presented as percent binding compared with corresponding untreated control KG1a cytospin preparations.
**Statistically significant (Paired t-test; p < 0.01) difference compared with enzyme buffer alone group.

TABLE 3
Re-Expression of L-selectin ligand Activity Following
Neuraminidase or Protease Treatment of KG1a Cells
is Dependent on de novo Protein Synthesis

| | Mean (SEM) | |
|---|---|---|
| KG1a Treatment* | % Control Binding** | |
| Neuraminidase (t = 0, no culture) | 0.2 | (0.1)⁺ |
| Neuraminidase Buffer Control (no culture) | 98.5 | (7.5) |
| Neuraminidase, 24 hour culture | 101.3 | (6.5) |
| Bromelain (t = 0, no culture) | 3.8 | (0.4)⁺⁺ |
| Bromelain, 20 hour culture | 95.2 | (7.1) |
| Neuraminidase, 20 hour culture with | 0.5 | (0.2)⁺ |
| Cycloheximide (1.25 μg/ml) | | |
| Bromelain, 20 hour culture with Cycloheximide | 0.6 | (0.3)⁺⁺ |
| (1.25 μg/ml) | | |
| 20 hour culture with Cycloheximide (1.25 μg/ml) | 91.8 | (5.8) |
| (no neuraminidase or bromelain digestion) | | |

*Following neuraminidase or bromelain treatment, all cells were washed prior to culturing as indicated.
**Number of lymphocytes adherent to confluent area of KG1a were counted by light microscopy using an ocular grid under 100× magnification (quantified a minimum of 2 fields/slide, 3 slides/experiment, 3 separate experiments). Results are presented as percent binding compared with corresponding untreated control KG1a cyto-spin preparations.
^{+,++}Statistically significant (p < 0.01, by paired t-test) difference compared to undigested, neuraminidase buffer-incubated (⁺) or RPMI 1640-incubated (⁺⁺) cells. All other values are not statistically different (p > 0.05).

TABLE 4
L-selectin Ligand Activity of KG1a cells Treated
With Inhibitors of N-linked Glycosylation

| KG1a Treatment¹ | % Control Adhesion (SEM)² | |
|---|---|---|
| Neuraminidase (1 h) | 1.0 | (0.7)* |
| Neuraminidase (1 h then 24 h recovery) | 99.3 | (1.3) |
| Tunicamycin alone | 96.5 | (7.4) |
| Deoxymannojirimycin Alone | 107.5 | (6.7) |
| Swainsonine alone | 95.5 | (10.3) |
| Neuraminidase + Tunicamycin | 1.0 | (0.7)* |
| Neuraminidase + Deoxymannojirimycin | 22.5 | (0.4)* |
| Neuraminidase + Swainsonine | 26.6 | (2.5)* |

¹KG1a cells were cultured in the presence of tunicamycin (15 μg/ml), deoxymannojirimycin (0.4 mg/ml) or swainsonine (40 μg/ml) for 24 h after a 1 h (pre-culture) treatment with V. Cholera neuraminidase (0.1 U/ml). Cytospin preparations of these cells were then subjected to Stamper-Woodruff assay as detailed in the text. L-selectin-specific lymphocyte binding to cytospins was quantified by light microscopy.
²Values represent mean percent lymphocyte adherence (standard error of the mean) compared with untreated control group from three independent experiments. Adherent lymphocytes were counted from four fields on each cytospin in triplicate slides using an optical grid under 100× magnification.
*Statistical significance (paired t-test; p < 0.01) compared with neuraminidase (1 h then 24 h recovery) group.

TABLE 5
L-selectin Ligand Activity of KG1a
cells Treated With N-glycosidases

| N-Glycosidase Treatment of | | |
|---|---|---|
| KG1a Membrane Preparation¹ | % Control Adhesion (SEM)² | |
| Endo H | 105.5 | (2.5) |
| Endo F2 | 66.9 | (0.1)* |
| PNG-F | 16.0 | (0.7)** |

¹KG1a membrane preparations (0.2 mg/ml) were incubated with either Endo H (50 mU/ml), Endo F2 (4 mU/ml), PNG-F (8 U/ml) or respective buffers (controls) for 24 h at 37° C., then spotted onto glass slides (1 μg/spot) and subjected to Stamper-Woodruff assays as detailed in the text. L-selectin-specific lymphocyte binding to spotted samples was quantified by light microscopy.
²Values represent mean percent adherence (standard error of the mean) compared with buffer control groups from three independent experiments. Adherent lymphocytes were counted from four fields on each spot in triplicate slides using an optical grid under 100× magnification.
*Statistical significance (Paired t-test; p = 0.03) compared with buffer control group.
**Statistical significance (Paired t-test; p < 0.01) compared with buffer control group.

### REFERENCES

Abdullah et al., ²A neutral glycoprotease of Pasteurella haemolytica A1 specifically cleaves O-sialoglycoproteins² Infect Immun., 60:56, (1992).
Aruffo et al., ²CD62/P-selectin recognition of myeloid and tumor cell sulfatides² Cell, 67:35-44 (1991).
Baeuerle and Huttner, ²Chlorate-a potent inhibitor of protein sulfation in intact cells² Biochemical and Biophysical Res. Comm., 141:870-877 (1986).
Baumhueter et al., ²Binding of L-selectin to the vascular sialomucin CD34² Science 262:436, (1993).
Berg et al., ²L-selectin mediated lymphocyte rolling on MAd-CAM-1² Nature, 366:695-698 (1993).
Beschorner et al., ²Localization of hematopoietic progenitor cells in tissue with the anti-My-10 monoclonal antibody² Am J Pathol 119:1, (1985).
Bevilacqua and Nelson, ²Selectins² J Clin Invest., 91:379, (1993).
Bollman et al., ²Techniques for the collection of lymph from the liver, small intestine, or thoracic duct of the rat² J Lab Clin Med., 33:1349, (1948).
Carlos and Harlan, ²Leukocyte-Endothelial adhesion molecules² Blood, 84(7):2068, (1994).
Chin et al., ²Lymphocyte-homing receptors and preferential migration pathways² Proc Soc Exp Biol Med., 196:374, (1991).
Civin et al., ²Antigenic analysis of hematopoiesis III. A hematopoietic progenitor cell surface antigen defined by a monoclonal antibody raised against KG-1a cells² J Immunol., 133:157, (1984).
Cowing and Chapdelaine, ²T cells discriminate between la antigens expressed on allogeneic accessory cells and B cells: a potential function for carbohydrate side chains on la molecules² Proc Natl Acad Sci USA, 80:6000, (1983).
Cullen et al., “Differential glycosylation of murine B cell and spleen adherent cell la antigens” J Immunol., 127:1478, (1981).
Cytospin®3 Cell Preparation System Operator Guide, Shandon Scientific Limited.
Dercksen et al., ²Expression of adhesion molecules on CD34+ cells: CD34+ L-selectin+ cells predict a rapid platelet recovery after peripheral blood stem cell transplantation² Blood, 85:3313-3319 (1995).
Dowbenko et al., ²Structure and chromosomal localization of the murine gene encoding GlyCAM-1² J. Clin. Invest., 92:952-960 (1993).
Gallatin, et al., ²A cell-surface molecule involved in organ-specific homing of lymphocytes² Nature, 304:30-34 (1983).
Gatenby et al., ²Dissection of immunoregulatory subpopulations of T lymphocytes within the helper and suppressor sublineages in man² J. Immunol., 129:1997, (1982).
Gowans and Knight, ²The route of re-circulation of lymphocytes in the rat² Proc Roy Soc Lond B., 159:257, (1964).
Green et al., ²Further studies of the binding specificity of the leukocyte adhesion molecule, L-selectin, towards sulphated oligosaccharides-suggestion of a link between the selectin- and the integrin-mediated lymphocyte adhesion systems² Glycobiology, 5:29-38 (1995).
Green et al., ²High affinity binding of the leukocyte adhesion molecule L-selectin to 3′-sulphated-Le^{x} and -Le^{x} oligosaccharides and the predominance of sulphate in this interaction demonstrated by binding studies with a series of lipid-linked oligosaccharides² Biochem. Biphys. Res. Comm., 188:244-251 (1992).
Griffin et al., ²Granulocyte-macrophage colony-stimulating factor and other cytokines regulate surface expression of the leukocyte adhesion molecule-1 on human neutrophils, monocytes, and their precursors² J Immunol., 145:576, (1990).
Hemmerich, et al., ²Sulfation-dependent recognition of high endothelial venules (HEV)-ligands by L-selectin and MECA79, an adhesion blocking monoclonal antibody² J. Exp. Med., 180:2210-2226 (1994).
Hemmerich and Rosen, ²6′-sulfated sialyl lewis x is a major capping group of GlyCAM-1² Biochemistry, 33:4830-4835 (1994b).
Huff et al., ²Modulation of the biologic activities of IgE-binding factors. III. switching of a T cell hybrid clone from the formation of IgE-suppressive factor to the formation of IgE-potentiating factor2 J Immunol., 131:1090, (1983).
Imai et al., ²Sulphation requirement for GlyCAM-1, an endothelial ligand for L-selectin² Nature 361:555, (1993).
Imai et al., ²Identification of a carbohydrate-based endothelial ligand for a lymphocyte homing receptor² J Cell Biol., 113:1213, (1991).
Kobayashi et al., ²Expression of adhesion molecules on human hematopoietic progenitor cells at different maturational stages² Stem Cells, 12:316, (1994).
Koeffler et al., ²An undifferentiated variant derived from the human acute myelogenous leukemia cell line (KG-1)² Blood 56:265, (1980).
Koenig et al., Blood, 84:599 (1994).
Krause et al., ²Two forms of CD34 protein are expressed in human KMT2 and KG1a cells² Blood, 82:110a, (1993) (abstr).
Lasky et al., ²An endothelial ligand for L-selectin is a novel mucin-like molecule² Cell, 69:927, (1992).
Lewinsohn et al., ²Leukocyte-endothelial cell recognition: evidence of a common molecular mechanism shared by neutrophils, lymphocytes, and other leukocytes² J Immunol., 138:4313, (1987).
Majdic et al., ²Signaling and induction of enhanced cytoadhesiveness via the hematopoietic progenitor cell surface molecule CD34² Blood, 83:1226, (1994).
Marchesi and Gowans, ²The migration of lymphocytes through the endothelium of venules in lymph nodes: an electron microscope study² Proc Rov Soc Lond B, 159:283, (1964).
Niehrs et al., Chemico-Bio. Interactions, 92:257-271 (1992).
Ord et al., ²Structure of the gene encoding the human leukocyte adhesion molecule-1 (TQ1, Leu-8) of lymphocytes and neutrophils² J Biol Chem., 265:7760, (1990).
Oxley and Sackstein, ²L-selectin-CD34 Interactions mediate functional binding of lymphocytes to hematopoietic progenitor cells² Clin Res. Abs. 42(2):235A, April (1994).
Oxley and Sackstein, ²Detection of an L-selectin ligand on a hematopoietic progenitor cell line² Blood, 84:3299-3306 (1994b)
Pouyani and Seed, ²PSGL-1 recognition of P-selectin is controlled by a tyrosine sulfation consensus at the PSGL-1 amino terminus² Cell, 83:333-343 (1995).
Puri et al.,²Sialomucin CD34 is the major L-selectin ligand in human tonsil high endothelial venules² J. Cell Bio., 131:261-270 (1995).
Ramakrishnan, ²Current status of antibody-toxin conjugates for tumor therapy² Targeted Diagn Ther., 3:189, (1990).
Rasmussen et al., ²Lymphocyte recognition of lymph node high endothelium. VII. Cell surface proteins involved in adhesion defined by monoclonal anti-HEBFLN (A.11) antibody² J. Immunol., 135:19-24 (1985).
Reinherz et al., ²Heterogeneity of human T4⁺ inducer T cells defined by a monoclonal antibody that delineates two functional subpopulations² J Immunol., 128:463,1982.
Rosen et al., ²Involvement of sialic acid on endothelial cells in organ-specific lymphocyte recirculation² Science, 228:1005, (1985).
Rosen, ²Cell surface lectins in the immune system² Sem Imm., 5:237, (1993).
Rosen, ²Localization of ligands for L-selectin in mouse peripheral lymph node high endothelial cells by colloidal gold conjugates² Blood, 84:3766-3775 (1994).
Sackstein, et al., ²Evidence of post-transcriptional regulation of L-selectin gene expression in rat lymphoid cells² Immunol., 85:198-204 (1995).
Sackstein, ²Physiological migration of lymphocytes to lymph nodes following bone marrow transplantation: Role in immune recovery² Seminars in Oncology 20(5), Suppl 6:34, (1993).
Sackstein et al., ²Lymphocyte adhesion to psoriatic dermal endothelium is mediated by a tissue-specific receptor/ligand interaction² J Invest Dermatol., 91:423, (1988).
Sako et al., ²A sulfated peptide segment at the amino terminus of PSGL-1 is critical for P-selectin binding² Cell, 83:323-331 (1995).
Selden, ²Transfection using DEAE-dextran², Ausubel F M, Brent R, Kingston R E, Moor D D, Seidman J G, Smith J A, Struhl K (eds) in Current Protocols in Molecular Biology, Vol 2. New York N.Y., John Wiley & Sons, p. 9.2.1. (1992).
Sharon and Lis, ²Carbohydrates in cell recognition² Sci. Amer., 82-89, Jan.(1993).
Shaw et al., ²Two antigen-independent adhesion pathways used by human cytotoxic T-cell clones² Nature, 323:262, (1986).
Shimazu and Shaw, “Mucins in the mainstream” Nature 366:630-631 (1993).
Spertini et al., ²Leukocyte adhesion molecule-1 (LAM-1, L-selectin) interacts with an inducible endothelial cell ligand to support leukocyte adhesion² J Immunol., 147:222565, (1991).
Spertini et al., ²Function and evolutionary conservation of distinct epitopes on the leukocyte adhesion molecule-1 (TQ1, Leu-i) that regulate leukocyte migration² J Immunol., 147:942 (1991).
Springer et al., ²The lymphocyte function-associated LFA-1, CD2, and LFA-3 molecules: Cell adhesion receptors of the immune system² Ann Rev Immunol., 5:223-252 (1987).
Stamper and Woodruff, “Lymphocyte homing into lymph nodes: in vitro demonstration of the selective affinity of recirculating lymphocytes for high-endothelial venules” J Exp Med., 144:828-833 (1976).
Stoolman et al., “Phosphomannosyl receptors may participate in the adhesive interaction between lymphocytes and high endothelial venules” J Cell Biol., 99:1111535 (1984).
Stoolman and Rosen, “Possible role for cell-surface carbohydrate-binding molecules in lymphocyte recirculation” J Cell Biol., 96:722 (1983).
Streeter et al., “A tissue-specific endothelial cell molecule involved in lymphocyte homing” Nature, 331:41 (1988).
Sutherland et al.,“Differential sensitivity of CD34 epitopes to cleavage by Pasteurella haemolytica glycoprotease: Implications for purification of CD34-positive progenitor cells” Exp Hematol,. 20:590 (1992).
Suzuki et al., ²Sulfated glycolipids are ligands for a lymphocyte homing receptor, L-selectin (LECAM-1), binding epitope in sulfated sugared chain² Biochem. Biophys. Res. Comm., 190:426-434 (1993).
Tedder et al., “Expression of the human leukocyte adhesion molecule, LAM1, identity with the TQ1 and Leu-8 differentiation antigens” J Immunol., 144:532-540 (1990).
Terstappen et al., “Induction of human hematopoietic stem cell outgrowth by L-selectin ligation” Blood 82:109a (1993) (abstr) Terstappen et al., “Flow cytometric assessment of human T-cell differentiation in thymus and bone marrow” Blood 79:666 (1992).
True et al., “Requirement for sialic acid on the endothelial ligand of a lymphocyte homing receptor” J Cell Biol., 111:2757 (1990).
Watson et al., “A homing receptor-IgG chimera as a probe for adhesive ligands of lymph node high endothelial venules” J Cell Biol., 110:2221 (1990).
Wilson and Rider, ²Evidence that Leukosialin, CD43, is intensely sulfated in the murine T lymphoma line RDM-4² J. Immunol., 148:1777-1783 (1992).
Woodruff et al., “An in vitro model of lymphocyte homing II. Membrane and cytoplasmic events involved in lymphocyte adherence to specialized high-endothelial venules of lymph nodes” J Immunol., 119:1603 (1977).
Woodruff et al., “Specific cell-adhesion mechanisms determining migration pathways of recirculating lymphocytes” Ann Rev Immunol., 5:201-222 (1987).
Yamashita et al., “Organ-specific difference in the sugar chains of gamma-glutamyltranspeptidase. Arch Biochem Biophys., 225:993 (1983).
Yamashita et al., “The structures of the carbohydrate moieties of mouse kidney gamma-glutamyltranspeptidase: occurrence of X-antigenic determinants and bisecting N-acetylglucosamine residues” Arch Biochem Biophys., 240:573 (1985).
Yednock et al., ²Phosphomannosyl-derived beads detect a receptor involved in lymphocyte homing² J. Cell Biol., 104:713-723 (1987).
Carlos T M, Harlan J M. Leukocyte-Endothelial Adhesion Molecules. Blood. 1994;84:2068-2101.
Sackstein R. Expression of an L-selectin ligand on hematopoietic progenitor cells. Acta Haematol. 1997;97:22-28.
Mazo I B, Gutierrez-Ramos J-C, Frenette P S, Hynes R O, Wagner D D, von Andrian U H. Hematopoietic progenitor cells rolling in bone marrow microvessels: Parallel contributions by endothelial selectins and vascular cell adhesion molecule 1. J. Exp. Med. 1998;188:465-474.
Simmons P, Zannettino A, Gronthos S, Leavesley D. Potential adhesion mechanisms for localization of hematopoietic progenitors to bone marrow stroma. Leuk. and Lymphoma. 1994; 12:353-363.
Verfaille C M. Adhesion receptors as regulators of the hematopoietic process. Blood. 1998;92:2609-2612.
Lévesque J-P, Zannettino A C W, Pudney M, et al. PSGL-1 -mediated adhesion of human hematopoietic progenitors to P-selectin results in suppression of hematopoiesis. Immunity. 1999;11:369-378.
Koenig J M, Baron S, Luo D, Benson N A, Deisseroth A B. L-selectin expression enhances clonogenesis of CD34+ cord blood progenitors. Pediatr. Res. 1999;45:867-870.
Dercksen M W, Gerritsen W R, Rodenhuis S, et al. Expression of adhesion molecules on CD34+ L-selectin+ cells predicts a rapid platelet recovery after peripheral blood stem cell transplantation. Blood. 1995;85:3313-3319.
Watanabe T, Dave B, Heimann D G, Jackson J D, Kessinger A, Talmadge J E. Cell adhesion molecule expression on CD34+ cells in grafts and time to myeloid and platelet recovery after autologous stem cell transplantation. Exp. Hematol. 1998;26:10-18.
Schweitzer K M, Dräger A M, van der Valk P, et al. Constitutive expression of E-selectin and VCAM-1 on endothelial cells of the hematopoietic tissues. Am. J. Pathol. 1996;148:165-175.
Frenette P S, Subbarao S, Mazo I B, von Andrian U H, Wagner D D. Endothelial selectins and vascular cell adhesion molecule-1 promote hematopoietic progenitor homing to the bone marrow. Proc. Natl. Acad. Sci. USA. 1998;95:14423-14428.
Terstappen L W W M, Huang S, Bui N, Picker L J. Induction of human hematopoietic stem cell outgrowth by L-selectin ligation [abstract]. Blood. 1993;82:109a.
Gunji Y, Nakamura M, Hayakawa T, et al. Expression and function of adhesion molecules on human hematopoietic stem cells: CD34+ LFA-1-cells are more primitive than CD34+ LFA-1+ cells. Blood. 1992;80:429-436.
Bertozzi C R. Cracking the carbohydrate code for selectin recognition. Chemistry & Biology. 1995;2:703-708.
Varki A. Selectin ligands. Proc. Natl. Acad. Sci. USA. 1994;91:7390-7397.
Imai Y, Singer M S, Fennie C, Lasky L A, Rosen S D. Identification of a carbohydrate-based endothelial ligand for a lymphocyte homing receptor. J. Cell Biol. 1991;113:1213-1221.
Lasky L A, Singer M S, Dowbenko D, et al. An endothelial ligand for L-selectin is a novel mucin-like molecule. Cell. 1992;69:927-938.
Hemmerich S, Butcher E, Rosen S D. Sulfation-dependent recognition of high endothelial venules (HEV)-ligands by L-selectin and MECA 79, an adhesion-blocking monoclonal antibody. J. Exp. Med. 1994;180:2219-2226.
Sassetti C, Tangemann K, Singer M S, Kershaw D B, Rosen S D. Identification of podocalyxin-like protein as a high endothelial venule ligand for L-selectin: parallels to CD34. J. Exp. Med. 1998;187:1965-1975.
Streeter P R, Rouse B T N, Butcher E C. Immunohistologic and functional characterization of a vascular addressin involved in lymphocyte homing into peripheral lymph nodes. J. Cell Bio. 1988; 107:1853-1862.
Baumhueter S, Singer M S, Henzel W, et al. Binding of L-selectin to the vascular sialomucin CD34. Science. 1993;262:436-438.
Civin C I, Strauss L C, Brovall C, Fackler M J, Schwartz J F, Shaper J H. Antigenic analysis of hematopoiesis III: A hematopoietic progenitor cell surface antigen defined by a monoclonal antibody raised against KG-1a cells. J. Immunol 1984;133:157-165.
Koeffler H P, Billing R, Lusis A J, Sparkes R, Golde D W. An undifferentiated variant derived from the human acute myelogenous leukemia cell line (KG-1). Blood. 1980;56:265-273.
Francis K, Ramakrishna R, Holloway W, Pallson B O. Two new pseudopod morphologies displayed by the human hematopoietic KG1a progenitor cell line and by primary human CD34+ cells. Blood. 1998;92:3616-3623.
Stamper H B, Woodruff J J. Lymphocyte homing into lymph nodes: in vitro demonstration of the selective affinity of recirculating lymphocytes for high-endothelial venules. J. Exp. Med. 1976;144:828-833.
Oxley S M, Sackstein R. Detection of an L-selectin ligand on a hematopoietic progenitor cell line. Blood. 1994;84:3299-3306.
Sackstein R, Fu L, Allen K L. A hematopoietic cell L-selectin ligand exhibits sulfate-independent binding activity. Blood. 1997;89:2773-2781.
Sako D, Chang X J, Barone K M, et al. Expression cloning of a functional glycoprotein ligand for P-selectin. Cell. 1993;75:1179-1186.
Asa D, Raycroft L, Ma L, et al. The P-selectin glycoprotein ligand functions as a common human leukocyte ligand for P- and E-selectins. J. Biol. Chem. 1995;270:11662-11670.
Walcheck B, Moore K L, McEver R P, Kishimoto T K. Neutrophil-neutrophil interactions under hydrodynamic shear stress involve L-selectin and PSGL-1. J. Clin. Invest. 1996;98:1081-1087.
Guyer D A, Moore K L, Lynam E B, et al. P-selectin glycoprotein ligand-1 (PSGL-1) is a ligand for L-selectin in neutrophil aggregation. Blood. 1996;88:2415-2421.
Spertini O, Cordey A-S, Monai N, Giuffré L L, Schapira M. P-selectin glycoprotein ligand 1 is a ligand for L-selectin on neutrophils, monocytes and CD34+ hematopoietic progenitor cells. J. Cell Biol. 1996;135:523-531.
Tu L, Chen A, Delahunty M D, et al. L-selectin binds to P-selectin glycoprotein ligand-1 on leukocytes: Interactions between the lectin, epidermal growth factor and consensus repeat domains of the selectins determine ligand binding specificity. J. Immunol. 1996;157:3995-4004.
Zhou Q, Moore K L, Smith D F, Varki A, McEver R P, Cummings R D. The selectin GMP-140 binds to sialylated, fucosylated lactosaminoglycans on both myeloid and nonmyeloid cells. J. Cell Biol. 1991;115:557-564.
Tracey J B, Rinder H M. Characterization of the P-selectin ligand on human hematopoietic progenitors. Exp. Hematol. 1996;24:1494-1500.
Zannettino A C W, Berndt M C, Butcher C, Butcher E C, Vadas M A, Simmons P J. Primitive human hematopoietic progenitors adhere to P-selectin (CD62P). Blood. 1995;85:3466-3477.
Laszik K, Jansen P J, Cummings R D, Tedder T F, McEver R P, Moore K L. P-selectin glycoprotein ligand-1 is broadly expressed in cells of myeloid, lymphoid and dendritic lineage and in some nonhematopoietic cells. Blood. 1996;88:3010-3012.
Greenberg A W, Kerr W, Hammer D A. Relationship between selectin-mediated rolling of hematopoietic stem and progenitor cells and progression in hematopoietic development. Blood. 2000;95:478486.
Moore K L, Patel K D, Bruehl R E, et al. P-selectin glycoprotein ligand-1 mediates rolling of human neutrophils on P-selectin. J. Cell Biol. 1995;128:661-671.
Vachino G, Chang X-J, Veldman G M, et al. P-selectin glycoprotein ligand-1 is the major counter-receptor for P-selectin on stimulated T cells and is widely distributed in non-functional form on many lymphocytic cells. J. Biol. Chem. 1995;270:21966-21974.
Kumar R, Camphausen R T, Sullivan F X, Cumming D A. Core2 -1,6-N-acetylglucoaminyltransferase enzyme activity is critical for P-selectin glycoprotein ligand-1 binding to P-selectin. Blood. 1996;88:3872-3879.
De Luca M, Dunlop L C, Andrews R K, et al. A novel cobra venom metalloprotease, mocarhagin, cleaves a 10-amino acid peptide from the mature N terminus of P-selectin Glycoprotein ligand receptor, PSGL-1 and abolishes P-selectin binding. J. Biol. Chem. 1995;270:26734-26737.
Lemonnier F, Mescher M, Sherman L, Burakoff S. The induction of cytolytic T lymphocytes with purified plasma membranes. J. Immunol. 1978;120:1114-1120.
Moore K L, Stults N L, Diaz S, et al. Identification of a specific glycoprotein ligand for P-selectin (CD62) on myeloid cells. J. Cell Biol. 1992;1 18:445-456.
Jung T, Dailey M. Rapid modulation of homing receptors (gp90MEL-14) induced by activators of protein kinase C. J. Immunol. 1990;144:3130-3136
Kishimoto T K, Jutila M A, Butcher E C. Identification of a human peripheral lymph node homing receptor: A rapidly down-regulated adhesion molecule. Proc. Natl. Acad. Sci. USA. 1990;87:244-2248.
Drzeniek R. Substrate specificity of neuraminidases. Histochem. 1973;5:271 290.
Paulson J C, Weinstein J, Dorland L, van Halbeek H, Vliegenthart J F. Newcastle disease virus contains a linkage-specific glycoprotein sialidase. Application to the localization of sialic acid residues in N-linked oligosaccharides of alpha 1-acid glycoprotein. J. Biol. Chem. 1982;257:12734-12738.
Kansas G S. Selectins and their ligands: Current concepts and controversies. Blood. 1996;88:3259-3287.
Moore K L. Structure and function of P-selectin glycoprotein ligand-1. Leuk. Lymph. 1998;29:1-15.
Norgard K E, Moore K L, Diaz S, et al. Characterization of a specific ligand for P-selectin on myeloid cells. A minor glycoprotein with sialylated O-linked oligosaccharides. J. Biol. Chem. 1993;268:12764-12774.
Sako D, Cornes K M, Barones K M, Camphausen R T, Cummings R A, Shaw G D. A sulfated peptide segment at the amino terminus of PSGL-1 is critical for P-selectin binding. Cell. 1995;83:323-331.
Li F, Wilkins P P, Crawley S, Weinstein J, Cummings R D, McEver R P. Post-translational modifications of recombinant P-selectin glycoprotein ligand-1 required for binding to P- and E-selectin. J. Biol. Chem. 1996;27:3255-3264.
Bistrup A, Bhakta S, Lee J K, et al. Sulfotransferases of two specificities function in the reconstitution of high endothelial cell ligands for L-selectin. J. Cell Biol. 1999;145:899-910.
Tangemann K, Bistrup A, Hemmerich S, Rosen S. Sulfation of a high endothelial venule-expressed ligand for L-selectin: Effects on tethering and rolling of lymphocytes. J. Exp. Med. 1999;190:935-941.
Snapp K R, Ding H, Atkins H, Warnke R, Luscinskas F W, Kansas G S. A novel P-selectin glycoprotein ligand-1 monoclonal antibody recognizes an epitope within the tyrosine sulfate motif of human PSGL-1 and blocks recognition of both P-and L-selectin. Blood. 1998;91:154-164.
Fuhlbrigge R C, Kieffer D, Armerding D, Kupper T S. Cutaneous lymphocyte antigen is a specialized form of PSGL-1 expressed on skin-homing T-cells. Nature. 1997;389:978-981.
Finger E B, Puri K D, Alon R, Lawrence M B, von Adrian U H, Springer T A. Adhesion through L-selectin requires a threshold hydrodynamic shear. Nature. 1996;379:266-269.
Puri K D, Finger E B, Gaudernack G, Springer T A. Sialomucin CD34 is the major L-selectin ligand in human tonsil high endothelial venules. J. Cell Biol. 1995;131:261-270.
Sassetti C, Tangemann K, Singer M S, Kershaw D B, Rosen S D. Identification of podocalyxin-like protein as a high endothelial venule ligand for L-selectin: Parallels to CD34. J. Exp. Med. 1998;187:1965-1975.
Maley F, Trimble R B, Tarentino A L, Plummer T H. Characterization of glycoproteins and their associated oligosaccharides through the use of endoglycosidases. Anal. Biochem. 1989;180:195-204.
Trimble R B, Tarentino A L. Identification of distinct endoglycosidase (Endo) activities in Flavobacterium meningosepticum: Endo F₁, Endo F₂ and Endo F₃. J. Biol. Chem. 1991;266:1646-1651.
Tarentino A L, Quinone G, Changchien L-M, Plummer T H. Multiple endoglycosidase F activities expressed by Flavobacterium meningosepticum endoglycosidases F₂ and F₃. J. Biol. Chem. 1993;268:9702-9708.
Maly, P, Thall A D, Petryniak B, et al. The α(1,3)fucosyltransferase Fuc-TVII controls leukocyte trafficking through an essential role in L-,E-, and P-selectin ligand biosynthesis. Cell. 1996;86:643-653.
Frenette P S, Mayadas T N, Rayburn H, Hynes R O, Wagner D D. Susceptibility to infection and altered hematopoiesis in mice deficient in both P- and E-selectins. Cell. 1996;184:563-574.
Arbones M L, Ord D C, Ley K, et al. Lymphocyte homing and leukocyte rolling and migration are impaired in L-selectin-deficient mice. Immunity. 1994;1:247 260.

What Is claimed is:

1. An isolated and purified glycoprotein and functional analogues thereof characterized by:
(a) being expressed on at least primitive hematopoietic cells,
(b) being a ligand for L-selectin or E-selectin, the binding of ligand to L-selectin or E-selecting being uninhibited by anti-CD34 antibodies;
(c) being resistant to O-sialoglycoprotein endopeptidase activity;
(d) being unrecognized by MECA-79 a monoclonal antibody which Identifies ligands of L-selectin on lymph node high endothelial venules; and
(e) being sulfation-independent.

2. An isolated and purified glycoprotein and functional analogues thereof as set forth in claim 1 wherein said glycoprotein is a membrane-associated glycoprotein.

3. An isolated and purified glycoprotein and functional analogues thereof as set forth in claim 1 wherein said glycoprotein functions as an adhesion protein ligand.

4. An isolated and purified glycoprotein and functional analogues thereof as set forth in claim 1 wherein said glycoprotein facilitates attachment of lymphocytes to hematopoietic cells.

5. At least one antibody directed against said glycoprotein and functional analogues thereof as set forth in claim 1.

6. A method of targeting cells expressing the glycoprotein as set forth in claim 1 including the steps of:
preparing a monoclonal antibody directed against the glycoprotein as set forth in claim 1,
preparing an immunotoxin utilizing the antibody,
exposing a population of cells to said antibodies, and
killing cells bound to the immunotoxin.

7. The method of claim 6 wherein the toxin is selected from the group consisting of ricin A chain, pseudomonas exotoxin A, diphtheria toxin and chemotherapeutic compounds.

8. The method of claim 6 wherein the cells are exposed to the immunotoxin in vivo.

9. The method of claim 6 further characterized by the cells being selected from the group consisting of leukemic cells, malignant hemopoietic progenitor cells and other malignant cells expressing the glycoprotein.

10. A method of selecting for cells expressing the glycoprotein as set forth in claim 1 including the steps of
preparing an antibody directed against the glycoprotein as set forth in claim 1,
exposing a population of cells to said antibodies, and
selecting cells bound to the antibody.

11. The method of claim 10 wherein said selecting step is selected from complement-mediated lysis; panning, cell sorting.

12. A method of regulating hematopoiesis including the step of.
selecting cells with an appropriate level of expression of the glycoprotein as set forth in claim 1 from a patient
culturing the selected cells, and
reinfusing the patient with the expanded selected cell population.

13. A method of regulating inflammatory response by interrupting cellular migration into lymph nodes and sites of chronic inflammation including the step of administering to a patient functional analogues or antibody directed against the glycoprotein as set forth in claim 1.

14. The method of claim 13 further characterized by the inflammatory response being as found in the group selected from autoimmune disorders, post-ischemic tissue injury and sepsis.

15. A method of performing an overlay adherence assay by using cell suspensions as a substrate.

16. The method of claim 15 further characterized by preparing the single cell suspension substrate by depositing the single cell suspension on a slide using a modified sample chamber for use in a cytocentrifuge.

17. A method of making a cytocentrifuge sample chamber assembly by connecting together a slide and a sample chamber, the sample chamber including a cell substrate depositing port and fixing the cell substrate depositing port at one of a plurality of positions relative to the slide.

18. The method of claim 17 whereby said step of fixing the cell substrate depositing port at one of a plurality of positions relative to the slide is further defined as removing a lateral edge region from an end flange of the sample chamber thereby displacing the sample chamber laterally and/or vertically.

19. A cytocentrifuge sample chamber assembly comprising:
a sample chamber including cell substrate receiving means for receiving a cell substrate and depositing means for depositing a cell substrate on a slide surface during cytocentrifugation; and
connecting means for connecting together a slide and said sample chamber and fixing said depositing means at one of a plurality of positions relative to said connecting means.

20. A cytocentrifuge sample chamber assembly as set forth in claim 19 wherein said connecting means include an end flange with unequal sized side areas to allow lateral displacement of said depositing means.

21. A cytocentrifuge sample chamber assembly as set forth in claim 19 wherein said connecting means include a generally rectangular plate disposed normal to and along an end flange with a removed coextensive lower edge region of both said plate and said end flange to allow vertical displacement of said depositing means and said connecting means include a flat generally rectangular plate disposed normal to and along an end flange with a removed coextensive lower edge region of both said plate and said end flange and unequal sized side areas of said end flange to allow vertical and lateral displacement of said depositing means.

22. A cytocentrifuge sample chamber assembly as set forth in claim 22 wherein said receiving means include a funnel interconnected with said discharge port such that under cytocentrifugation a cell substrate disposed In said funnel enters said discharge port.

23. A kit consisting of a series of incrementally modified sample chambers, said incremental modifications allowing incremental vertical and lateral displacement of a sample chamber in a holder assembly thereby allowing deposition of a call substrate at one of a plurality of positions on a slide.

24. A method of determining a pharmaceutical use by:
modifying L-selectin or E-selectin activity of a cell line; and
applying pharmaceuticals to said cell line which affect the modified selectin activity.

25. The method according to claim 24, wherein said applying step further includes modulating selectin activity with the pharmaceuticals.

26. A cell line expressing the glycoprotein as set forth in claim 1.

27. The cell line according to claim 26, wherein said cell line is developed by positive selection.
